SEQUENCE LISTING (36 sequences)

SEQ ID NO: 1 - DNA, 24 bp, Artificial Sequence
  (Illumina i7 flow cell binding site for immobilisation)
atctcgtatgccgtcttctgcttg

SEQ ID NO: 2 - DNA, 29 bp, Artificial Sequence
  (Illumina i5 flow cell binding site for immobilisation)
aatgatacggcgaccaccgagatctacac

SEQ ID NO: 3 - DNA, 33 bp, Artificial Sequence
  (binding site for at least one oligonucleotide primer - Illumina i7 adapter/primer) (33nt)
gatcggaagagcacacgtctgaactccagtcac

SEQ ID NO: 4 - DNA, 33 bp, Artificial Sequence
  (binding site for at least one oligonucleotide primer - Illumina i5 adapter/primer) (33nt)
acactctttccctacacgacgctcttccgatct

SEQ ID NO: 5 - DNA, 34 bp, Artificial Sequence
  (binding site for at least one oligonucleotide primer - Illumina i7 adapter/primer) (34nt including additional 5′ T)
agatcggaagagcacacgtctgaactccagtcac

SEQ ID NO: 6 - DNA, 33 bp, Artificial Sequence
  (binding site for at least one oligonucleotide primer - Illumina i7 adapter/primer) (33nt) (reverse complement of SEQ ID NO 3)
gtgactggagttcagacgtgtgctcttccgatc

SEQ ID NO: 7 - DNA, 33 bp, Artificial Sequence
  (binding site for at least one oligonucleotide primer - Illumina i5 adapter/primer) (33nt) (reverse complement of SEQ ID NO 4)
agatcggaagagcgtcgtgtagggaaagagtgt

SEQ ID NO: 8 - DNA, 34 bp, Artificial Sequence
  (binding site for at least one oligonucleotide primer - Illumina i7 adapter/primer) (34nt including additional 5′ T) (reverse complement of SEQ ID NO 5)
gtgactggagttcagacgtgtgctcttccgatct

SEQ ID NO: 9 - DNA, 22 bp, Artificial Sequence
  (ONT forward/left end (i5) adapter/primer i.e. binding site for at least one oligonucleotide primer - first adapter)
cttctatctcgctgtccgttca

SEQ ID NO: 10 - DNA, 22 bp, Artificial Sequence
  (ONT reverse/right end (i5) adapter/primer i.e. binding site for at least one oligonucleotide primer - second adapter)
tttctgttggtgctgatattgc

SEQ ID NO: 11 - DNA, 38 bp, Artificial Sequence
  primer
  misc_feature (16)..(16): n is a, c, g, or t; site of barcode (Nx)
atcgcctaccgtgacnacttgcctgtcgctctatcttc

SEQ ID NO: 12 - DNA, 38 bp, Artificial Sequence
  primer
  misc_feature (16)..(16): n is a, c, g, or t; site of barcode (Nx)
atcgcctaccgtgacntttctgttggtgctgatattgc

SEQ ID NO: 13 - DNA, 30 bp, Artificial Sequence
  mutREAD-i5-upper_1
  misc_feature (16)..(18): n is a, c, g, or t; site of Unique Molecular Identifier
cgctcttccgatcthnnnatgagcgatgca

SEQ ID NO: 14 - DNA, 45 bp, Artificial Sequence
  mutREAD-i5-lower_1
  misc_feature (9)..(11): n is a, c, g, or t; site of Unique Molecular Identifier
tcgctcatnnndagatcggaagagcgtcgtgtagggaaagagtgt

SEQ ID NO: 15 - DNA, 30 bp, Artificial Sequence
  mutREAD-i5-upper_2
  misc_feature (16)..(18): n is a, c, g, or t; site of Unique Molecular Identifier
cgctcttccgatcthnnngcctagcgtgca

SEQ ID NO: 16 - DNA, 45 bp, Artificial Sequence
  mutREAD-i5-lower_2
  misc_feature (9)..(11): n is a, c, g, or t; site of Unique Molecular Identifier
cgctaggcnnndagatcggaagagcgtcgtgtagggaaagagtgt

SEQ ID NO: 17 - DNA, 46 bp, Artificial Sequence
  mutREAD-i7-upper_1
  misc_feature (36)..(38): n is a, c, g, or t; site of Unique Molecular Identifier
gtgactggagttcagacgtgtgctcttccgatcthnnncgtgtacc

SEQ ID NO: 18 - DNA, 30 bp, Artificial Sequence
  mutREAD-i7-lower_1
  misc_feature (13)..(15): n is a, c, g, or t; site of Unique Molecular Identifier
aattggtacacgnnndagatcggaagagca

SEQ ID NO: 19 - DNA, 46 bp, Artificial Sequence
  mutREAD-i7-upper_2
  misc_feature (36)..(38): n is a, c, g, or t; site of Unique Molecular Identifier
gtgactggagttcagacgtgtgctcttccgatcthnnngcacatgt

SEQ ID NO: 20 - DNA, 30 bp, Artificial Sequence
  mutREAD-i7-lower_2
  misc_feature (13)..(15): n is a, c, g, or t; site of Unique Molecular Identifier
aattacatgtgcnnndagatcggaagagca

SEQ ID NO: 21 - DNA, 58 bp, Artificial Sequence
  mutREAD-i501
  misc_feature (57)..(58): phosphorothioate bond
aatgatacggcgaccaccgagatctacactatagcctacactctttccctacacgacg

SEQ ID NO: 22 - DNA, 58 bp, Artificial Sequence
  mutREAD-i502
  misc_feature (57)..(58): phosphorothioate bond
aatgatacggcgaccaccgagatctacacatagaggcacactctttccctacacgacg

SEQ ID NO: 23 - DNA, 56 bp, Artificial Sequence
  mutREAD-i701
  misc_feature (57)..(58): phosphorothioate bond
caagcagaagacggcatacgagatcgagtaatgtgactggagttcagacgtgtgct

SEQ ID NO: 24 - DNA, 56 bp, Artificial Sequence
  mutREAD-i702
  misc_feature (55)..(56): phosphorothioate bond
caagcagaagacggcatacgagattctccggagtgactggagttcagacgtgtgct

SEQ ID NO: 25 - DNA, 12 bp, Artificial Sequence
  MutRead-i5-upper_1
gcatgggatgca

SEQ ID NO: 26 - DNA, 45 bp, Artificial Sequence
  MutRead-i5-lower_1
  misc_feature (9)..(12): n is a, c, g, or t; site of Unique Molecular Identifier
  misc_feature (39)..(40): phosphorothioate bond
  misc_feature (40)..(41): phosphorothioate bond
  misc_feature (41)..(42): phosphorothioate bond
  misc_feature (42)..(43): phosphorothioate bond
  misc_feature (43)..(44): phosphorothioate bond
  misc_feature (44)..(45): phosphorothioate bond
tcccatgcnnnnagatcggaagagcgtcgtgtagggaaagagtgt

SEQ ID NO: 27 - DNA, 46 bp, Artificial Sequence
  MutRead-i7-upper_1
  misc_feature (1)..(2): phosphorothioate bond
  misc_feature (2)..(3): phosphorothioate bond
  misc_feature (3)..(4): phosphorothioate bond
  misc_feature (4)..(5): phosphorothioate bond
  misc_feature (5)..(6): phosphorothioate bond
  misc_feature (6)..(7): phosphorothioate bond
  misc_feature (35)..(38): n is a, c, g, or t; site of unique molecular identifier
gtgactggagttcagacgtgtgctcttccgatctnnnngacgtacc

SEQ ID NO: 28 - DNA, 12 bp, Artificial Sequence
  MutRead-i7-lower_1
aattggtacgtc

SEQ ID NO: 29 - DNA, 58 bp, Artificial Sequence; identical to SEQ ID NO: 21
  MutRead-i501
  misc_feature (57)..(58): phosphorothioate bond

SEQ ID NO: 30 - DNA, 58 bp, Artificial Sequence; identical to SEQ ID NO: 22
  MutRead-i502
  misc_feature (57)..(58): phosphorothioate bond

SEQ ID NO: 31 - DNA, 58 bp, Artificial Sequence
  MutRead-i503
aatgatacggcgaccaccgagatctacaccctatcctacactctttccctacacgacg

SEQ ID NO: 32 - DNA, 58 bp, Artificial Sequence
  MutRead-i504
  misc_feature (57)..(58): phosphorothioate bond
aatgatacggcgaccaccgagatctacacggctctgaacactctttccctacacgacg

SEQ ID NO: 33 - DNA, 56 bp, Artificial Sequence; identical to SEQ ID NO: 23
  MutRead-i701
  misc_feature (55)..(56): phosphorothioate bond

SEQ ID NO: 34 - DNA, 56 bp, Artificial Sequence; identical to SEQ ID NO: 24
  MutRead-i702
  misc_feature (55)..(56): phosphorothioate bond

SEQ ID NO: 35 - DNA, 56 bp, Artificial Sequence
  MutRead-i703
  misc_feature (55)..(56): phosphorothioate bond
caagcagaagacggcatacgagataatgagcggtgactggagttcagacgtgtgct

SEQ ID NO: 36 - DNA, 56 bp, Artificial Sequence
  MutRead-i704
  misc_feature (55)..(56): phosphorothioate bond
caagcagaagacggcatacgagatggaatctcgtgactggagttcagacgtgtgct

(19) United States
(12) Patent Application Publication
(10) Pub. No.: US 2024/0093180 A1
(43) Pub. Date: Mar. 21, 2024

(54) OLIGONUCLEOTIDE ADAPTERS AND METHOD

(71) Applicant: Cambridge Enterprise Limited, Cambridge (GB)

(72) Inventors: Rebecca FITZGERALD, Cambridge (GB); Karol NOWICKI-OSUCH, Cambridge (GB); Juliane PERNER, Cambridge (GB)

(21) Appl. No.: 17/999,619

(22) PCT Filed: May 27, 2021
(86) PCT No.: PCT/GB2021/051299

§ 371 Date: Nov. 22, 2022

(30) Foreign Application Priority Data
May 28, 2020 (GB) 2007970.3

(51) Int. Cl. C12N 15/10

(52) U.S. Cl. CPC C12N 15/1065

(57) ABSTRACT

The invention relates to a method of preparing a nucleic acid library from a sample comprising high molecular weight DNA (HMW DNA), preferably genomic DNA, comprising the steps (i) contacting said DNA with a first restriction enzyme and a second restriction enzyme; (ii) contacting said DNA with a pair of oligonucleotide adapters according to any of claims 1 to 12; (iii) contacting said DNA with at least one DNA ligase; and (iv) incubating to allow digestion of the DNA by said first restriction enzyme and second restriction enzyme, annealing of said oligonucleotide adapters to the digested DNA, and ligation of the annealed oligonucleotide adapters to the digested DNA by said at least one DNA ligase. The invention also relates to oligonucleotide adapters, a kit, and uses of same.

### FIELD

The invention is in the field of sequence analysis and in particular library construction from animal genomic DNA, for example mammalian genomic DNA such as human genomic DNA. In particular the invention is in the area of cancer/tumour sequence analysis such as cancer/tumour mutational signature analysis.

### BACKGROUND

Cancer is a genetic disease characterized by enormous mutation burden within the tumour DNA. The mutations accumulate over everyone's lifetime due to exposure to various internal and external DNA damaging agents. This damage will have unique footprints (signatures) left by the specific mutational process, which can be traced by careful analysis of the mutational patterns in DNA. The signatures are mathematically deciphered from the total trinucleotide (mutated base+immediately adjacent bases) substitution counts within the entire genome.

Studies of mutational signatures in tumour DNA increased our understanding of the defective cellular processes implicated in cancer development. This approach can be used for patient stratification and can help tailor therapies targeting specific defects in patient groups. Furthermore, since mutational processes take place before the onset of cancer, studies of mutational signatures have application in cancer prevention programs. Mutational signature studies shed light on the causes of geographic and ethnicity-based differences in cancer incidences. These examples show the significance of mutational signatures.

Despite the importance of signatures in cancer biology, the ability to study them in a clinical setting is limited by current technologies. Gold standard methods such as Whole Genome Sequencing (WGS) require high quality and quantity of DNA extracted from fresh or frozen samples. As the majority of historical samples are stored in formalin-fixed blocks (FFPE), studies of signatures are limited to samples collected specifically for DNA sequencing projects. Despite the recent significant decrease in the cost, WGS procedures are still prohibitively expensive for routine application in the clinical setting.

Genome instability is a hallmark of many cancers and leads to the accumulation of single nucleotide variants and copy number alterations in tumor cells. The analysis of the prevalence of specific nucleotide substitutions throughout the genome has revealed that mutational processes, to which the cells are exposed, leave footprints, termed mutational signatures. Large-scale genome sequencing efforts on different cancer types have identified over 50 mutational signatures and their detailed characterization has improved our understanding of the cellular defects acting on cancer genomes and their evolution in normal tissues. Recent studies have shown that the mutational signatures can be used for patient stratification, for example to help tailor therapies to exploit specific defects in these patient sub-groups or to improve early detection and cancer prevention strategies.

Mutational signatures in a tumor genome usually have been derived from Whole-Genome Sequencing (WGS). Due to the associated sequencing costs, WGS is generally limited to studies with small numbers of high-quality samples, which is a drawback. The successful application of mutational signatures in clinical settings requires availability of a cost-effective, scalable detection method that can handle samples of low quality containing small amounts of DNA and the absence of such a technique is a problem in the art.

The relative contribution of mutational processes to the overall mutational spectrum in DNA samples is deciphered mathematically from the frequency of substitutions in their trinucleotide context. Under the assumption that the frequencies can be accurately estimated from a subset of mutations, sequencing at lower genome-wide coverage, i.e. shallow WGS at 10× coverage (10× sWGS), and whole exome sequencing (WES) have been proposed as potential alternatives to WGS for detecting mutational signatures. However, the low coverage of 10× sWGS can lead to spurious mutations calls and will likely bias the detected mutations to those highly abundant in the cell population of the DNA sample, which is a problem with this approach. On the other hand, WES masks the contribution of intergenic mutations to the mutational spectrum, potentially leading to a biased estimation of the presence of mutational signatures, which is a significant problem.

The prior art can suffer from problems of patient specificity or tumour type specificity (or both).

As noted above, clinical samples are often stored and preserved. Formalin-fixed paraffin-embedded (FFPE) tissue samples are widely used clinically. These are valuable tools in retrospective studies including molecular studies such as DNA analysis. However, it is a problem that degradation of DNA occurs in FFPE samples. This phenomenon has been studied—see for example Guyard et al. 2017 (Virchows Arch. Volume 471 Pages 491-500). This study shows that in samples stored for 5+years, more than 50% of the DNA is lost. Moreover, of what little DNA remains, sequencing performance on that material is drastically reduced—an average 3.3 fold decrease in library yield and an average 4.5 fold increase in the number of single nucleotide variants are found after storage. These are serious barriers to clinically important sequence analysis from FFPE samples, which is a problem in the art.

For sequence analysis, including tumour mutational burden sequence analysis, the ‘gold standard’ in the art is whole genome sequencing (WGS). There are a number of techniques available for this, but all of them are expensive. Faced with this drawback, the alternative approach in the art is to examine the cancer of interest and design a panel of mutations and target only those mutations of interest. This allows a very reduced/abbreviated sequencing effort—only sequencing over very small, short, defined targeted regions containing the particular mutations in a panel of interest. However, this approach is not scalable since the mutation panel has to be separately determined for each tumour type. Moreover, this approach is also not universal because different tumour types or even different patients may have different mutation signatures and if the analysis is confined only to a panel of defined mutations, there is no opportunity to overcome this technical problem using this approach.

Franchini et al disclose the known ‘quaddRAD’ method, a high-multiplexing and PCR duplicate removal double-digest restriction-site-associated DNA (ddRAD) sequencing protocol which produces novel evolutionary insights in a nonradiating cichlid lineage. Franchini et al use a 6 nt barcode. This can lead to problems of instability in annealing of the parts of their adapters together, which is a problem in the art. Franchini et al's adapters are susceptible to nuclease degradation at the termini.

It is a problem in the art that when the sample type is formalin fixed-paraffin embedded (FFPE) material, prior art sequencing techniques such as WGS are ineffective and/or uneconomic.

The present invention seeks to overcome problem(s) associated with the prior art.

### SUMMARY OF THE INVENTION

The inventors drew inspiration from the unconnected field of plant biology.

The inventors have diverged from known techniques in significant manners which are explained in more detail below. In summary, firstly the inventors have made changes to the ligation steps in library production, and have also made changes to adapter design compared to established techniques such as Illumina sequencing. These technical changes are set out in detail below, and lead to technical advantages such as a greater efficiency of ligation, as well as a larger fragment size being retained in the library for analysis. Most importantly, the approaches used herein can increase the incorporation rate of the fragments of interest by approximately 10 times compared to conventional ligation library construction procedures used in (for example) standard Illumina sequencing techniques.

It is important to note that the data quality provided can be “better than” prior art techniques such as WGS—in this sense the sequencing data provided using the invention is of approaching/comparable quality to WGS, but offers the advantage that the invention can be used on problematic sample types such as FFPE. Current WGS approaches if deployed in problematic sample types such as FFPE material are prohibitively expensive. Therefore, a key advance provided by the invention is an extremely cost effective way of obtaining sequence data for problematic sample types such as FFPE material.

The invention may be viewed as an alternative way of studying the genome.

The invention is founded on the idea of combining a known technique from a completely unrelated field (plant biology) in cancer biology. In addition, the invention is also founded on markedly different research protocols/research procedures used in generating sequence information, particularly in library generation before sequence determination is carried out. Thus, the invention is both a new and inventive use of some existing techniques, but crucially is also a new technique/protocol in itself, and also involves new reagents and new materials which have not been used in (for example) library generation in the art.

Thus in one aspect the invention provides a pair of oligonucleotide adapters,
wherein said pair comprises a first oligonucleotide adapter comprising
   (a) a top strand comprising 5′—N₈₋₂₄ barcode sequence—N₁₋₅ sequence corresponding to a sticky end left by digestion by a first restriction enzyme—phosphate—3′
      wherein at least one of the nucleotide(s) of the N₈₋₂₄ barcode sequence immediately adjacent to the N₁₋₅ sequence corresponding to the sticky end left by digestion by said first restriction enzyme is different to the corresponding nucleotide(s) of the recognition sequence of said first restriction enzyme; and
      a bottom strand comprising 5′—phosphate—N₈₋₂₄ barcode sequence complementary to the N₈₋₂₄ barcode sequence of the top strand—N₄₋₂₄ unique molecular identifier (UMI) sequence—binding site for at least one oligonucleotide primer—3′
      wherein at least the 6 bases at the 3′ terminal end of the bottom strand are each phosphorothioated;
and a second oligonucleotide adapter comprising
   (b) a top strand comprising 5′—N₁₋₅ sequence corresponding to sticky end left by a second restriction enzyme—N₈₋₂₄ barcode sequence—3′
      wherein at least one of the nucleotide(s) of the N₈₋₂₄ barcode sequence immediately adjacent to the N₁₋₅ sequence corresponding to the sticky end left by said second restriction enzyme is different to the corresponding nucleotide(s) of the recognition sequence of said second restriction enzyme; and
      a bottom strand comprising 5′—binding site for at least one oligonucleotide primer—N₄₋₂₄ unique molecular identifier (UMI) sequence—N₈₋₂₄ barcode sequence complementary to the N₈₋₂₄ barcode sequence of the top strand—3′
      wherein at least the 6 bases at the 5′ terminal end of the bottom strand are each phosphorothioated;
or
wherein said pair comprises a first oligonucleotide adapter comprising
   (c) a top strand comprising 5′—N₈₋₂₄ barcode sequence—phosphate—3′; and
      a bottom strand comprising 5′—phosphate—N₁₋₅ sequence corresponding to sticky end left by digestion by a first restriction enzyme—N₈₋₂₄ barcode sequence complementary to the N₈₋₂₄ barcode sequence of the top strand—N₄₋₂₄ unique molecular identifier (UMI) sequence—binding site for at least one oligonucleotide primer—3′
wherein at least one of the nucleotide(s) of the N₈₋₂₄ barcode sequence immediately adjacent to the N₁₋₅ sequence corresponding to the sticky end left by digestion by said first restriction enzyme is different to the corresponding nucleotide(s) of the recognition sequence of said first restriction enzyme,
wherein at least the 6 bases at the 3′ terminal end of the bottom strand are each phosphorothioated;
and a second oligonucleotide adapter comprising
   (d) a top strand comprising 5′—N₈₋₂₄ barcode sequence—3′; and
      a bottom strand comprising 5′—binding site for at least one oligonucleotide primer—N₄₋₂₄ unique molecular identifier (UMI) sequence—N₈₋₂₄ barcode sequence complementary to the N₈₋₂₄ barcode sequence of the top strand—N₁₋₅ sequence corresponding to sticky end left by a second restriction enzyme—3′
wherein at least one of the nucleotide(s) of the N₈₋₂₄ barcode sequence immediately adjacent to the N₁₋₅ sequence corresponding to the sticky end left by said second restriction enzyme is different to the corresponding nucleotide(s) of the recognition sequence of said second restriction enzyme,
wherein at least the 6 bases at the 5′ terminal end of the bottom strand are each phosphorothioated.

Without wishing to be bound by theory, in order to aid understanding (a) has a 3′ sticky end/single stranded (ss) overhang e.g. PstI, (b) has a 5′ sticky end/ss overhang e.g. ApoI; (c) has a 5′ sticky end/ss overhang e.g. ApoI on the long strand, and (d) has a 3′ sticky end/ss overhang e.g. PstI on the long strand.

The phosphates as specified above have the advantage of facilitating ligation to the target nucleic acid and/or preventing self-ligation of adapters to one another.

In more detail
suitably the 3′-end of upper adapters is protected with a phosphate group to prevent adapter dimer formation; and/or
suitably the 5′-end of lower adapters contains phosphate group to facilitate ligation of the adapter and target DNA sequence.

In another embodiment the invention relates to a pair of oligonucleotide adapters as described above wherein said oligonucleotide top strand of (a) and/or (c) further comprises a phosphate group at its 5′ terminal end.

This embodiment may also be described by substituting within (a) and (c) above as follows:
(a) a top strand comprising 5′—phosphate—N₈₋₂₄ barcode sequence—N₁₋₅ sequence corresponding to a sticky end left by digestion by a first restriction enzyme—phosphate—3′ . . .
(c) a top strand comprising 5′—phosphate—N₈₋₂₄ barcode sequence—phosphate—3′; . . .

The technical benefit of this embodiment is to enable digestion of the top strand by Lambda Exo exonuclease. This 5′ phosphate facilitates exonuclease digestion and therefore allows and/or improves enzymatic clean-up step(s) e.g. removal of unwanted nucleic acids following ligation.

Suitably said N₈₋₂₄ barcode sequence is a N₈₋₁₂ barcode sequence.

In one embodiment suitably said N₈₋₂₄ barcode sequence is a N₂₄ barcode sequence.

More suitably said N₈₋₂₄ barcode sequence is a N₁₂ barcode sequence.

Most suitably said N₈₋₂₄ barcode sequence is a N₈ barcode sequence.

Suitably the nucleotide of the N₈₋₂₄ barcode sequence immediately adjacent to the N₁₋₅ sequence corresponding to sticky end left by the restriction enzyme is different from the corresponding nucleotide of the recognition sequence of said restriction enzyme. This has the advantage of preventing recutting (redigestion) of the ligated [target DNA-adapter] molecule by the restriction enzyme. This is explained in more detail below.

In one aspect the invention relates to a method of making an oligonucleotide adapter comprising selecting a restriction enzyme leaving a sticky end upon digestion of nucleic acid; noting the nucleotide sequence of the sticky end; specifying a N₈₋₂₄ sample barcode sequence; arranging said N₈₋₂₄ sample barcode sequence adjacent to said nucleotide sequence of the sticky end; comparing said arranged sequence to the recognition sequence of said restriction enzyme and if said arranged sequence comprises said recognition sequence of said restriction enzyme then changing at least one nucleotide of the N₈₋₂₄ sample barcode sequence adjacent to said nucleotide sequence of the sticky end so as to eliminate said recognition sequence of said restriction enzyme and then optionally synthesising an oligonucleotide comprising said changed sample barcode sequence and said nucleotide sequence of the sticky end adjacent to one another. Suitably the nucleotide sequence of the sticky end is at the extreme end of the oligonucleotide.

In another embodiment the invention relates to a pair of oligonucleotide adapters as described above wherein said N₄₋₂₄ unique molecular identifier (UMI) sequence is a N₄₋₁₆ unique molecular identifier (UMI) sequence, preferably a N₄ unique molecular identifier (UMI) sequence.

In another embodiment the invention relates to a pair of oligonucleotide adapters as described above wherein said N₈₋₂₄ barcode sequence is a N₈ barcode sequence, and wherein said N₄₋₂₄ unique molecular identifier (UMI) sequence is a N₄ unique molecular identifier (UMI) sequence.

Suitably the binding site for at least one oligonucleotide primer of the bottom strand of strand of (a) and/or (c) comprises, or consists of, SEQ ID NO: 7, and the binding site for at least one oligonucleotide primer of the bottom strand of strand of (b) and/or (d) comprises, or consists of, SEQ ID NO: 6 or SEQ ID NO: 8.

In another embodiment the invention relates to a pair of oligonucleotide adapters as described above wherein said N₈₋₂₄ barcode sequence is a N₂₄ barcode sequence, and wherein said N₄₋₂₄ unique molecular identifier (UMI) sequence is a N₁₆ unique molecular identifier (UMI) sequence.

Suitably the binding site for at least one oligonucleotide primer of the bottom strand of strand of (a) and/or (c) comprises, or consists of, SEQ ID NO: 9, and the binding site for at least one oligonucleotide primer of the bottom strand of strand of (b) and/or (d) comprises, or consists of, SEQ ID NO: 10.

Suitably said N₄₋₂₄ unique molecular identifier (UMI) sequence is a N₄₋₈ unique molecular identifier (UMI) sequence.

Suitably said N₄₋₂₄ unique molecular identifier (UMI) sequence is a N₄ unique molecular identifier (UMI) sequence.

This has the advantage of minimising adapter size.

Suitably said N₄₋₂₄ unique molecular identifier (UMI) sequence is a N₈ unique molecular identifier (UMI) sequence.

This has the advantage of providing an enhanced number of possible UMI sequences (up to 65536 possible different sequences).

Suitably the top strand N₈₋₂₄ barcode sequence and the bottom strand N₈₋₂₄ barcode sequence complementary to the N₈₋₂₄ barcode sequence of the top strand are present as double stranded nucleic acid within the adapter.

Suitably the N₄₋₂₄ unique molecular identifier (UMI) sequence is present as single stranded nucleic acid within the adapter.

Suitably the N₁₋₅ sequence corresponding to sticky end left by the restriction enzyme is present as single stranded nucleic acid within the adapter.

Suitably the binding site for at least one oligonucleotide primer is present as single stranded nucleic acid within the adapter.

Suitably the binding site for at least one oligonucleotide primer comprises, or consists of, 22 to 34 nucleotides, suitably 22 to 33 nucleotides.

In one embodiment suitably the binding site for at least one oligonucleotide primer comprises, or consists of, 22 nucleotides.

In one embodiment suitably the binding site for at least one oligonucleotide primer comprises, or consists of, 33 nucleotides.

In one embodiment suitably the binding site for at least one oligonucleotide primer comprises, or consists of, 34 nucleotides.

The binding site for at least one oligonucleotide primer may be of a different length in each adapter within a pair of adapters.

In another embodiment the invention relates to a pair of oligonucleotide adapters as described above wherein said first and second restriction enzymes comprise
(i) an enzyme having the recognition site 5′CTGCA/G3′; and
(ii) an enzyme having the recognition site 5′R/AATIY3′

Suitably said first and second restriction enzymes comprise
(i) PstI; and
(ii) ApoI.

In one embodiment suitably the N₁₋₅ sequence of (a) and (c) comprises TGCA and the N₁₋₅ sequence of (b) and (d) comprises AATT.

In one embodiment suitably the N₁₋₅ sequence of (a) and (c) comprises, or consists of, TGCA and the N₁₋₅ sequence of (b) and (d) comprises, or consists of, AATT.

In one embodiment suitably the N₁₋₅ sequence of (a) and (c) comprises, or consists of, AATT and the N₁₋₅ sequence of (b) and (d) comprises, or consists of, TGCA.

By use of PstI/ApoI, this pair of enzymes is in the top 10 enzyme combination for optimal analysis in most tumour types, which is a clear advantage for this embodiment.

In another embodiment the invention relates to a method of preparing a nucleic acid library from a sample comprising high molecular weight DNA (HMW DNA), preferably genomic DNA, comprising the steps
(i) contacting said DNA with a first restriction enzyme and a second restriction enzyme;
(ii) contacting said DNA with a pair of oligonucleotide adapters as described above;
(iii) contacting said DNA with at least one DNA ligase; and
(iv) incubating to allow digestion of the DNA by said first restriction enzyme and second restriction enzyme, annealing of said oligonucleotide adapters to the digested DNA, and ligation of the annealed oligonucleotide adapters to the digested DNA by said at least one DNA ligase.

Suitably said sample comprises fresh frozen tissue.

More suitably said sample comprises formalin fixed paraffin embedded (FFPE) tissue.

In another embodiment the invention relates to a method as described above further comprising:
(iiia) contacting said DNA with any enzyme(s) that reverses the effects of formalin induced DNA degradation and crosslinking.

In another embodiment the invention relates to a method as described above further comprising:
(iiia) contacting said DNA with NEBNext FFPE Repair mix.

In another embodiment the invention relates to a method as described above further comprising:
(v) contacting said DNA with at least one dsDNA specific nuclease and at least one ssDNA specific nuclease and incubating to allow digestion.

In another embodiment the invention relates to a method as described above wherein said dsDNA specific nuclease comprises Lambda exo and said ssDNA specific nuclease comprises ExoI.

Suitably Lambda Exonuclease is as in UniProtKB—P03697. More suitably Lambda Exonuclease is as in NEB Mo262S. Most suitably Lambda Exonuclease means NEB Mo262S.

Suitably Exonuclease I is as in UniProtKB P04995. More suitably Exonuclease I is as in NEB M0293S. Most suitably Exonuclease I means NEB M0293S.

In another embodiment the invention relates to a method as described above further comprising:
(vi) purification of nucleic acid

Suitably purification is by use of an AMPure DNA purification column (Beckman Coulter, Inc., 250 S. Kraemer Blvd., Brea, CA 92821 U.S.A.).

In another embodiment the invention relates to a method as described above further comprising:
(vii) amplification of nucleic acid

Suitably amplification is by PCR (polymerase chain reaction).

In another embodiment the invention relates to a method as described above further comprising:
(viii) selecting nucleic acids in the range 300 to 450 bp.

In another embodiment the invention relates to a method as described above further comprising:
(ix) determining the nucleotide sequence of one or more individual nucleic acid molecule(s)

Suitably amplification and/or sequencing is carried out using a primer comprising
(a) nucleotide sequence complementary to the nucleotide sequence of the binding site for at least one oligonucleotide primer, and
(b) an index barcode sequence, and optionally
(c) a binding site for immobilisation.
wherein said primer has the structure 5′—binding site for immobilisation—index barcode sequence—nucleotide sequence complementary to the nucleotide sequence of the binding site for at least one oligonucleotide primer—3′

Suitably said binding site for immobilisation comprises SEQ ID NO: 1 or SEQ ID NO: 2, or the complement thereof, or the reverse complement thereof.

Suitably said nucleotide sequence complementary to the nucleotide sequence of the binding site for at least one oligonucleotide primer comprises SEQ ID NO: 3 or SEQ ID NO: 4, or the complement thereof, or the reverse complement thereof.

Suitably said index barcode sequence is an N8 index barcode sequence. More suitably said index barcode sequence is an N8 Illumina i7 or i5 sequence as disclosed in Illumina Document #1000000002694 v12; most suitably a sequence selected from the sequences UDI0001 to UDI0096 as disclosed on pages 27-29 of Illumina Document #1000000002694 v12, which is hereby incorporated herein by reference for the nucleotide sequences disclosed.

In another embodiment the invention relates to a method as described above further comprising:
(x) determining a mutational signature from the nucleotide sequence of step (ix)

In another embodiment the invention relates to a method as described above further comprising:
(x) determining a homologous recombination deficiency signature, preferably a HRDetect signature, from the nucleotide sequence of step (ix)

In another embodiment the invention relates to a method as described above further comprising:
(x) identifying a copy number alteration (CNA) from the nucleotide sequence of step (ix)

In another embodiment the invention relates to a kit comprising a pair of oligonucleotide adapters as described above, a DNA ligase and at least two restriction enzymes, each restriction enzyme leaving a different sticky end upon nucleic acid cleavage, and optionally one or more of: buffer, one or more FFPE repair enzyme(s), one or more exonucleases.

In another embodiment the invention relates to use of pair of oligonucleotide adapters as described above or a kit as described above for the generation of a DNA library.

In another embodiment the invention relates to a method for generation of a DNA library, comprising the step of ligation of one or more adapter(s) as described above to one or more double stranded DNA fragment(s) comprising a single stranded overhang at each end of said fragment(s).

In one embodiment the invention related to a method of preparing a nucleic acid library from a sample comprising high molecular weight DNA (HMW DNA), preferably genomic DNA.

Suitably said high molecular weight DNA (HMW DNA), preferably genomic DNA, is derived from formalin fixed paraffin embedded (FFPE) tissue.

In a broad aspect is provided a single adapter as described above i.e. (a) or (b) or (c) or (d); in a broad aspect is provided use of such a single adapter.

### DETAILED DESCRIPTION OF THE INVENTION

In contrast to prior art approaches (see above), the present invention samples random regions of the genome. In this way, the method of the invention is able to produce sequencing information comparable to the quality of WGS approaches. Due to the quasi-random sampling approach (using restriction enzymes—explained in more detail below) a representative sample of the genome is sequenced. This enables the method of the invention to embrace the overwhelming majority of tumour types and therefore is in principle a “universal” approach.

It will be noted that due to the use of restriction enzymes in library preparation, it is not strictly accurate to describe the fragmentation step as “random”. However by carefully choosing the restriction enzymes to be well represented throughout the genome, the fragmentation can indeed approximate to “random” and certainly it is ensured that the fragment generation step is carried out in manner to make it highly representative and so sampling bias and/or user choices of restriction enzyme can be in principle eliminated from the approach.

It will be noted that there are certain tumour types which have a very low mutational burden. For those tumour types, it may be that the method of the invention has a lower efficiency due to the lower incidence of mutations across the whole genome, and therefore a lower absolute number of mutations detected by the sampling of the reduced representation library approach used in the invention. The skilled worker can examine the data from tumours with a lower mutational burden and interpret it accordingly. An example of a tumour type with a low mutational burden is (for example) a brain tumour. In one embodiment, suitably the tumour being analysed using the invention is not a brain tumour.

In one aspect, the invention involves use of an extended inner barcode which allows for a shorter complementary oligo. The shorter complementary oligo has the technical benefit of improving the amount of functionally active oligo's in the samples.

Features/advantages include:
Extended inner barcode sequenced (8 nucleotides instead of 6 used by known quaddRAD);
Extended inner barcode sequences allow for shorter complementary oligo;
Shorter complementary oligo removes theoretical hindrance introduced during annealing of the known complementary oligo. In the known quaddRAD method complementary oligo includes UMI sequence that cannot be accurately matched;
These technical features significantly improve the amount of functionally active oligos in the samples.

Without wishing to be bound by theory, the inventors understand that in the art the UMI has been double-stranded. However, the inventors had the inspiration that the limited ability of double-stranded DNA to find complementary sequences amongst a pool of sequences is a likely hindrance in the reaction. The inventors therefore had the idea to shorten the complementary oligo and improve performance of the reaction. Clearly the resulting nucleic acids remain double-stranded, but in the art the adapter oligo used has been longer and spanned the UMI and therefore the ability to form double-stranded nucleic acids was lower in the prior art approach. A further advantage which is reaped from this innovative approach taken by the inventors is that an enhanced size of the fragments in the libraries is observed. This is a further benefit flowing from the invention.

A further inventive aspect of the approach taken is the use of modified adapters. Currently unligated adapters cannot be removed from reaction mixtures in the art. However, the inventors teach specific phosphorothioated bond type protection in certain adapters such as the first oligonucleotide adapter (sometimes referred to as ‘i5 adapter’ when describing an embodiment of the invention using Illumina sequence determination) and/or the second oligonucleotide adapter (sometimes referred to as ‘i7 adapter’ when describing an embodiment of the invention using Illumina sequence determination) as described herein. By using adapters which have this phosphorothioated protection incorporated, it permits an enzymatic clean-up step to be used. Suitably enzymatic clean-up step means exonuclease digestion step. This enzymatic removal of excess oligo's and/or unligated genomic DNA fragments not only improves the targeting, but also improves the efficiency of the subsequent steps in the process. These technical benefits flow from the teaching provided herein to use the phosphorothioated protection in the first/second oligonucleotide adapters and thereby making them resistant to Lambda Exonuclease digestion which can be used for the clean-up step. It should be noted that this approach is not taken in any library preparations currently known in the art.

Moreover, this step also demonstrates that the inventors protect the nucleic acids from degradation at both ends. Once again, no libraries known in the art achieve this at present.
suitably the 3′-end of lower oligo (bottom strand) of the first oligonucleotide adapter and the 5′-end of lower oligo (bottom strand) of the second oligonucleotide adapter are protected with 6 phosphorothioated bonds;

Lambda Exonuclease is a 5′ exonuclease. Therefore, the nucleic acids protected according to the invention are resistant to degradation by this nuclease.

The choice of length of the oligo's was thoughtfully arrived at by the inventors. The inventors devised a compromise between a “too long” adapter sequence which could be thought of as wasting information since it is necessary to sequence through all of the adapters before reaching the sequence of interest, set against the need to retain the UMI/SB sequences which are important for (for example) multiplexing. In other words, the invention addresses the problem of trading off efficiency of double-stranded DNA formation (which can be raised by using longer oligo's) against the cost of sequencing (which can be reduced by using shorter oligo's). The inventors identified this problem and then devised the solution in the form of the choice of length of oligo's taught herein.

By way of background, prior art approaches use barcodes of 6 to 8 nucleotides, typically 8 nucleotides being the standard length of Illumina sequencing. These are typically placed outside the sequencing/amplification adapters such as Illumina i7/i5 adapters. The 8 nucleotide length is not used as an inner barcode in any prior art approach. ‘Inner barcode’ means located nearest to the target DNA (i.e. the HMW DNA such as genomic DNA) to which the adapter is being annealed/ligated. Thus suitably this refers to an adapter having the general structure 5′—sequencing/amplification adapters such as Illumina i7/i5 adapters—inner barcode—3′, more suitably 5′-sequencing/amplification adapters such as Illumina i7/i5 adapters—UMI—inner barcode—3′ resulting in a ligated [adapter—target DNA—adapter] construct having the general structure:

5′—sequencing/amplification adapter sequence (such as Illumina i7/i5 adapter sequence)—UMI—inner barcode—<target DNA>—inner barcode—UMI—sequencing/amplification adapter sequence (such as Illumina i7/i5 adapter sequence)—3′

Clearly in the final ligated construct there are a number of nucleotides (not shown in the above) between each inner barcode and the corresponding end of the target DNA sequence which nucleotides (not shown in the above) are derived from the restriction enzyme recognition sites used as explained herein.

In a broad aspect, the barcode sequence (sometimes referred to as inner barcode sequence) may be 6-24 nucleotides, more suitably 6-12 nucleotides (i.e. N₆₋₁₂ barcode sequence). More suitably the barcode sequence is 8-24 nucleotides, more suitably 8-12 nucleotides (i.e. N₈₋₁₂ barcode sequence). This provides the advantage of maximised/optimised stability of the double stranded barcode sequence section of the two-stranded adapter sequence.

Most suitably the barcode sequence is not shorter than N8 since barcode sequences shorter than N8 can lead to issues of stability. Most suitably stability is assessed at 37° C. Thus suitably the barcode sequence is at least 8 nucleotides in length, more suitably 8-24 nucleotides in length, more suitably 8-12 nucleotides in length, most suitably 8 nucleotides in length.

Prior art barcode lengths tend to be 6 nucleotides or 8 nucleotides or 12 nucleotides for sample identification. For example, an 8 nucleotide adapter can provide 4\hat{ }8 (i.e. 4 to the power of 8 or 48) combinations thereby enabling multiplex processing of 96 samples at a time. This barcoding is in a different part of the adapter to that which the inventors have varied for improved efficiency of ligation. In other words, the inventors' teachings on oligo composition/length to promote efficient ligation are at a different site on the nucleic acid adapter to the site which is used for barcoding/sample identification. Therefore the choice by the inventors to use an 8 nucleotide oligo for improved stability for enhanced ligation performance (formation of double-stranded DNA) is neither taught nor suggested by the existing use of 8 nucleotide barcodes in other parts of oligo's in prior art approaches.

Regarding protection from Lambda 5′ Exonuclease, it is important to note that the invention is in the context of stranded libraries. This means that the nucleic acid fragments are oriented. This stranding/orientation of the nucleic acid fragments is achieved through the restriction enzyme steps in fragment generation. In this way, it is possible to ensure that the strand of interest is always in the same orientation to (for example) the second adapter (e.g. ‘i7 adapter’). In normal/prior art approaches such as WGS, the strands of interest can be in either orientation. In fact, in prior art/WGS techniques it is a feature of the technique that the strands may be cloned in either orientation since their approach to fragmentation and genome coverage necessitates this. By contrast, the approach described herein to prepare reduced representation libraries has been deliberately designed to be directional/stranded/oriented and therefore differs fundamentally from prior art/known approaches such as WGS. This directionality which is deliberately engineered into the method of the invention is useful to facilitate protection of the directional end from nucleases such as Lambda 5′ Exonuclease.

Similar considerations apply to either of the NGS compatible sequence segments (sometimes called ‘sequencing adapters’) such as the Illumina i5 or the Illumina i7 adapters.

It should be noted that in one aspect the invention relates to a new use of phosphorothioated bond nucleotide protection in adapters for library generation such as directional library generation. It should be emphasised that there is no directional library approach used in the art in connection with WGS. The concept of using a directional library such as a restriction enzyme generated library in cancer biology has never been done before the present invention. The thinking in the art of cancer biology is all about efficiently using WGS techniques to sample the whole genomes. The approach of the present invention represents thinking going against the current view in the art.

Certain directional library approaches have been previously taken in the field of plant biology. However, those plant libraries have suffered from problems of poor efficiency due to the use of adapters different from those produced by the inventors. Moreover, the inventors are not aware of any application of nucleotide protection especially not phosphorothioated bond nucleotide protection being deployed in the production of plant libraries.

The term ‘bases’ refers to nucleotide bases i.e. nucleotide bases within an oligonucleotide unless otherwise apparent from the context.

Suitably the method is a method of preparing a nucleic acid library from a sample comprising mammalian tissue, preferably human tissue.

### Mutational Signatures

Different mutational processes generate unique combinations of mutation types, termed “Mutational Signatures”.

There are many classes of mutation—for example single base substitution, doublet base substitution, small insertions/deletions (‘small’ meaning 1-10 bases/base pairs in this context), as well as larger rearrangements and/or combinations of these mutation types.

Different causes for mutations include environmental carcinogens or UV radiation, or endogenous processes, such as normal mutational decay due to spontaneous deamination of methylated nucleotides, base misincorporation by error-prone polymerases, and unrepaired or incorrectly repaired DNA damage due to impaired DNA damage response (DDR) gene function. Each of these underlying causes leaves a characteristic pattern of mutations, which have been termed ‘mutational signatures’.

Thus, different mutational causes or mutational processes make particular mutation(s) more or less likely. The likelihood of a particular mutation can be dependent on its context in the target polynucleotide e.g. the identity of the neighbouring bases. Therefore a ‘mutational signature’ describes the mutations themselves illuminated by information about the bases immediately 5′ and 3′ to each mutated base, and/or other contextual information e.g. proximity of methylated bases etc.

Mutational signatures are displayed and reported based on the observed trinucleotide frequency of the human genome, i.e., representing the relative proportions of mutations generated by each signature based on the actual trinucleotide frequencies of the reference human genome.

Thus the method of the invention optionally further comprises the step of determining a mutational signature.

In one embodiment determining a mutational signature comprises comparing the sequence information determined for the sample (sample of interest) to reference sequence information from a healthy sample from the same subject (‘reference sample’), and identifying the sequence differences in the sequence information determined for the sample relative to the reference sequence information from said healthy sample from the same subject.

In more detail, suitably the healthy sample from the same subject comprises a sample taken or derived from somewhere else on the subject's body i.e. somewhere other than the sample of interest (sample of interest may be a tumour or cancer sample). Suitably the reference sample comprises, or consists of, a healthy sample from the same subject.

Suitably the reference sample comprises, or consists of, DNA from saliva, or DNA derived from healthy tissue next to tumour. Most suitably the reference sample comprises, or consists of, DNA derived from blood. Our method, since it produces reproducible regions and not random genomic regions, is particularly well suited for somatic mutation calling because you need to scan the same sequence in blood and tumour for somatic mutation calling

This identifies mutations in the sample (sample of interest) relative to the reference sequence information from said healthy sample from the same subject. This is termed ‘mutation calling’. Mutation calling may be done using widely available software such as Mutect2 or Strelka.

Thus in one embodiment calling the mutations comprises using GATK Mutect2 software available from the Broad Institute (e.g. available via GitHub online or from Broad Institute, 415 Main Street, Cambridge, MA 02142, USA).

Thus in one embodiment calling the mutations comprises using Strelka software (e.g. ‘Strelka2 germline and somatic small variant caller’ available via GitHub online or as described in Saunders et al 2012 Bioinformatics vol 28 pages 1811-7).

The determination of a mutational signature may be carried out by examining the sequence context for each of the mutations identified in the above described sequence information (i.e. the ‘calling of mutations’ step). This determination of a mutational signature from the mutations identified from the sequence information generated using the method of the invention is easily accomplished by the person skilled in the art, for example using widely available tools such as the ‘SomaticSignatures R’ package (Gehring JS, Fischer B, Lawrence M, Huber W (2015). “SomaticSignatures: Inferring Mutational Signatures from Single Nucleotide Variants.” Bioinformatics. doi: 10.1093/bioinformatics/btv408).

If required, ‘picard tools’ from the Broad Institute (e.g. available via GitHub online or from Broad Institute, 415 Main Street, Cambridge, MA 02142, USA) may be used for manipulating high-throughput sequencing (HTS) data and formats such as SAM/BAM/CRAM and VCF.

### Number of Mutations

In principle any number of mutations may be used for calling mutational signatures. In principle you can use any number of mutations to derive signature contributions, but the more mutations provided the better the estimate compares to the true mutational signature profile. Estimates for the proportions get better the more examples (mutations) are drawn from the underlying distribution (whatever the mutation processes induce), and hence the mutational signature estimation will be more robust, too. Care should be taken to not bias the process of drawing mutations from the population, which is a problem with known techniques e.g. exome sequencing or shallow whole genome sequencing; this problem is advantageously addressed by the present invention.

Regarding the number of mutations required to call a mutational signature, reference is made to FIG. 1A of P10681GB.

Suitably at least 10 mutations are used for calling signatures, more suitably at least 100 mutations are used, most suitably at least 250 mutations are used, or even more. For example, in tumours that have a diverse set of signatures, more than 250 mutations might be advantageous. In this context the number of mutations, means the number of mutations called for individual sample(s), (rather than the number of mutations present in the entire tumour).

### Restriction Enzymes

The term “restriction enzyme” has its normal meaning in the art i.e. a site specific DNA endonuclease. These enzymes cleave DNA within, or at a defined distance from, their ‘recognition site’ (i.e. the nucleotide sequence specifically recognised by the enzyme.)

The restriction enzymes used herein may be obtained from any suitable source, or may be produced by expression of a nucleic acid encoding them and purification of the resulting recombinant enzyme.

Most suitably the enzymes are obtained from New England Biolabs Inc. (240 County Road, Ipswich, MA 01938-2732, USA) (‘NEB’).

If desired, alternate restriction enzymes with the same recognition site and/or leaving the same ‘sticky end’ overhangs may be substituted for particular exemplary restriction enzymes mentioned herein. Such restriction enzymes having the same recognition sequence and the same specificity are termed isoschizomers. Examples include SpeI and BcuI, ClaI and Bsu15I etc. Suitably a restriction enzyme isoschizomer may be used. In this embodiment the designation of the enzyme should be understood to specify the recognition site/cut pattern and not to specifically require use of a particular single restriction enzyme.

Occasionally there may be a particular advantage gained by use of a named enzyme (rather than use of an isoschizomer). In this situation, use of the named enzyme is preferred. Such situations are typically apparent from the context.

Suitably the restriction enzymes used have an asymmetric cutting pattern in the longitudinal plane of the nucleic acid polymer. This means that suitably the restriction enzymes used leave a single-stranded overhang or ‘sticky end’ upon cutting. This has the advantage of promoting directional ligation or directional annealing of target segments of cut nucleic acid. Suitably restriction enzymes having a symmetric cutting pattern in the longitudinal plane of the nucleic acid polymer leaving a double-stranded end or ‘blunt end’ are not used.

Suitably the restriction enzymes are symmetric cutting restriction enzymes with respect to the nucleotide sequence i.e. transverse to the longitudinal plane of the nucleic acid polymer. This means cutting each strand of the nucleic acid polymer at the same position relative to the nucleotide sequence of that strand. Thus suitably the restriction enzymes are symmetric cutting restriction enzymes with respect to their nucleotide recognition sequence. This is the most common cutting pattern amongst all Type II restriction enzymes.

Most suitably the restriction enzyme cuts at a position within its recognition sequence.

For example the restriction enzyme PstI cuts as follows:

5′CTGCA/G3′
3′G/ACGTC5′

This is an asymmetric cutting pattern in the longitudinal plane of the nucleic acid polymer, because it leaves sticky ends (i.e. single stranded overhangs) upon cleavage. This is a symmetric cutting pattern with respect to the nucleotide recognition sequence (i.e. the transverse plane of the nucleic acid polymer), because each strand is cut at the same position relative to the nucleotide sequence of that strand—the top strand is cut at 5′CTGCA/G3′ and the bottom strand is cut at the same position relative to the sequence of the bottom strand (i.e. 5′CTGCA/G3′.

PstI cuts at a position within its recognition sequence, because the recognition sequence is CTGCAG and the cut is within this sequence (5′CTGCA/G3′).

Suitably said first and second restriction enzymes are different. Suitably said first and second restriction enzymes have different recognition sites. Suitably said first and second restriction enzymes leave different sticky ends upon digestion. Suitably said first and second restriction enzymes leave sticky ends having different nucleotide sequences upon digestion. Suitably said first and second restriction enzymes leave sticky ends of different lengths upon digestion. Suitably said first and second restriction enzymes leave sticky ends (single stranded overhangs) having different numbers of nucleotides upon digestion. Suitably said first and second restriction enzymes leave sticky ends of different orientations (e.g. 5′ overhang or 3′ overhang) upon digestion. In one embodiment suitably said first restriction enzyme leaves a 3′ overhang upon digestion and said second restriction enzyme leaves a 5′ overhang upon digestion. In one embodiment suitably said first restriction enzyme leaves a 5′ overhang upon digestion and said second restriction enzyme leaves a 3′ overhang upon digestion.

In more detail, suitably said first and second restriction enzymes are different. Suitably said first and second restriction enzymes leave different sticky ends (single stranded nucleic acid segments) upon digestion. As used herein the term ‘sticky end’ means single stranded nucleic acid segment; this is the single stranded nucleic acid segment left by digestion of the nucleic acid by the restriction enzyme. Most suitably said first and second restriction enzymes leave sticky ends (single stranded nucleic acid segments) having different nucleotide sequences upon digestion. If said first and second restriction enzymes leave sticky ends (single stranded nucleic acid segments) having the same nucleotide sequences upon digestion, these are considered different if they are in different 5′ and 3′ arrangements; for example a sticky end of 5′AGTC3′ is different from a sticky end of 3′AGTC5′; the nucleotide sequence is in fact different when written in the same conventional 5′->3′ orientation i.e. 5′AGTC3′ is different from 5′CTGA3′. Most suitably said first and second restriction enzymes leave incompatible sticky ends (single stranded nucleic acid segments) i.e. sticky ends which do not anneal. Suitably said first restriction enzyme leaves a first sticky end upon digestion and said second restriction enzyme leaves a second sticky end upon digestion wherein said first sticky end and said second sticky end are not complementary to one another and/or do not anneal to one another.

Suitably the restriction enzymes used may leave either 3′ overhang (e.g. PstI) or 5′ overhang (e.g. ApoI) depending on operator choice.

In one embodiment suitably enzyme(s) leaving 3′ overhangs may be used.

In one embodiment suitably enzyme(s) leaving 5′ overhangs may be used.

In one embodiment suitably a mixture of enzymes leaving both 3′ and 5′ overhangs may be used.

The choice of enzyme used affects the sticky end overhang created in the target DNA and therefore affects the nucleotide sequence of the N₁₅ part of the adapter oligonucleotides; this sequence is suitably specified by reference to the sticky ends left by the chosen restriction enzyme(s).

Restriction Enzymes and Recognition Sites

| | Preferred Restriction | Other Possible |
|---|---|---|
| Recognition site | Enzyme | Restriction Enzymes |
| 5′CTGCA/G3′ | PstI (e.g. NEB catalogue | BspMAI |
| 3′G/ACGTC5′ | number R0140) | |
| | From Providencia stuartii | |
| | 164 (ATCC 49762) | |
| 5′R/AATTY3′ | ApoI (e.g. NEB catalogue | AcsI, XapI |
| 3′YTTAA/R5′ | number R0556) | |
| | From Arthrobacter | |
| | protophormiae | |
| 5′-AA/CGTT-3′ | AcII (e.g. NEB catalogue | Psp1406I |
| | number R0598) (from | |
| | Acinetobacter | |
| | calcoaceticus M4 II): | |
| 5′-RCATG/Y-3′ | NspI(e.g. NEB catalogue | XcelI, BstNSI |
| 3′-Y/GTACR-5′ | number R0602)(from | |
| | Nostoc species C) | |
| 5′-C/TTAAG-3′ | AflII (e.g. NEB catalogue | BfrI, BspTI, BstAFI, |
| 3′-GAATT/C-5′ | number R0520)(from | MspCI, Vha464I |
| | Anabaena flos-aquae | |
| | (CCAP 1403/13f). | |
| 5′-T/TAA-3′ | MseI (e.g. NEB catalogue | SaqAI, Tru1I, Tru9I |
| 3′-AAT/T-5′ | number R0525)(from | |
| | Micrococcus species (R. | |
| | Morgan). | |

### Restriction Enzyme/Ligation Reactions

Suitably steps (i) (ii) and (iii) are carried out in the same reaction vessel i.e. suitably the restriction enzyme digestion step, the contact with adapter step, and the ligation step are carried out in the same reaction vessel.

Suitably steps (i) (ii) and (iii) are carried out simultaneously i.e. suitably the restriction enzyme digestion step, the contact with adapter step, and the ligation step are carried out simultaneously.

In this context ‘simultaneously’ means that the restriction enzyme, the adapter(s) and the ligase are present in the same reaction mixture at the same time. Clearly if the components are stored separately then there will be a short time between the addition of each component as the operator or the machine adding each component loads/discharges the restriction enzyme/adapter(s)/ligase into the reaction vessel, but the key is that a reaction mixture containing each of these three components at the same time is created. Suitably a reaction mixture comprising both the restriction enzyme and the ligase in an active state is created.

In one embodiment the addition of the restriction enzyme, the adapter(s) and the ligase will be considered to be carried out ‘simultaneously’ if they are all active in the reaction mixture at a point when all three are present in said mixture.

In one embodiment the addition of the restriction enzyme, the adapters and the ligase will be considered to be carried out ‘simultaneously’ if they are all added to the reaction mixture within 2 minutes of one another.

In one embodiment the reaction mixture comprises restriction enzyme(s), adapter(s) and ligase wherein both the restriction enzyme(s) and the ligase are active in the reaction mixture.

Suitably a mixture is formed comprising HMW DNA molecules (such as genomic DNA molecule(s)), adapter molecule(s), active restriction enzyme and active ligase.

Suitably steps (i) (ii) and (iii) are carried out in a single reaction vessel.

### Sample

It is an advantage of the invention that the sample type may be frozen or may be fresh or may be formalin fixed-paraffin embedded (FFPE).

Suitably the sample comprises DNA.

Suitably the sample comprises genomic DNA.

Suitably the sample comprises mammalian DNA.

Suitably the sample comprises human DNA.

Suitably the sample comprises tumour or blood cancer DNA, most suitably tumour DNA.

Suitably the sample comprises high molecular weight DNA (HMW DNA). Suitably the sample consists essentially of high molecular weight DNA (HMW DNA). Suitably the sample consists of high molecular weight DNA (HMW DNA). In this context HMW means DNA comprising polymers greater than 30 000 base pairs (>30 000 bp) in length (>50% of sample). Suitably the HMW DNA comprises DNA, such as undamaged DNA, from fresh or frozen samples.

It is an advantage of the invention that the sample may be degraded i.e. the sample may comprise degraded DNA. In this context, degraded DNA may mean fragmented DNA, and/or shortened DNA molecules (e.g. by exonuclease digestion), and/or DNA known to support poor yields in NGS sequencing (see for example Guyard et al 2017 ibid.).

Most suitably in the context of the invention ‘degraded DNA’ means fragmented DNA. Degraded DNA, such as FFPE treated DNA, is usually in the range of 100-2 000 bp (at least 50% of the sample). An example of a tool that provides an unbiased estimated of DNA degradation is available from Agilent (Agilent 2200 TapeStation System and the Agilent Genomic DNA ScreenTape Assay; Agilent Technologies, Inc., Waldbronn, Germany). It calculates DNA integrity number (DIN). DIN <5 would be considered degraded and <2 severely degraded.

The sample may comprise DNA in the range of 100-2 000 bp.

The sample may comprise DNA with DIN <5.

The sample may comprise DNA with DIN <2.

It is an advantage of the invention that the sample may be small i.e. the sample may comprise only a small quantity of DNA. By ‘small’ is meant Soong DNA or less. Suitably the sample comprises Soong or less DNA. More suitably the sample comprises 100 ng or less DNA.

The sample may be a sample of low cellularity. Cellularity refers to the number and type of cells present. In more detail, cellularity relates to the proportion of epithelial cells of interest (e.g. cancer). There are different ways of estimating cellularity in the art and in principle any such technique is suitable, for example it can be calculated from the sequencing data, or most suitably by microscopic examination of the percentage of the microscopy field. ‘Low’ cellularity means <30% (e.g. <30% of the microscopy field). Known techniques such as WGS is prohibitively expensive for such samples. Most people would use >50% cellularity for WGS. Thus it is an advantage of the invention that the sample may be of low cellularity.

Suitably said sample is from a subject suspected of having esophageal adenocarcinoma (EAC).

Suitably said sample comprises, or is derived from, formalin fixed paraffin embedded (FFPE) material.

### Adapter Features

As used herein the term ‘adapter oligonucleotide’ (sometimes abbreviated to ‘adapter’) means a nucleic acid comprising a top strand and a bottom strand wherein at least part of said top strand and at least part of said bottom strand have nucleotide sequences which are complementary to each other. Suitably said nucleotide sequences which are complementary to each other are present in the adapter as double stranded nucleic acid.

Suitably the nucleic acid is deoxyribonucleic acid (DNA).

### Barcode Sequence (Sometimes Referred to as “Sample Barcode” or Inner Barcode)

The sample barcode may be N₈ to N₂₄, more suitably N₈ to N₁₂, most suitably N₈. The sample barcode is used to provide a unique identifier to identify the sample. Suitably each sample from which target DNA/library is prepared is used with a different sample barcode. This advantageously allows a high degree of multiplexing in sequence information collection. For example, if 8 different samples are used e.g. to prepare 8 different libraries, (for example 1 library for each sample from 8 different patients) then in order to save time and save cost it can be helpful to carry out the sequence determination step by mixing all of these samples into a single sequence determination procedure. By using a separate sample barcode for each sample (i.e. for each patient) then the nucleic acids may be mixed, and a common sequence determination procedure carried out. When the sequence information is analysed, then the “reads” or individual nucleotide sequences determined can be allocated to the correct sample (e.g. correct patient) since they will each share the same unique sample barcode. Suitably a different sample barcode nucleotide sequence is used for each sample. This allows advantageously highly efficient multiplexing and reduces demand on sequence determination apparatus as well as the “per patient/per sample” cost of the analysis i.e. by carrying out sequence determination for different samples in the same multiplexed sequence determination reaction, sequence information can be gathered for numerous different samples in parallel bringing down the cost per sample for any given unit cost of sequence determination procedure. Mixing may be carried out at any stage after ligation of the adapters onto the target DNA. Thus, the samples could be mixed before application, or the amplified nucleic acids could be mixed before sequence determination or whenever is appropriate.

Suitably a mixture of nucleic acids is prepared for sequence determination. Suitably said mixture comprises nucleic acids bearing a sample barcode associated with the sample from which those nucleic acids were generated.

In one aspect suitably the adapter comprises an inner barcode 8 nucleotide sequence. This is sometimes referred to as ‘extended’—in this context ‘extended’ clarifies that the known adapter of Franchini 2017 has a 6 nt inner barcode sequence so the adapter described herein is structurally different from the known adapter for being 2 nt longer (‘extended’). This structural feature provides the benefit of multiplexing whilst improving efficiency of sequencing library preparation. Thus in one embodiment Suitably the barcode sequence (sometimes referred to as “sample barcode” or inner barcode) of the adapter of the invention comprises N₈ (i.e. NNNNNNNN).

More suitably the barcode sequence (sometimes referred to as “sample barcode” or inner barcode) of the adapter of the invention consists of N₈ (i.e. NNNNNNNN).

The extension of the barcode sequence to N8 in the invention (compared to N6 as in prior art such as Franchini et al 2017 ibid.) provides technical advantages. We refer to FIG. 12 (sometimes referred to as FIG. 4/FIG. 4.1/FIG. 4.1 of appendix A). In the known ‘quaddRAD’ method of Franchini et al 2017 (shown in the middle panel of FIG. 4.1), the top strand (top oligo) of each adapter of Franchini et al is much longer than the top strand of the adapter of the invention. In addition the top strand (top oligo) of each adapter of Franchini et al overlaps both the UMI and the Illumina compatible sequences. This prior art arrangement has several drawbacks: the inventors believe that this overlap results in non-perfect matching (in the UMI region) of the oligo strands during the formation of the double stranded adapter and lowers the stability of the known adapters. This also leads to lower efficiency of ligation with the known adapters.

By contrast, the adapter of the invention (sometimes referred to as ‘mutREAD’), the shorter upper oligo (top strand) of the adapter to cover only the “sample barcode” sequence plus the ‘sticky end’ for annealing to the restriction enzyme digested HMW DNA provides advantages.

### Upper Oligo (Top Strand) Length

In one embodiment suitably the upper oligo (top strand) of the adapter comprises only the barcode sequence (sometimes referred to as “sample barcode” or inner barcode) and the ‘sticky end’ for annealing to the restriction enzyme digested HMW DNA. Suitably the upper oligo (top strand) of the adapter consists of the barcode sequence (sometimes referred to as “sample barcode” or inner barcode) and the ‘sticky end’ for annealing to the restriction enzyme digested HMW DNA.

Suitably the upper oligo (top strand) of the adapter is 9-29 nucleotides in length. This is suitably made up of (N₈₋₂₄ barcode sequence)+(N₅ sequence corresponding to sticky end left by restriction enzyme) giving total length of 9-29 nucleotides.

More suitably the upper oligo (top strand) of the adapter is 9-17 nucleotides in length. This is suitably made up of (N₈₋₁₂ barcode sequence)+(N₁₋₅ sequence corresponding to sticky end left by restriction enzyme) giving total length of 9-17 nucleotides.

Most suitably the upper oligo (top strand) of the adapter is 9-13 nucleotides in length. This is suitably made up of (N₈ barcode sequence)+(N₁₋₅ sequence corresponding to sticky end left by restriction enzyme) giving total length of 9-13 nucleotides.

More suitably the upper oligo (top strand) of the adapter is 12 nucleotides in length. This is suitably made up of (N8 barcode sequence)+(N4 sequence corresponding to sticky end left by restriction enzyme PstI (4 nt) or restriction enzyme ApoI (4 nt)) giving total length of 12 nucleotides (nt).

In one embodiment the N₁₋₅ sequence corresponding to sticky end left by restriction enzyme is located on the lower oligo (bottom strand) of the adapter. In this embodiment suitably the upper oligo (top strand) of the adapter is 8-24 nucleotides in length. This is suitably made up of (N₈₋₂₄ barcode sequence) giving total length of 8-24 nucleotides. More suitably the upper oligo (top strand) of the adapter is 8-12 nucleotides in length. This is suitably made up of (N₈₋₁₂ barcode sequence) giving total length of 8-12 nucleotides. Most suitably the upper oligo (top strand) of the adapter is 8 nucleotides in length. This is suitably made up of (N₈ barcode sequence) giving total length of 8 nucleotides.

In addition to the shortened upper oligo (top strand) of the adapter compared to known adapters, the inventors also teach extension of the barcode sequence such as N8 barcode sequence (sometimes referred to as “sample barcode” or inner barcode) to 8 nt compared to the 6 nt of the known barcode in Franchini et al 2017.

This provides an improvement in stability. Of course there are two extra nucleotides of sequence information which are ‘sacrificed’ during sequencing by this two nucleotide extension of the barcode sequence such as N8 barcode sequence (sometimes referred to as “sample barcode” or inner barcode). However, the invention performs better than the known method DESPITE this sacrifice of sequence information for each sequencing read. Thus the invention performs better even though it goes against conventional thinking in the art by extending the barcode sequence such as N8 barcode sequence (sometimes referred to as “sample barcode” or inner barcode) even though the skilled person would be motivated to keep N6 barcode or even shorten that barcode to gain sequence information. The invention goes against this view in the art and surprisingly out-performs the art too.

Additional advantages of the extended inner barcode and of the shortened top strand of the adapter include increasing the stability of the double-stranded adapter. These features also aid its ability to efficiently ligate to the target sequences.

The N₈₋₂₄ barcode sequence (‘inner barcode sequence’) immediately adjoins the N₁₋₅ sequence corresponding to the sticky end left by the restriction enzyme. It is possible for the nucleotide of the inner barcode sequence which is immediately adjacent to the N₁₋₅ sequence corresponding to sticky end left by the restriction enzyme to match the corresponding nucleotide in the restriction enzyme recognition site. However, more suitably, the nucleotide of the barcode sequence which is immediately adjacent to the N₁₋₅ sequence corresponding to sticky end left by the restriction enzyme is different from the corresponding nucleotide in the restriction enzyme recognition site. This has the advantage of preventing recutting (redigestion) of the ligated [target DNA-adapter]molecule because by specifying this nucleotide of the inner barcode sequence to be different from the corresponding nucleotide in the restriction enzyme recognition site, the ligated molecule no longer contains the restriction enzyme recognition site and so the restriction enzyme will not recognise (and therefore will not cleave) this site in the ligated nucleic acid. This results in a stable nucleic acid which is impervious to the continued action of the active restriction enzyme in the reaction mixture. This technical effect is achieved through the technical feature of having the nucleotide of the barcode sequence which is immediately adjacent to the N₁₋₅ sequence corresponding to sticky end left by the restriction enzyme be different from the corresponding nucleotide in the restriction enzyme recognition site.

Clearly if the sticky end is shorter e.g. 2 nucleotides then there will be a plurality of nucleotides at the extreme end of the inner barcode sequence adjacent to the N₁₋₅ sequence corresponding to sticky end left by the restriction enzyme which would e ach be in positions corresponding to the recognition site of the restriction enzyme—in this embodiment at least one of the nucleotides present on the inner barcode in a position corresponding to the recognition site of the restriction enzyme is different to the corresponding nucleotide(s) in the restriction enzyme recognition site. These nucleotides are always contiguous and always at a position in the inner barcode immediately adjacent to the N₁₋₅ sequence corresponding to sticky end left by the restriction enzyme.

### EXAMPLE: 4 NT STICKY END (E.G. PSTI, APOI)

### N1SE1SE2SE3SE4

Here N1 represents the final nucleotide of the barcode sequence which is immediately adjacent to the N₁₋₅ sequence corresponding to sticky end left by the restriction enzyme. In this example the N₁₋₅ sequence corresponding to sticky end left by the restriction enzyme is N₄— this is represented by SE1SE2SE3SE4 (where ‘SE’ is a nucleotide). Here N1 is chosen to be different to the corresponding nucleotide in the restriction enzyme recognition site.

### Similarly for a 2 nt Sticky End:

### N1N2SE1SE2

Here N1N2 represent the final 2 nucleotides of the barcode sequence which are immediately adjacent to the N₁₋₅ sequence corresponding to sticky end left by the restriction enzyme. In this example the N₁₋₅ sequence corresponding to sticky end left by the restriction enzyme is N₂— this is represented by SE1SE2 (where ‘SE’ is a nucleotide).

In one embodiment at least one of N1 or N2 is chosen to be different to the corresponding nucleotide in the restriction enzyme recognition site.

In one embodiment more suitably the final nucleotide of the inner barcode (i.e. that nucleotide which is immediately adjacent to the N₁₋₅ sequence corresponding to sticky end left by the restriction enzyme)—in this example N2—is chosen to be different from the corresponding nucleotide in the restriction enzyme recognition site.

In one embodiment both N1 and N2 are chosen to be different to the corresponding nucleotide in the restriction enzyme recognition site.

Other permutations will be apparent to the skilled reader from the above explanations. Typically the number of nucleotides in the inner barcode which could be chosen to be different from the corresponding nucleotide in the restriction enzyme recognition site for all symmetric cutting restriction enzymes (symmetric cutting in the transverse plane of the nucleic acid polymer i.e. cutting each strand at the same point in the nucleotide sequence of that strand) may be determined by:

((number of nucleotides in restriction enzyme recognition site)−(number of nucleotides in sticky end generated from restriction enzyme cleavage))/2

So for PstI:

recognition site with cut site marked: 5′-CTGCA/G-3′

((6 nucleotides in restriction enzyme recognition site)−(4 nucleotides in sticky end generated from restriction enzyme cleavage))/2=ONE nucleotide in the inner barcode could be chosen to be different from the corresponding nucleotide in the restriction enzyme recognition site.

So for AcII (Acinetobacter calcoaceticus M4 II):

recognition site with cut site marked: 5′-AA/CGTT-3′

((6 nucleotides in restriction enzyme recognition site)-(2 nucleotides in sticky end generated from restriction enzyme cleavage))/2=TWO nucleotides in the inner barcode could be chosen to be different from the corresponding nucleotide in the restriction enzyme recognition site. These nucleotides are always contiguous and always at a position in the inner barcode immediately adjacent to the N₁₋₅ sequence corresponding to sticky end left by the restriction enzyme.

Suitably at least one of the nucleotide(s) of the N₈₋₂₄ barcode sequence immediately adjacent to the N₁₋₅ sequence corresponding to the sticky end left by the restriction enzyme of (i) is different to the corresponding nucleotide(s) of the recognition sequence of the restriction enzyme of (i).

More suitably each of the nucleotide(s) of the N₈₋₂₄ barcode sequence immediately adjacent to the N₁₋₅ sequence corresponding to the sticky end left by the restriction enzyme of (i) is different to the corresponding nucleotide(s) of the recognition sequence of the restriction enzyme of (i).

Suitably the inner barcode does not comprise a recognition sequence for the restriction enzymes used. Suitably a population of inner barcode sequences is used i.e. a population of adapter molecules in which the inner barcode is prepared to be randomised (except for any defined nucleotide(s) e.g. adjacent to the N₁₋₅ sticky end sequence as explained below). Clearly it is possible that within this population of sequences a certain proportion may by chance comprise a restriction enzyme recognition site. If so, these will be cleaved by the restriction enzyme and will be removed by optional purification and/or optional size-selection and/or will fail to amplify at that stage so will not adversely affect the library created.

### Lower Oligo (Bottom Strand) Length

Unless otherwise apparent from the context, ‘lower strand’ or ‘bottom strand’ of the adapter means the longer of the two strands.

Unless otherwise apparent from the context, ‘lower strand’ or ‘bottom strand’ of the adapter means the strand which comprises the binding site for at least one oligonucleotide primer.

Suitably the lower oligo (bottom strand) is up to 200 nucleotides in length. For synthesis of long oligos, 200 nt is a convenient upper limit for efficient DNA synthesis.

More importantly, the ability of the single stranded DNA to form secondary structures may be taken into account. Thus suitably the lower oligo (bottom strand) is up to 80 nt in length. The sequence might require some optimisation to maximise stability when the oligo is over >60 nt; thus suitably the lower oligo (bottom strand) is up to 60 nt in length.

In one embodiment suitably the lower oligo (bottom strand) of the adapter comprises only the binding site for at least one oligonucleotide primer, the UMI sequence, and the barcode sequence (sometimes referred to as “sample barcode” or inner barcode). In one embodiment suitably the lower oligo (bottom strand) of the adapter consists of only the binding site for at least one oligonucleotide primer, the UMI sequence, and the barcode sequence (sometimes referred to as “sample barcode” or inner barcode).

Suitably the lower oligo (bottom strand) of the adapter is 45-82 nucleotides in length. This is suitably made up of (binding site for at least one oligonucleotide primer)+(N₈₋₂₄ barcode sequence)+(N₄₋₂₄ UMI sequence) giving total length of 45-81 nucleotides for a (e.g.) 33 nt binding site for at least one oligonucleotide primer, or 46-82 nucleotides for a (e.g.) 34 nt binding site for at least one oligonucleotide primer.

More suitably the lower oligo (bottom strand) of the adapter is 45-46 nucleotides in length. This is suitably made up of (binding site for at least one oligonucleotide primer e.g. 33 or 34 nt)+(N₈ barcode sequence)+(N₄ UMI sequence) giving total length of 45 or 46 nucleotides.

More suitably the lower oligo (bottom strand) of the adapter is 73-74 nucleotides in length. This is suitably made up of (binding site for at least one oligonucleotide primer e.g. 33 or 34 nt)+(N₂₄ barcode sequence)+(N₁₆ UMI sequence) giving total length of 73 or 74 nucleotides.

In one embodiment suitably the lower oligo (bottom strand) of the adapter comprises the binding site for at least one oligonucleotide primer, the UMI sequence, the barcode sequence (sometimes referred to as “sample barcode” or inner barcode) and the ‘sticky end’ for annealing to the restriction enzyme digested HMW DNA.

In one embodiment suitably the upper oligo (top strand) of the adapter consists of the binding site for at least one oligonucleotide primer, the UMI sequence, the barcode sequence (sometimes referred to as “sample barcode” or inner barcode) and the ‘sticky end’ for annealing to the restriction enzyme digested HMW DNA.

Suitably the lower oligo (bottom strand) of the adapter is 46-87 nucleotides in length. This is suitably made up of (binding site for at least one oligonucleotide primer)+(N₈₋₂₄ barcode sequence)+(N₄₋₂₄ UMI sequence)+(N₁₋₅ sequence corresponding to sticky end left by restriction enzyme) giving total length of 46-80 nucleotides for a 33 nt binding site for at least one oligonucleotide primer, or 47-87 nucleotides for a 34 nt binding site for at least one oligonucleotide primer.

In one embodiment suitably the lower oligo (bottom strand) of the adapter is 49-50 nucleotides in length. This is suitably made up of (binding site for at least one oligonucleotide primer e.g. 33 or 34 nt)+(N₈ barcode sequence)+(N₄ UMI sequence)+(N₁₋₅ sequence corresponding to sticky end left by restriction enzyme e.g. 4 nt) giving total length of 49 or 50 nucleotides.

In one embodiment suitably the lower oligo (bottom strand) of the adapter is 77-78 nucleotides in length. This is suitably made up of (binding site for at least one oligonucleotide primer e.g. 33 or 34 nt)+(N₂₄ barcode sequence)+(N₁₆ UMI sequence)+(N₁₋₅ sequence corresponding to sticky end left by restriction enzyme e.g. 4 nt) giving total length of 77 or 78 nucleotides.

Clearly other defined lengths are possible using the numeric values for the lengths of the components of the adapter strands provided.

5′ phosphorylation

Unless otherwise apparent from the context, ‘upper oligo’ or ‘top strand’ of the adapter means the shorter of the two strands.

Unless otherwise apparent from the context, ‘upper oligo’ or ‘top strand’ of the adapter means the strand which does NOT comprise the binding site for at least one oligonucleotide primer.

Suitably the top strand (upper Oligo) of the adapter of the invention comprises 5′ phosphate (5′ phosphorylation). This has the advantage of facilitating the activity of Lambda Exonuclease.

Thus suitably the 5′ ends of upper oligo (top strand) of the adapter may contain a phosphate group to facilitate Lambda Exonuclease activity.

More suitably the sample barcode sequence of the top strand (upper Oligo) comprises 5′ phosphate (5′ phosphorylation).

Suitably when the top strand (upper Oligo) has the [N₁₋₅ sequence corresponding to sticky end left by the restriction enzyme] at its 5′ end, this is NOT phosphorylated. This provides the benefit of preventing self-ligation of adapters.

Suitably when the top strand (upper Oligo) has the [N₈₋₂₄ barcode sequence] at its 5′ end, this IS phosphorylated. This provides the benefit of promoting lambda exonuclease digestion.

Thus in one embodiment suitably the 5′ end of N₁₋₅ sequence corresponding to sticky end left by the restriction enzyme is not phosphorylated. This provides the benefit of preventing self-ligation of adapters.

Suitably the 3′ end of N₁₋₅ sequence corresponding to sticky end left by the restriction enzyme is phosphorylated. This provides the benefit of preventing self-ligation of adapters.

Suitably the NGS compatible sequence (i.e. the binding site for at least one oligonucleotide primer (e.g. sequencing adapter such as i5/i7 adapter sequence)) does not comprise a recognition sequence for the restriction enzymes used. In any case typically this part of the eventual ligated nucleic acid molecule will still be single stranded whilst in the presence of the active restriction enzymes and so will not be a substrate for those enzymes since those enzymes act on double stranded nucleic acid and so presence of a restriction enzyme recognition site in the binding site for at least one oligonucleotide primer (e.g. NGS compatible sequence (e.g. sequencing adapter such as i5/i7 adapter)) will not adversely affect the library created. If the binding site for at least one oligonucleotide primer contains a recognition sequence for the restriction enzymes used then suitably the restriction enzymes are removed or inactivated before amplification and/or before the nucleic acid is made fully double stranded.

### UMI—(Unique Molecular Identifier)—Sequence

The UMI suitably comprises a N₄ to N₂₄ sequence, more suitably N₄ to N₁₆ sequence, more suitably a N₄ to N₈ sequence. In one embodiment, suitably the UMI comprises a N₄ sequence. In one embodiment suitably the UMI comprises a N₈ sequence.

Suitably the UMI consists of a fully random set of nucleotides within the UMI. The advantage of this approach is that it creates a large population of individual/different adapters bearing the individual/different UMI sequences. The technical benefit delivered by UMI sequences as described is to permit the discarding of PCR duplicates form the sequence data obtained. The principle is that the length of the UMI is selected for the particular application so as to promote the “tagging” of individual ligated target DNA library nucleic acids (i.e. generated by ligation of the adapters to the restriction enzyme digested nucleic acids as described herein) with a unique code (i.e. the UMI nucleotide sequence). After ligation, the ligated nucleic acids are amplified. After this, sequence determination is carried out. When the sequence information is analysed, if multiple sequence reads are discovered each sharing an identical UMI sequence, this is an indication that those are “PCR duplicates”, and multiple occurrences of that sequence should be discarded from the analysis leaving only a single sequence for each unique UMI. The principle is that if particular library members are amplified at a higher efficiency in the amplification reaction mixture, they might otherwise come to dominate or distort the results in the sequence information extracted. However, by including a UMI in the adapter molecules according to the present invention, then any such PCR duplicate sequence information can be correctly reduced to single occurrences i.e. one “read” or nucleotide sequence per ligated nucleic acid created in the library.

Suitably a population of adapters is used, bearing a population of different UMI sequences.

Suitably the adapter comprises single-stranded DNA in the region of the UMI sequence.

This has the advantage of allowing extension of the length of UMI.

In one embodiment suitably the UMI comprises N4-N24. In one embodiment suitably the UMI consists of N4-N24.

In one embodiment suitably the UMI comprises N4-N16. In one embodiment suitably the UMI consists of N4-N16.

In one embodiment suitably the UMI comprises N4-N8. In one embodiment suitably the UMI consists of N4-N8.

In one embodiment suitably the UMI comprises N4. In one embodiment suitably the UMI consists of N4. N4 is sufficient to produce a significant number of diverse barcodes (4\hat{ }4=256). Thus for applications such as reduced representation sequencing/generation of mutational signatures suitably the UMI comprises N4.

In one embodiment suitably the UMI comprises N5 or more. In one embodiment suitably the UMI consists of N5 or more.

In one embodiment suitably the UMI comprises N6. In one embodiment suitably the UMI consists of N6.

In one embodiment suitably the UMI comprises N7. In one embodiment suitably the UMI consists of N7.

In one embodiment suitably the UMI comprises N8. In one embodiment suitably the UMI consists of N8.

An advantage of longer UMI sequences such as N5-N8, particularly N8 UMIs, is that this enables studies of mobile genetic elements (e.g. transposons). Longer UMIs such as N5-N8, particularly N8 UMIs, enable far higher numbers of unique sequences to tag the nucleic acids of interest (e.g. 8 nt long UMI (sometimes called barcode) would give 4\hat{ }8=65536 possibilities). Since there can be multiple copies of mobile genetic elements spread throughout the HMW DNA such as genomic DNA, and since mobile genetic elements share DNA sequences, this technical effect of longer UMIs provides the benefit of being able to study mobile genetic elements. Conversely, in know methods such as quaddRAD by Franchini et al 2017, the length of the UMI cannot be easily scaled up as it would further negatively affect the stability of the adapter. This problem is overcome by the invention.

Suitably a population of UMI sequences is used i.e. a population of adapter molecules in which the UMI is prepared to be randomised (except for any defined nucleotide(s) if required). Clearly it is possible that within this population of sequences a certain proportion may by chance comprise a restriction enzyme recognition site. If so, these will be cleaved by the restriction enzyme and will be removed by optional purification and/or optional size-selection and/or will fail to amplify at that stage so will not adversely affect the library created. In one embodiment suitably the UMI does not comprise a recognition sequence for the restriction enzymes used.

Notwithstanding the above, it is an advantage of the invention that suitably the UMI sequence of the adapters of the invention is single stranded nucleic acid, such as single stranded DNA. This provides the advantage that they are not recognised by restriction enzymes. This is another advantageous reason for the shorter upper oligo/top strand of the adapters of the invention. Suitably the upper oligo/top strand of the adapters of the invention do not contain UMI sequence. Thus suitably the UMI sequence of the adapters of the invention is present as single stranded nucleic acid.

### Phosphorothioated Bonds

Suitably the 3′-end of lower strand (lower oligo/bottom oligo) of the first oligonucleotide adapter (sometimes called ‘i5 adapter’ when describing embodiments using Illumina sequencing) comprises phosphorothioated bonds, most suitably 6 phosphorothioated bonds.

Suitably the 5′-end of lower strand (lower oligo/bottom oligo) of the second oligonucleotide adapter (sometimes called ‘i7 adapter’ when describing embodiments using Illumina sequencing) comprises phosphorothioated bonds, most suitably 6 phosphorothioated bonds.

The technical feature of the phosphorothioated bonds provides the benefit of protecting the correctly orientated nucleic acids bearing the adapters from nuclease digestion. In one embodiment nuclease digestion means contacting the nucleic acid with Lambda Exonuclease and/or Exonuclease I and incubating to allow digestion. This enables an enzymatic clean-up step to be used in the method of the invention.

In one embodiment nuclease digestion means contacting the nucleic acid with a combination of exonuclease III and exonuclease I and incubating to allow digestion.

In one embodiment this step may comprise contacting the nucleic acid with any combination of single-stranded DNA-specific exonuclease (e.g. Exo I, Exo T, RecJf) and double-stranded DNA-specific exonucleases (e.g. Lambda exo, Exo III). In this embodiment suitably the 3′-end of lower strand (lower oligo/bottom oligo) of the first adapter (‘i5 adapter’) and the 5′-end of lower strand (lower oligo/bottom oligo) of the second adapter (‘i7 adapter’) comprise chemical modification suitable to specifically blocked these enzyme activities. Suitably said chemical modification comprises phosphorothioated bonds.

Phosphorothioated bonds are chiral. One stereoisomer is protected from exonuclease digestion, and one stereoisomer is susceptible to exonuclease digestion. Suitably the phosphorothioated bonds are in the protected stereoisomer form. However typically the phosphorothioated bonds are of mixed orientation. In principle fewer than 6 phosphorothioated bonds could be included in the adapter oligo of the invention. In principle each phosphorothioated bond gives 50% protection since on average 50% of the oligos with that bond will be of the protected stereoisomer and 50% will remain susceptible. Thus in principle each additional phosphorothioated bond in the same single nucleic acid molecule improves protection by a further 50% so in principle 1 phosphorothioated bond gives 50% protection, 2 phosphorothioated bonds gives (50%+(50% of 50%))=75% protection, 3 phosphorothioated bonds gives 87.5% protection and so on.

However in practice including 6 phosphorothioated bonds gives complete protection.

Here complete protection means that loss of susceptible oligos from the mixture occurs only at undetectable levels or at de minimis levels or at levels insignificant for calculation/adjustment of oligo concentrations to use for performance of the methods described herein.

Of course fewer phosphorothioated bonds could be used, accepting partial loss/partial degradation of nucleic acids during enzymatic clean-up (i.e. enzymatic exonuclease digestion) step(s). Thus in one embodiment suitably at least 1 base at the 3′ terminal end or 5′ terminal end of the bottom strand is phosphorothioated; more suitably at least 2 bases at the 3′ terminal end or 5′ terminal end of the bottom strand are phosphorothioated; more suitably at least 3 bases at the 3′ terminal end or 5′ terminal end of the bottom strand are phosphorothioated; more suitably at least 4 bases at the 3′ terminal end or 5′ terminal end of the bottom strand are phosphorothioated; more suitably at least 5 bases at the 3′ terminal end or 5′ terminal end of the bottom strand are phosphorothioated; more suitably at least 6 bases at the 3′ terminal end or 5′ terminal end of the bottom strand are phosphorothioated; more suitably at least 7 or more bases at the 3′ terminal end or 5′ terminal end of the bottom strand are phosphorothioated.

Since in practice including 6 phosphorothioated bonds gives complete protection, most suitably 6 bases at the 3′ terminal end or 5′ terminal end of the bottom strand are phosphorothioated. This provides the advantage of maximising protection whilst streamlining production of the oligos by minimising the phosphorothioated bonds required.

In principle the phosphorothioated bond(s) could be included at any location(s) in the oligo and need not be restricted only to the 3′ terminal end or 5′ terminal end of the oligo. However the exonuclease digestion occurs from the end of the nucleic acid and so including the phosphorothioated bonds at the 3′ terminal end or 5′ terminal end of the oligo ensures that no bases are lost from the oligo due to exonuclease digestion. Including the phosphorothioated bonds ‘inside’ the 3′ terminal end or 5′ terminal end of the oligo (‘inside’ meaning closer to the target DNA i.e. closer to the [N₁₅ sequence corresponding to sticky end left by the restriction enzyme] part of the oligo i.e. leaving unphosphorothioated bonds at the extreme 3′ terminal end or 5′ terminal end of the oligo) is likely to result in loss of those bases with unphosphorothioated bonds due to exonuclease digestion and retention of oligo nucleotide sequence only from the point of inclusion of phosphorothioated bonds. Therefore advantageously the phosphorothioated bond(s) are located at the 3′ terminal end or 5′ terminal end of the oligo.

It should be noted that if an exonuclease having dual single- and double-stranded activity is used, and said activity is blocked by such chemical modification, that also finds application in the invention. For example T5 exonuclease has single- and double-stranded exonuclease activity but currently cannot be blocked by phosphorothioated bonds or other DNA modifications and so is NOT currently suitable for use as an exonuclease in the ‘enzymatic clean-up’ step of the method described herein. However, a variant of T5 exonuclease which is blocked by modification of the nucleic acid such as phosphorothioated bonds would be useful in this ‘enzymatic clean-up’ step.

‘Enzymatic clean-up’ is known for removal of PCR primers (e.g. ExoI as it only degrades ssDNA). Other applications of exonucleases are known. However, the inventors assert that to date no library preparation method that they know of uses dsDNA-specific (Lambda exo) and ssDNA-specific (ExoI) nucleases.

It is possible to use only ExoI to remove unused primers from sequencing library preps/amplification but this will not remove primer dimers, unligated DNA nor DNA with an incorrect orientation of adapters (for example DNA fragments with ApoI sites on both ends or PstI sites on both ends).

To use the ‘enzymatic clean-up’ taught herein for a known standard Illumina library prep that uses Y-shaped adapters, one would have to protect both strands of the DNA of the adapters. In this scenario, if the adapters were ligated in a way that formed primer dimers, they would not be cleaned up (digested).

Thus it is clear that the novel arrangement of chemically protected bonds in the adapters of the invention delivers technical benefits and properties which are not present in known adapters. In particular the adapters of the invention facilitate ‘enzymatic clean-up’ using both ssDNA and dsDNA exonucleases, which is not possible using known adapters as explained above.

### Adapter Oligo Strands

Suitably the adapter top strand and adapter bottom strand are joined via hydrogen bonding between the top strand N₈ barcode sequence and the bottom strand N₈ barcode sequence complementary to the N₈ barcode sequence of the top strand. Suitably said hydrogen bonding is conventional base pairing between the top strand N₈ barcode sequence and the bottom strand N₈ barcode sequence complementary to the N₈ barcode sequence of the top strand. Suitably the top strand N₈ barcode sequence and the bottom strand N₈ barcode sequence are present as double-stranded nucleic acid within the adapter. Suitably the top strand N₈ barcode sequence and the bottom strand N₈ barcode sequence complementary to the N₈ barcode sequence of the top strand are present as double-stranded nucleic acid within the adapter.

In one embodiment suitably the upper oligo (top strand) of the adapter oligonucleotide comprises a single stranded sequence at one end which is complementary to the single stranded overhang created by digestion of the HMW DNA, such as genomic DNA, by the restriction enzyme(s).

In one embodiment suitably the shorter strand (typically the top strand or upper oligo) of the adapter oligonucleotide comprises a single stranded sequence at one end which is complementary to the single stranded overhang created by digestion of the HMW DNA, such as genomic DNA, by the restriction enzyme(s).

The advantage of having sticky ends (i.e. single stranded sequence complementary to that left by the restriction enzyme digestion) on the short oligo is during optimisation of the experiments. It is cheaper to change the restriction enzymes or make other adjustments because only the short oligos would have to be resynthesised when the sticky ends (i.e. single stranded sequence complementary to that left by the restriction enzyme digestion) is present on the short oligo of each adapter.

However it must be noted that it is equally possible to place the sticky ends (i.e. single stranded sequence complementary to that left by the restriction enzyme digestion) on the long oligo (typically the lower strand or bottom strand) of the adapter oligonucleotide. This is easily manufactured by the person skilled in the art following the guidance given herein, but in case any further illustration is required we refer to FIG. 12 (sometimes referred to as “FIG. 1.3”) which shows embodiments with this arrangement.

### Binding Site for at Least One Oligonucleotide Primer

The oligonucleotide primer may be an amplification primer or sequencing primer. The adapter of the invention suitably comprises a nucleotide binding site for one or more oligonucleotide primer(s) such as amplification (e.g. PCR) and/or sequencing primer(s).

Unless otherwise apparent from the context, the binding site for at least one oligonucleotide primer (sometimes referred to as ‘primer binding site’ (sometimes abbreviated to ‘binding site’)) is a region of a nucleic acid molecule having a nucleotide sequence where a primer such as an oligonucleotide primer can bind to start replication. Replication may be for amplification (e.g. PCR) or for sequencing (e.g. NGS). A primer typically comprises single stranded nucleic acid such as RNA or DNA, most suitably DNA.

Primer binding may be referred to as ‘annealing’.

The primer binding site may be on one of the two complementary strands of a double-stranded nucleotide polymer, or may be on a single-stranded nucleotide. The primer typically anneals to the binding site when the binding site is single-stranded, thereby forming a double-stranded nucleic acid across at least the binding site part of the molecule.

Suitably said binding site for at least one oligonucleotide primer (binding site) comprises single-stranded nucleic acid such as single-stranded DNA.

The binding (annealing) means that the nucleotide sequence of the binding site and the complementary nucleotide sequence of the primer undergo base-pairing to form double stranded nucleic acid. Therefore the primer nucleotide sequence and the binding site nucleotide sequence are complementary (i.e. mutually complementary).

Suitably the binding site for at least one oligonucleotide primer (binding site) of said first oligonucleotide adapter is different from the binding site for at least one oligonucleotide primer (binding site) of said second oligonucleotide adapter.

Suitably the nucleotide sequence of the binding site for at least one oligonucleotide primer (binding site) of said first oligonucleotide adapter is different from the nucleotide sequence of said binding site for at least one oligonucleotide primer (binding site) of said second oligonucleotide adapter.

Suitably the binding site for at least one oligonucleotide primer (binding site) of said first oligonucleotide adapter is different in length from said binding site for at least one oligonucleotide primer (binding site) of said second oligonucleotide adapter.

Suitably the binding site for at least one oligonucleotide primer (binding site) of said first oligonucleotide adapter is different in nucleotide sequence from said binding site for at least one oligonucleotide primer (binding site) of said second oligonucleotide adapter.

Suitably the binding site for at least one oligonucleotide primer (binding site) of said first oligonucleotide adapter is different in length and in nucleotide sequence from said binding site for at least one oligonucleotide primer (binding site) of said second oligonucleotide adapter.

Suitably an oligonucleotide primer capable of binding to said at least one oligonucleotide primer (binding site) of said first oligonucleotide adapter is not capable of binding to said at least one oligonucleotide primer (binding site) of said second oligonucleotide adapter.

Suitably the nucleotide sequence of the binding site for at least one oligonucleotide primer (binding site) of said first oligonucleotide adapter and the nucleotide sequence of the binding site for at least one oligonucleotide primer (binding site) of said second oligonucleotide adapter are selected such that an oligonucleotide primer capable of binding to said at least one oligonucleotide primer (binding site) of said first oligonucleotide adapter is not capable of binding to said at least one oligonucleotide primer (binding site) of said second oligonucleotide adapter.

Suitably the nucleotide sequence of the binding site for at least one oligonucleotide primer (binding site) of said first oligonucleotide adapter and the nucleotide sequence of the binding site for at least one oligonucleotide primer (binding site) of said second oligonucleotide adapter are selected such that an oligonucleotide primer capable of binding to said at least one oligonucleotide primer (binding site) of said second oligonucleotide adapter is not capable of binding to said at least one oligonucleotide primer (binding site) of said first oligonucleotide adapter.

Suitably the nucleotide sequence of the binding site for at least one oligonucleotide primer (binding site) of said first oligonucleotide adapter and the nucleotide sequence of the binding site for at least one oligonucleotide primer (binding site) of said second oligonucleotide adapter are selected such that an oligonucleotide primer capable of binding to said at least one oligonucleotide primer (binding site) of said first oligonucleotide adapter is not capable of binding to said at least one oligonucleotide primer (binding site) of said second oligonucleotide adapter, and such that an oligonucleotide primer capable of binding to said at least one oligonucleotide primer (binding site) of said second oligonucleotide adapter is not capable of binding to said at least one oligonucleotide primer (binding site) of said first oligonucleotide adapter.

Suitably said binding site is immediately adjacent to the UMI.

In one embodiment suitably said binding site is 34 nucleotides in length (e.g. i7 compatible binding site).

In one embodiment said binding site is suitably 33 nucleotides in length (e.g. i5 compatible binding site).

In one embodiment suitably said binding site comprises Illumina i5 or i7 compatible sequence.

In one embodiment said binding site comprises ONT compatible sequence.

In one embodiment suitably said binding site comprises i7 compatible sequence selected from SEQ ID NO: 3, SEQ ID NO: 5, SEQ ID NO: 6, SEQ ID NO: 8, or the complement or reverse complement thereof.

In one embodiment suitably said binding site comprises i5 compatible sequence selected from SEQ ID NO: 4, SEQ ID NO: 7, or the complement or reverse complement thereof.

In one embodiment suitably said binding site comprises ONT compatible sequence selected from SEQ ID NO: 9, SEQ ID NO: 10, or the complement or reverse complement thereof.

The amplification/sequencing binding site may be the same site. In other words, primers bearing nucleotide sequence complementary to the amplification/sequencing binding site (binding site for at least one oligonucleotide primer) present in the adapter sequence may be used for amplification and/or sequencing depending on the protocols selected.

### Sequencing Technologies

Numerous sequencing technologies are available in the market.

It will be appreciated that the invention is not in the area of sequencing technology itself—the sequencing technology for nucleotide sequence determination is a matter of operator choice.

Some commentators draw parallels with Moore's Law (referring to the rate of addition of transistors in a microchip doubling yet the cost halving every two years) in the field of sequence determination technology. Therefore it will be clear to the skilled reader that sequencing technology changes quickly and the present invention may need to be implemented with attention to the particular technology it is desired to use for nucleotide sequence determination. In principle any suitable nucleotide sequence determination technique may be used. To aid understanding the invention is illustrated with reference to particularly suitable current nucleotide sequence determination technologies such as those commercially available from Illumina Inc. (ibid.) and/or from Oxford Nanopore Technologies (sometimes referred to as ‘ONT’ or ‘Nanopore’) of Gosling Building, Edmund Halley Road, Oxford Science Park, OX4 4DQ, UK. Alternatively IonTorrent from Thermo Fisher Scientific, 168 Third Avenue, Waltham, MA 02451, USA may be used for nucleotide sequence determination.

### Oxford Nanopore (ONT) Sequencing

FIG. 13 (sometimes referred to as “FIG. 1.4”) illustrates how the invention may be implemented using ONT sequencing technology. Exemplary sequences for Oxford Nanopore (ONT) adapters are provided—see also SEQ ID NO: 9 and SEQ ID NO: 10.

We refer to the publicly available ONT Barcodes documents.

In particular we refer to Oxford Nanopore (ONT) PCR Barcoding Kit (SQK-PBK004) and the PCR-cDNA Barcoding Kit (SQK-PCB109).

The top and bottom strand of this primer carry different flanking sequences:

SEQ ID NO: 11
5′ -ATCGCCTACCGTGAC-barcode-ACTTGCCTGTCGCTCTATCTT
C-3′
SEQ ID NO: 12
5′-ATCGCCTACCGTGAC-barcode-TTTCTGTTGGIGCTGATATTGC-
3′

The top and bottom sequences are different to avoid 5′ and 3′ end sequences annealing to each other and forming a loop.

In particular the binding site for at least one oligonucleotide primer may comprise, or may consist of, the underlined sequence above.

In particular the binding site for at least one oligonucleotide primer may comprise, or may consist of, the bold sequence above.

In one embodiment the binding site for at least one oligonucleotide primer of the bottom strand of (a) and/or (c) may comprise, or may consist of, the underlined sequence above, or the complement or reverse complement thereof, and the binding site for at least one oligonucleotide primer of the bottom strand of (b) and/or (d) may comprise, or may consist of, the bold sequence above, or the complement or reverse complement thereof.

In one embodiment the binding site for at least one oligonucleotide primer of the bottom strand of (a) and/or (c) may comprise, or may consist of, the bold sequence above, or the complement or reverse complement thereof, and the binding site for at least one oligonucleotide primer of the bottom strand of (b) and/or (d) may comprise, or may consist of, the underlined sequence above, or the complement or reverse complement thereof.

FIG. 1.4 highlights how the invention can work with ONT sequencing.

When using ONT sequencing, longer sample barcodes and/or longer UMIs are desirable. Using these longer barcodes and/or longer UMIs does not adversely raise the cost of ONT sequencing.

The table below summarises differences in implementation using exemplary alternate sequence determination technologies.

| Adapter | | |
|---|---|---|
| Feature | Illumina Sequencing | Nanopore Sequencing (ONT) |
| Preferred | N8 to N12 | N8 to N24 |
| Sample | | |
| Barcode | | |
| Preferred | N4 to N16 | N4 to N24 |
| UMI | | |
| Technical | Shorter read e.g. 150 | Higher error rate (96% |
| Benefits/ | nucleotides | accuracy = 4% error rate) |
| Advantages; | Very accurate | Lower/negligible cost per base |
| Differences | Use of shorter | consideration |
| | barcodes | Longer reads obtained |
| | advantageously | Longer sample/UMI barcodes |
| | gains sequence | advantageous—longer |
| | information | barcodes improve accurate |
| | Use of shorter | identification of sequence |
| | barcodes compatible | samples even allowing for |
| | with high read | higher error rate |
| | accuracy | allows for real time sequencing |
| | | (so can be stop at any time) |
| | | with a portable sequencer |
| | | power from any PC |

### Illumina Sequencing

When using Illumina sequencing, sometimes i5/i7 barcodes are used. The i5 or i7 barcode (sometimes called “1₅/i7 bases in adapter” when discussing the Illumina adapters; the complementary sequence may be referred to as “i7 bases for sample sheet”) represents a barcode for multiplexing which is introduced at the amplification/sequence determination step. i5/i7 barcodes are suitably not present on the adapters of the invention. Suitably the i5/i7 barcode is present on a primer used for amplification of the ligated nucleic acids (i.e. nucleic acids comprising an adapter of the invention ligated to the target nucleic acid). The operation of this multiplexing is conventional/known in the art. As will be immediately appreciated, this conventional multiplexing can be operated in addition to/simultaneously with the sample barcode (‘inner barcode’) present on the adapters of the invention as described above. Thus, the adapters according to the invention may be used to provide a second “layer” or opportunity for multiplexing within known opportunities for multiplexing already implemented in the art. Thus, it is an advantage of the invention that the sample barcode described above delivers an even higher level of multiplexing than is currently achieved in the art.

### Further Aspects

Suitably the method further comprises the step: deriving a mutational signature from the nucleotide sequence information from step (ix).

Suitably the method further comprises the step: determining a mutational signature from the nucleotide sequence information obtained.

Suitably the method further comprises the step: inferring from the nucleotide sequence information obtained whether a DNA copy number change is present in the sample.

Suitably said DNA copy number change is a chromosomal duplication.

### Manufacture/Production

The oligonucleotide adapters of the invention can be made or produced according to standard techniques known in the art.

Phosphorothioation of bonds within the oligonucleotide may be done by any technique known in the art.

Phosphorylation of the 5′ and/or 3′ termini of the oligonucleotide may be done by any technique known in the art.

Numerous commercial manufacturer(s) who can produce oligonucleotide(s) such as those described herein are known, and exemplary companies or providers are mentioned in the examples below.

For example oligonucleotide(s) may be obtained from Integrated DNA Technologies, Inc., 1710 Commercial Park, Coralville, Iowa 52241, USA.

### Applications and Advantages

It is an advantage of the invention that it enables assessment of mutational signature(s) in a more straightforward manner compared to prior art whole genome sequencing (WGS). This simplifies the procedure and reduces cost.

It is a key advantage of the invention that the data produced are comparable in quality to WGS data. It is an advantage of the invention that the data is produced for only approximately 10-20% of the price of WGS (at current rates). Therefore, this cost saving alone provides advantages to the invention opening avenues for cancer biology which were previously closed due to excessive cost. This may allow mutation capture which otherwise is prohibitively expensive.

The invention provides the advantage of sequencing to a greater depth of smaller regions than are typically addressed using WGS. This is crucial for samples of low cellularity. It is noted that most clinical samples are samples of low cellularity. In a practical sense, clinical samples are most commonly mixed with normal tissue i.e. the sample available for analysis will contain a proportion of the diseased tissue or cancer tissue mixed together with a proportion of normal tissue which has necessarily also been acquired into the sample as a result of the biopsy or sample collection process. Using prior art approaches such as WGS to provide the necessary deep sequence information to analyse such mixed clinical samples leads to a dramatically escalating cost (which cost escalates on an almost exponential scale according to the depth of data required). However, the present invention by employing reduced representation sequence analysis to sample the genomes of the cells in the clinical samples overcomes this problem.

It is a further advantage of the invention that the data obtained allows copy number changes to be called. For example, it can be possible to examine the data obtained according to the invention and reliably deduce that (for example) the patient has a chromosomal duplication. It must be emphasised that exactly the same protocol is used to obtain the same sequencing data as set out herein, but the data is of a quality which allows copy number changes to be detected and declared i.e. it is an advantage of the invention that more data is obtained than with prior art WGS techniques which do not facilitate DNA copy number analysis. For example, Chin et al 2018 (Experimental and Molecular Pathology 104 (2018) 161-169) describe WGS (shallow WGS) which does allow for copy number identification from fresh and FFPE samples, but only from samples with high cellularity (>30%, e.g. >30-50%, our internal simulations). Shallow WGS of Chin et al does not allow for simultaneous identification of mutational signatures. Known techniques such as 50×WGS allow for identification of both, but at much higher cost than the invention herein.

It is an advantage of the invention that by manipulating the restriction enzymes chosen for the fragmentation step, information can be captured about so-called “jumping genes” i.e. mobile genetic elements such as transposons.

Clearly in rare circumstances a patient may bear a mutation at a particular restriction enzyme site. Although this might make it more difficult to obtain sequence signal from that particular fragment of the genome, it will be noted of course that for each mutation at a restriction enzyme site only a single fragment amongst the extremely large population of fragments undergoing analysis will be affected. Therefore, even if a patient does harbour a genetic mutation altering a restriction enzyme site, the overwhelming majority of the sequence data obtained for that patient will still be harvested in the normal manner, which permits the analysis to be carried out according to the invention.

It is an advantage that the invention samples approximately 10% of the bases in a genome.

It is an advantage that a single step combines restriction enzyme digestion, adapter ligation and correction of FFPE-induced artefacts:

This combination has not been taught or suggested by known protocols;

This simplifies the procedure (fewer steps).

The efficiency of library preparation is improved by the design of the adapters.

We teach a novel procedure for removal of unligated adapters and free DNA using Lambda Exonuclease and Exonuclease I. To the best of the inventors' knowledge, this clean-up procedure has not been previously used in library preparation such as sequencing library preparation. The method benefits from the adapter design described herein. Only correctly oriented and ligated libraries will be protected, all remaining unligated DNA, unligated adapters and DNA with incorrectly ligated adapters is/are removed during enzymatic clean-up (exonuclease digestion).

We teach optional clean-up of nucleic acids (e.g. ligated libraries) using (e.g.) AMPure beads. If desired, this step can be omitted to simplify the protocols.

The libraries are suitably amplified using standard PCR.

To make the method compatible with FFPE samples, the inventors optionally include FFPE repair such as using the NEB FFPE repair kit(s) in the first step of library construction. Suitably NEBNext FFPE Repair mix (NEB M6630S), or a corresponding enzyme mixture, is used. The NEBNext FFPE DNA Repair Mix is a cocktail of enzymes formulated to repair DNA, and specifically optimized and validated for repair of FFPE DNA samples. Alternatively SureSeq™ FFPE DNA Repair Mix (Oxford Gene Technology, Begbroke Science Park, Begbroke Hill, Woodstock Road, Begbroke, Oxfordshire, OX5 1PF, UK), or a corresponding enzyme mixture, may be used.

Suitably optional repair is carried out simultaneously with ligation.

Further improvements include:
Shortening of the complementary oligos on the adapters to match only the inner barcode and the extension of the inner barcode to 8 nt. Unlike known quaddRAD, this allows for more efficient formation of the double-stranded adapter and since the top adapter does not overlap the UMI, it allows for more complex and longer UMIs. The possibility of extending the UMIs is useful for application of the method to Copy number analysis, and/or mobile elements analysis.
3′-end of lower strand (lower oligo/bottom oligo) of the first oligonucleotide adapter (sometimes called ‘i5 adapter’ when describing embodiments using Illumina sequencing) and the 5′-end of lower strand (lower oligo/bottom oligo) of the second oligonucleotide adapter (sometimes called ‘i7 adapter’ when describing embodiments using Illumina sequencing) are protected with 6 phosphorothioated bonds. When combined with “any combination of single-stranded DNA-specific exonuclease (e.g. Exo I, Exo T, RecJf) and double-stranded DNA-specific exonucleases (e. g. Lambda exo, Exo III) whose activities can be specifically blocked by any chemical modification of the adapters (e.g. phosphorothioated bonds)”, the protocol allows for the removal of contaminating DNA (unligated fragments, primer dimers, genomic DNA ligated with the same adapter on both ends). In the known quaddRAD method this DNA constitutes a significant proportion of the sample which limits the ability to amplify DNA and potentially introduces unwanted bias in coverage and mutation calling—these are problems with the known method which are overcome by the invention.

Reduced representation sequencing methods have not previously been used outside of the field of the population genomic data that relies on germline single nucleotide polymorphisms that are present at the high penetrance level (50%) and use high-quality fresh samples. Using the present invention we show that:
we can detect low penetrance somatic mutations;
we can work with FFPE samples that are important clinically and contain the largest repositories of cancer specimens.

Thus in one aspect the invention relates to the use of reduced representation sequencing in mutation calling, especially in tumour and/or cancer mutational signature analysis.

Advantages include:
A novel DNA sequencing method that measures the presence of mutations signatures in all types (fresh frozen and formalin fixed-paraffin embedded—FFPE) of clinical and biological samples
Requires as little as 100 ng of FFPE material
provides a simplified protocol that can be performed within 6 hours with 1-hour hands-on work;
A i-fold decrease in the cost of sequencing when compared with gold standard WGS
Does not require specialised equipment
accurately estimates mutational signatures
works with any type of samples including historical FFPE specimen and fresh samples

The invention is sometimes referred to as Mutational Signature Detection by Restriction Enzyme-Associated DNA Sequencing (mutREAD).

The method allows for the estimation of the relative contribution of mutational processes to the overall mutational spectrum in DNA samples. The method generates DNA libraries with a reduced representation of the genome. Enables unprecedented analysis of the archival clinical samples and/or discovery of the mechanisms behind the cancer-related mutational processes.

The invention identifies a sufficient number of high-quality mutation calls throughout the genome supporting estimation of the mutational exposure. The method estimates the contribution of pre-defined mutational signatures to the full mutational profile.

### Further Embodiments

In one embodiment the invention relates to a method for calling mutational signatures.

In this embodiment the invention may be considered to lie in the use of reduced representation sequence information to call mutational signatures.

Thus there is provided a method comprising:
(i) providing reduced representation sequence information from a sample
(ii) calling at least one mutational signature from the reduced representation sequence information of (i).

More suitably in one embodiment the invention relates to a further new use of a method disclosed herein comprising determining reduced representation sequence information from a sample, and calling at least one mutational signature from said reduced representation sequence information.

Suitably the reduced representation sequence information comprises nucleotide sequence information from genomic DNA from said sample.

Suitably the sample is from a subject suspected of having cancer such as esophageal adenocarcinoma.

This method is surprising because it was not known in the art that reduced representation sequence information could support the calling of a mutational signature. As explained above, prior art efforts have been focused on providing as complete as possible sequence information, for example using WGS or WES or other techniques. Therefore the insight of the inventors in realising that in fact reduced representation sequence information can be used to reliably call mutational signatures is a departure from the known approaches that could not be predicted.

The value and effectiveness of this approach is demonstrated herein, in particular with the computational analyses provided which support this approach.

In more detail, the inventors teach the application of the methods mentioned herein for the detection of mutational signatures. These embodiments represent new applications of methods for generating sequence information to call mutational signatures.

Moreover, use of reduced representation sequence information can also be extended to copy number analysis and/or to study of mobile genetic elements.

Thus the inventors assert that use of reduced representation sequencing for mutation signature discovery (and/or mutational signature calling) is a novel application. This is at least because the ability to recall mutational signatures based on information on only a fraction of known mutations has not been known previously, so is disclosed here for the first time.

The inventors have computationally showed that a small proportion of mutations identified in individual tumours is sufficient to accurately recall mutation signatures.

The inventors have showed that this fraction has to be a random subset of mutations (recall from specific regions such as protein-coding exomes is not sufficient).

The inventors have showed that reduced representation sequence information can provide enough mutations to call the mutational signatures independently of tumour type and mutational signature composition.

The insight that random sampling is sufficient to call mutational signatures has not been known (nor suggested) before and currently known approaches in the mutational signature analysis field are focusing on tumour-type or patient-specific signatures using targeted panels or exome sequencing. The method disclosed herein is the first that can be universally applied for random sampling of mutations.

In one embodiment the invention may be applied to the determination or assay or study of other genomic features. For example the invention may be used to provide information for biomarker models such as HRDetect. HRDetect is a sequence information based predictor for detection of homologous recombination (HR)-deficient tumours. In the art, HRDetect has been whole genome sequencing (WGS)-based. However the inventors believe that HRDetect may be carried out using reduced representation sequence information according to the present invention. Thus in one embodiment the invention provides a method as described above further comprising: (x) determining a homologous recombination deficiency signature from the nucleotide sequence of step (ix). Thus in one embodiment the invention provides a method as described above further comprising: (x) determining a weighted model, such as a HRDetect weighted model, from the nucleotide sequence of step (ix).

The HRDetect model is available from (for example) Wellcome Trust Sanger Institute, Hinxton, Cambridge, UK and/or Guys and St Thomas' NHS Trust, London, UK.

In one embodiment the invention relates to a method of preparing a nucleic acid library from a sample comprising high molecular weight DNA (HMW DNA), preferably genomic DNA, comprising the steps
(i) contacting said DNA with at least one restriction enzyme
(ii) contacting said DNA with at least one adapter oligonucleotide
(iii) contacting said DNA with at least one DNA ligase
(iv) incubating to allow digestion of the DNA by the at least one restriction enzyme, annealing of said at least one adapter oligonucleotide to the digested DNA and ligation of the annealed adapter(s) to the digested DNA by said at least one ligase;
   characterised in that said at least one adapter oligonucleotide is an adapter oligonucleotide as described above.

Suitably the restriction enzyme is a Type II DNA restriction enzyme leaving sticky ends upon digestion of the DNA.

Suitably step (ii) comprises contacting said DNA with at least two adapter oligonucleotides; suitably said at least two adapter oligonucleotides do not anneal and/or do not ligate to each other.

In a preferred embodiment the sequencing is carried out using the Illumina platform, the sample barcode comprises a N₈ sample barcode and the UMI comprises a N₄ UMI and said binding site of a first adapter comprises i7 Illumina sequence and said binding site of said second adapter comprises i5 Illumina compatible sequence. This embodiment has the advantage of a minimised UMI length, a minimised sample barcode length (N₈) and therefore a combined “known” sequencing information overhead of 12 nucleotides and so on a 150 nucleotide Illumina sequencing read, 138 nucleotides of novel sequence information is determined (150 nucleotides minus N8 sample barcode minus N₄ UMI=138 nucleotides).

In one embodiment the invention relates to a nucleic acid molecule comprising:
5′—a first adapter as described above—target nucleic acid segment—a second adapter as described above—3′.

In one embodiment the invention relates to a nucleic acid molecule comprising:
3′—a first adapter as described above—target nucleic acid segment—a second adapter as described above—5′.

In one embodiment the first and second adapters are annealed to the target nucleic acid segment.

In one embodiment the first and second adapters are ligated to the target nucleic acid segment by at least one strand.

In one embodiment the first and second adapters and target nucleic acid segment form a contiguous double stranded nucleic acid molecule.

In one embodiment the invention relates to a library of nucleic acid molecules as described above. In one embodiment the invention relates to a population of nucleic acid molecules as described above. Suitably said population comprises a population of different target nucleic acid segments. Suitably said population of different target nucleic acid segments comprises fragments of HMW nucleic acid such as DNA generated by restriction enzyme cleavage (digestion) of said HMW nucleic acid.

### Paired Adapter Embodiments

In one embodiment is described a pair of oligonucleotide adapters,
wherein said pair comprises a first oligonucleotide adapter comprising
(a) a top strand comprising 5′—N₈₋₂₄ barcode sequence—N₁₋₅ sequence corresponding to a sticky end left by digestion by a first restriction enzyme—phosphate—3′
   wherein at least one of the nucleotide(s) of the N₈₋₂₄ barcode sequence immediately adjacent to the N₁₋₅ sequence corresponding to the sticky end left by digestion by said first restriction enzyme is different to the corresponding nucleotide(s) of the recognition sequence of said first restriction enzyme; and a bottom strand comprising 5′—phosphate—N₈₋₂₄ barcode sequence complementary to the N₈₋₂₄ barcode sequence of the top strand—N₄₋₂₄ unique molecular identifier (UMI) sequence—binding site for at least one oligonucleotide primer—3′
   wherein at least the 6 bases at the 3′ terminal end of the bottom strand are each phosphorothioated;
and a second oligonucleotide adapter comprising
(aa) a top strand comprising 5′—N₈₋₂₄ barcode sequence—N₁₋₅ sequence corresponding to a sticky end left by digestion by a second restriction enzyme—phosphate—3′
   wherein at least one of the nucleotide(s) of the N₈₋₂₄ barcode sequence immediately adjacent to the N₁₋₅ sequence corresponding to the sticky end left by digestion by said second restriction enzyme is different to the corresponding nucleotide(s) of the recognition sequence of said second restriction enzyme; and a bottom strand comprising 5′—phosphate—N₈₋₂₄ barcode sequence complementary to the N₈₋₂₄ barcode sequence of the top strand—N₄₋₂₄ unique molecular identifier (UMI) sequence—binding site for at least one oligonucleotide primer—3′
wherein at least the 6 bases at the 3′ terminal end of the bottom strand are each phosphorothioated,
wherein said N₁₋₅ sequence corresponding to the sticky end left by digestion of (a) is different from said N₁₋₅ sequence corresponding to the sticky end left by digestion of (aa);
wherein said binding site for at least one oligonucleotide primer of (a) is different from said binding site for at least one oligonucleotide primer of (aa).

In one embodiment is described a pair of oligonucleotide adapters,
wherein said pair comprises a first oligonucleotide adapter comprising
(b) a top strand comprising 5′—N₁₋₅ sequence corresponding to sticky end left by a first restriction enzyme—N₈₋₂₄ barcode sequence—3′
   wherein at least one of the nucleotide(s) of the N₈₋₂₄ barcode sequence immediately adjacent to the N₁₋₅ sequence corresponding to the sticky end left by said first restriction enzyme is different to the corresponding nucleotide(s) of the recognition sequence of said first restriction enzyme; and a bottom strand comprising 5′—binding site for at least one oligonucleotide primer—N₄₋₂₄ unique molecular identifier (UMI) sequence—N₈₋₂₄ barcode sequence complementary to the N₈₋₂₄ barcode sequence of the top strand—3′
   wherein at least the 6 bases at the 5′ terminal end of the bottom strand are each phosphorothioated;
and a second oligonucleotide adapter comprising
(bb) a top strand comprising 5′—N₁₋₅ sequence corresponding to sticky end left by a second restriction enzyme—N₈₋₂₄ barcode sequence—3′
   wherein at least one of the nucleotide(s) of the N₈₋₂₄ barcode sequence immediately adjacent to the N₁₋₅ sequence corresponding to the sticky end left by said second restriction enzyme is different to the corresponding nucleotide(s) of the recognition sequence of said second restriction enzyme; and
   a bottom strand comprising 5′—binding site for at least one oligonucleotide primer—N₄₋₂₄ unique molecular identifier (UMI) sequence—N₈₋₂₄ barcode sequence complementary to the N₈₋₂₄ barcode sequence of the top strand—3′ wherein at least the 6 bases at the 5′ terminal end of the bottom strand are each phosphorothioated,
wherein said N₁₋₅ sequence corresponding to the sticky end left by digestion of (b) is different from said N₁₋₅ sequence corresponding to the sticky end left by digestion of (bb);
wherein said binding site for at least one oligonucleotide primer of (b) is different from said binding site for at least one oligonucleotide primer of (bb).

In one embodiment is described a pair of oligonucleotide adapters,
wherein said pair comprises a first oligonucleotide adapter comprising
(c) a top strand comprising 5′—N₈₋₂₄ barcode sequence—phosphate—3′; and
   a bottom strand comprising 5′—phosphate—N₁₋₅ sequence corresponding to sticky end left by digestion by a first restriction enzyme—N₈₋₂₄ barcode sequence complementary to the N₈₋₂₄ barcode sequence of the top strand—N₄₋₂₄ unique molecular identifier (UMI) sequence—binding site for at least one oligonucleotide primer—3′
wherein at least one of the nucleotide(s) of the N₈₋₂₄ barcode sequence immediately adjacent to the N₁₋₅ sequence corresponding to the sticky end left by digestion by said first restriction enzyme is different to the corresponding nucleotide(s) of the recognition sequence of said first restriction enzyme,
wherein at least the 6 bases at the 3′ terminal end of the bottom strand are each phosphorothioated;
and a second oligonucleotide adapter comprising
(cc) a top strand comprising 5′—N₈₋₂₄ barcode sequence—phosphate—3′; and
   a bottom strand comprising 5′—phosphate—N₁₋₅ sequence corresponding to sticky end left by digestion by a second restriction enzyme—N₈₋₂₄ barcode sequence complementary to the N₈₋₂₄ barcode sequence of the top strand—N₄₋₂₄ unique molecular identifier (UMI) sequence—binding site for at least one oligonucleotide primer—3′
wherein at least one of the nucleotide(s) of the N₈₋₂₄ barcode sequence immediately adjacent to the N₁₋₅ sequence corresponding to the sticky end left by digestion by said second restriction enzyme is different to the corresponding nucleotide(s) of the recognition sequence of said second restriction enzyme,
wherein at least the 6 bases at the 3′ terminal end of the bottom strand are each phosphorothioated,
wherein said N₁₋₅ sequence corresponding to the sticky end left by digestion of (c) is different from said N₁₋₅ sequence corresponding to the sticky end left by digestion of (cc);
wherein said binding site for at least one oligonucleotide primer of (c) is different from said binding site for at least one oligonucleotide primer of (cc).

In one embodiment is described a pair of oligonucleotide adapters,
wherein said pair comprises a first oligonucleotide adapter comprising
(d) a top strand comprising 5′—N₈₋₂₄ barcode sequence—3′; and
   a bottom strand comprising 5′—binding site for at least one oligonucleotide primer—N₄₋₂₄ unique molecular identifier (UMI) sequence—N₈₋₂₄ barcode sequence complementary to the N₈₋₂₄ barcode sequence of the top strand—N₁₋₅ sequence corresponding to sticky end left by a first restriction enzyme—3′ wherein at least one of the nucleotide(s) of the N₈₋₂₄ barcode sequence immediately adjacent to the N₁₋₅ sequence corresponding to the sticky end left by said first restriction enzyme is different to the corresponding nucleotide(s) of the recognition sequence of said first restriction enzyme,
wherein at least the 6 bases at the 5′ terminal end of the bottom strand are each phosphorothioated;
and a second oligonucleotide adapter comprising
(dd) a top strand comprising 5′—N₈₋₂₄ barcode sequence—3′; and
   a bottom strand comprising 5′—binding site for at least one oligonucleotide primer—N₄₋₂₄ unique molecular identifier (UMI) sequence—N₈₋₂₄ barcode sequence complementary to the N₈₋₂₄ barcode sequence of the top strand—N₁₋₅ sequence corresponding to sticky end left by a second restriction enzyme—3′
wherein at least one of the nucleotide(s) of the N₈₋₂₄ barcode sequence immediately adjacent to the N₁₋₅ sequence corresponding to the sticky end left by said second restriction enzyme is different to the corresponding nucleotide(s) of the recognition sequence of said second restriction enzyme,
wherein at least the 6 bases at the 5′ terminal end of the bottom strand are each phosphorothioated,
wherein said N₁₋₅ sequence corresponding to the sticky end left by digestion of (d) is different from said N₁₋₅ sequence corresponding to the sticky end left by digestion of (dd);
wherein said binding site for at least one oligonucleotide primer of (d) is different from said binding site for at least one oligonucleotide primer of (dd).

It will be noted by the skilled reader that for these paired adapter embodiments, protection from exonuclease digestion is more challenging. Thus when the method of the invention uses paired adapter embodiments, suitably no exonuclease ‘clean-up’ step is used. This may enable both strands of the target nucleic acid to be captured.

### BRIEF DESCRIPTION OF THE DRAWINGS

FIG. 1 shows graphs and diagrams. Mutational signatures computationally simulated or derived with different sequencing methods and method overview.
(A) Cosine similarity (y-axis) of whole genome sequencing (WGS)-derived mutational signatures for 129 EAC samples and signatures derived from random subsets of mutations with increasing size (x-axis). Boxes show the 25% and 75% quartile with the median indicated by the bold line. Whiskers extend to 1.5 times the interquartile range and samples outside this range are indicated as points. Only samples having sufficient number of mutations (at least the number indicated on the x-axis) contribute to the boxes.
(B) Cosine similarity (y-axis) of WGS-derived mutational signatures for 129 EAC samples and signatures derived from subsets of mutations simulating different sequencing approaches (x-axis). Points show the average cosine similarity and whiskers indicate the standard deviation across all 129 EAC samples. Different enzyme combinations were simulated for RR-seq, each shown as a different point. For 10× sWGS, the average across the 21 simulated samples is given as dashed horizontal line and the standard deviation given as dotted line. RR-Seq—reduced representation sequencing, 10× sWGS—10× shallow whole genome sequencing, WES—whole exome sequencing, expanded WES—whole exome sequencing expanded to untranslated regions and miRNAs.
(C) Schematic overview of the individual steps in mutREAD. Details for each step are given in the Methods section. SB—sample barcode, UMI—unique molecular identifier, RE—restriction enzyme.
D) Comparison of the mutational signature profiles for three EAC samples across different sequencing methods (x-axis). Each bar indicates the contribution of the mutational signature (y-axis) to the overall mutational spectrum. Pairwise cosine similarities to WGS for mutREAD, WES and 10× sWGS are indicated above the bars.

FIG. 2 shows diagrams and a table. The invention (mutREAD) reproducibly detects mutational signatures in FFPE samples
A) Comparison of the mutational signature profiles between WGS, fresh-frozen (FF) and FFPE samples for the same three EAC samples as in FIG. 1. Each bar indicates the contribution of the mutational signature (y-axis) to the overall mutational spectrum. Pairwise cosine similarities to WGS for the two mutREAD libraries are indicated above the bars.
B) Cosine similarity between mutational signatures derived from nine additional FFPE and WGS sample pairs and the number of detected mutations in the FFPE samples used to derive the mutational signatures.
C) Reproducibility of the sequenced regions between the first FFPE-derived technical replicate and the blood sample, the second FFPE-derived technical replicate and the blood sample, and between the two technical replicates (x-axis). The bars indicate the size of the overlapping regions in Mpbs (y-axis) for each comparison. Only regions covered at least 10× contribute to the comparison. The second technical replicate was sequenced to lower coverage and we down-sampled the first technical replicate by 50% to approximately match the sequencing coverage for comparison.

FIG. 3 shows Supplementary FIG. 1 which shows plots. The efficiency of RR-seq-based mutational calling across the PCAWG tumor types.
A) The distribution of cosine similarities between the RR-seq computational simulation-derived (best combination of enzyme per tumor type) and WGS-based mutational signatures. Boxes show the 25% and 75% quartile with the median indicated by the bold line. Whiskers extend to 1.5 times the interquartile range and samples outside this range are indicated as points. For each cancer type the number of samples per group (N) is indicated within the x-axis labels.
B) Scatterplot of the log 10-scaled median number of mutations (x-axis) and the median performance of the RR-seq computational simulation-based mutational signatures measured by cosine similarity to the WGS-based mutational signatures (y-axis) per PCAWG cancer type. Each point represents one cancer type.

Abbreviations: Eso-AdenoCa—Esophageal Adenocarcinoma; AdenoCA—Adenocarcinoma; Lymph-BNHL—B-cell Non-Hodgkin Lymphoma; HCC—Hepatocellular Carcinoma; Head-SCC—Head and Neck Squamous Cell Carcinoma; Panc-AdenoCA—Pancreatic Adenocarcinoma; CNS-Medullo—Medulloblastoma and variants; RCC—Renal Clear Cell adenocarcinoma, papillary type; Myeloid-AML—Acute Myeloid Leukaemia; Bone-Osteosarc—Osteosarcoma; Myeloid-MPN—Myeloproliferative neoplasm; Lymph-CLL—Chronic Lymphocytic Leukaemia; Prost-AdenoCa—Prostate Adenocarcinoma; Bone-Epith—Adamantinoma, Chordoma; Panc-Endocrine—Neuroendocrine carcinoma; CNS-PiloAstro—Pilocytic astrocytoma.

FIG. 4 shows Supplementary FIG. 2 which shows box and whisker plots—Summaries of the genome-wide distribution of loci resulting from the different sequencing approaches
A) Bar plot of the number of genome-wide consecutive 1 Mbps bins that are not covered by at least one expected loci in the computational simulation for each RR-seq with different enzyme combinations and (expanded) WES (x-axis).
B) Summary of the number of expected loci per 1 Mbps bin on logarithmic scale (y-axis) for each RR-seq with different enzyme combinations and (expanded) WES (x-axis). Each box shows the 25% and 75% quartile with the median across all genome-wide consecutive 1 Mpbs bins indicated by the bold line. Whiskers extend to 1.5 times the interquartile range and samples outside this range are indicated as points.

FIG. 5 shows Supplementary FIG. 3 which shows images—Optimization of mutREAD library preparation using FLO1 cell line
A) Bioanalyser traces for the optimization of the single step double digestion and ligation. 500 ng of FLO1 genomic DNA was used for ligation of mutREAD adapters in the presence of indicated enzymes and underwent PCR amplification (20 cycles) using Illumina compatible primers. Samples before (−) and after (+) PCR are shown for each enzyme combination. Dilution indicates dilution of samples for bioanalyzer analysis (for samples that exceeded recommended detection range).
B) Bioanalyser traces for different titration of ratios of AMPure beads and ligated DNA solution (50 ul) to optimize the double size selection of the fragments in the library.
C) Bioanalyser traces prepared under optimised PCR cycles conditions. Note significant decrease in the level of ApoI only fragments when compared to 20 PCR cycles (A).
D) Bioanalyser traces showing improved bands for FFPE samples after treatment with FFPE repair mix and library preparation with optimized protocol.

All samples were run using DNA High Sensitivity Bioanalyzer kit with standard DNA ladder. Green and purple bands indicate lower and upper markers respectively.

FIG. 6 shows Supplementary FIG. 4 which shows graphs—Comparison of the expected and sequenced fragment size distribution.
A) Fragment size (x-axis) distribution of sequencing libraries measured on the Tape-station. Electropherograms of DNA fragments from three samples derived from FFPE (neat), Fresh Frozen (FF, 1:4 dilution) and matching blood samples (1:4 dilution) with the average size of libraries highlighted above the plot. LM—lower marker, UM—upper marker, FU—fluorescent units.
B) Fragment size distribution derived from read-pairs mapped to the human genome. Each plot shows the number of fragments (y-axis) for each length in base pairs (x-axis). The fragment length was calculated as the number of base pairs between the 5′ ends of the read mates (including restriction site parts but not adapters or barcode sequences) and summarized to a histogram using Picard's CollectInsertSizeMetrics function.

FIG. 7 shows Supplementary FIG. 5 which shows graphs—Comparison of the fragment size distributions for technical replicates of FFPE samples and blood Fragment size distribution derived from read-pairs mapped to the human genome. Each plot shows the number of fragments (y-axis) for each length in base pairs (x-axis) for the two technical replicates of FFPE tumor samples and the corresponding blood sample per patient. The fragment length was calculated as the number of base pairs between the 5′ ends of the read mates (including restriction site parts but not adapters or barcode sequences) and summarized to a histogram using Picard's CollectInsertSizeMetrics function.

FIG. 8 shows a diagram of the invention—overview.

FIG. 9 shows graphs, charts and tables of Mutation Signature detection using the invention—the invention is shown as ‘v.2’.
A) Electropherogram of mutREAD libraries constructed using v.1 and v.2 version of the protocol. V.2 version includes modified adapters, enzymatic clean-up and AMPure optional AMPure purification;
B) Table summarising the efficiency of constructed libraries as measure by amount of DNA, mean size of the libraries and enrichment over v.1;
C) Bar chart of the enrichment of library quantities between v.1 and v.2 version of the protocol;
D) Size distribution of fragments sequenced using v.1 (R1 and R2 are two independent repeats) and v.2 protocols from three patients using FFPE samples;
E) Distribution of signatures called from each of the samples in comparison with WGS data;
F) Number of mutations called by each method
G) Cosine similarity between WGS and indicated methods
H) Bar chart of cosine similarity between WGS and indicated methods

FIG. 10 shows a diagram of a comparison of the invention with WGS and WES.

FIG. 11 (sometimes referred to as FIG. 1.2/FIG. 4.1/FIG. 4/FIG. 4.1 of appendix A or FIG. 1.2 of Appendix B) shows sequence diagrams. Structural Comparison to Known Adapters. The figure provides the structure of standard known Illumina adapter (to the best of the inventors' knowledge) compared to the invention:
MutREAD=the invention.
quaddRAD=known adapter—for comparison only (Franchini et al 2017)
“standard Illumina adapter”=known adapter—for comparison only (Illumina Inc. (5200 Illumina Way, (formerly 5200 Research Pl), San Diego, CA 92122, USA). Oligonucleotide sequences for Illumina adapter(s) are © 2020 Illumina, Inc. All rights reserved.

The marking ‘may be’ against the 5′ phosphate of the standard Illumina adapter reflects a lack of disclosure if the phosphorylation is present in the known adapter as the inventor could not ascertain this from the documents available but to the best of the inventor's knowledge and belief the marked phosphate is thought to be present as it is required for ligations.

In more detail, the top images in FIG. 1.2 show sequences that are reverse complementary to SEQ ID NO: 3/SEQ ID NO: 4 in order to maintain the orientation and structure of libraries. Also, the binding site for at least one oligonucleotide primer has an additional T next to the UMI that is not in SEQ ID NO: 3. This T is included as Illumina uses T/A ligation in their library preps and this nucleotide, although not present in the sequences of the exemplary binding sites for at least one oligonucleotide primer such as SEQ ID NO: 3/SEQ ID NO: 4, it is introduced in the Illumina method after the Illumina ligation step (shown at the bottom of 1.2) and for this reason it is included in the binding site for at least one oligonucleotide primer in the exemplary adapter of the invention so as to ensure compatibility with sequence determination using Illumina NGS reagents.

FIG. 12 (sometimes referred to as FIG. 1.3) shows embodiments of the invention with the sticky ends present on the long strands (lower strands/bottom strands) of the adapters. The asterisk (*) shows where a single nucleotide in the (each) barcode sequence is changed relative to (i.e. different from) the recognition sequence of the relevant restriction enzyme to make it incompatible with the restriction enzyme site(s)—in this example the restriction enzymes are ApoI and PstI.

FIG. 13 (sometimes referred to as FIG. 1.4) illustrates how the invention may be implemented using ONT/Nanopore sequence determination technology.

FIG. 14 shows a table of nucleotide diversity for inner barcodes (sample barcodes)

FIG. 15 shows exemplary oligonucleotides

FIG. 16 shows diagrams

FIG. 17 shows plots which demonstrate that mutREAD allows for identification of relative copy number alterations in cancer cell line. In more detail, relative copy number alternations (CNA) were identified in the Flo-1 cell line using A) Whole Genome Sequencing (WGS) or mutREAD (B-D). WGS was performed at 30× coverage and mutREAD samples were sequenced to 100 million (100M) reads (equivalent of 110× in the mutREAD target regions and 7× genome-wide) and computationally down-sampled to C) 500 000 and D) 100 000 reads. CNAs in the WGS data were called using FREEC pipeline or using custom tools developed for mutREAD. CNAs were called at A) 10 kbp, B) 50 kbp, C) 500 kbp or D) 1000 kbp resolutions. Three regions (1-3) of focal CNA are marked with doted rectangle. The arrow indicates (D) indicates focal amplification captured at ultra-low coverage mutREAD.

### EXAMPLE 1

### Overview

We describe a cost-effective assay for quantifying mutational signatures in clinical cancer samples. Mutational processes acting on cancer genomes can be traced by investigating mutational signatures. High sequencing costs limit known approaches to small numbers of good-quality samples. We describe a robust, cost- and time-effective method, sometimes called mutREAD, to detect mutational signatures from small quantities of DNA, including degraded samples. We show that mutREAD recapitulates mutational signatures identified by whole genome sequencing, and enables the study of mutational signatures in larger cohorts and, by compatibility with formalin-fixed paraffin-embedded samples, in clinical settings.

We describe an easy-to-use method for mutational signature detection building on reduced representation sequencing (RR-seq) approaches that have been successfully applied in population genetics analyses. Our protocol is based on sequencing a reproducible, random subset of genomic regions generated by double-enzymatic digestion and subsequent fragment size-selection of the DNA sample. As a result, sufficient coverage for somatic mutation calling is achieved without bias in the type of detected mutations. The proposed method can detect mutational signatures from small quantities of DNA, including degraded samples from formalin-fixed paraffin-embedded (FFPE) material, in a robust, cost- and time-effective manner.

### Results

Our proposal assumes that obtaining a random subset of all mutations is sufficient to determine the presence of mutational signatures. To test this assumption, we first performed computational simulations (Methods) using available data from whole-genome sequencing of 129 esophageal adenocarcinoma (EAC) samples and the six mutational signatures derived from them¹³.

The stability of the mutational signature profile was evaluated as a function of the number of randomly selected mutations detected in the WGS samples (FIG. 1A). The cosine similarity relative to the original mutational signature profile increases with the number of mutations available for estimation. A plateau is reached at 500 mutations, suggesting that fewer than the WGS-derived number of mutations (on average 26 k mutations per EAC sample) are sufficient to obtain the mutational signature profile. The second assumption is that the mutation subset generated by RR-seq is an unbiased representation of the mutational spectrum. We simulated subsets of mutations for RR-seq using different enzyme combinations, as well as for 10× sWGS and WES (Methods). In this simulation, RR-seq with at least 161 out of 169 enzyme combinations outperforms (expanded) WES and 10× sWGS in terms of average cosine similarity between the WGS-derived and simulated signature profile in EAC (FIG. 1B). This difference can in part be attributed to the number of mutations recovered by the different methods (WES: 211, expanded WES: 282, 10× sWGS: 462 and RR-seq: 381 mutations on average). Notably, RR-seq derived mutations originate from a much lower proportion of the genome (a range of 0.2-82 Mbps, mean: 10 Mbps, 0.3% of WGS) than (expanded) WES-based mutations (WES: 46 Mbps/1.39% of WGS; expanded WES: 62 Mbps/1.88% of WGS).

We further investigated the applicability of RR-seq for estimating mutational signatures in different cancer types using the WGS data collected by the Pan-Cancer Analysis of Whole Genomes (PCAWG) network². RR-seq accurately estimated the mutational signature profiles across the majority of the 20 cancer types, including cancers with highly diverse mutational signature content, e.g. liver hepatocellular carcinoma (Liver HCC), and a non-solid tumor, i.e. B-cell non-Hodgkin lymphoma (Lymph-BNHL, Supplementary FIG. 1A). As expected from our simulations above, the performance of the method was correlated with the mutational load across cancer types (Supplementary FIG. 1B). Finally, RR-seq outperformed (expanded) WES in all cancer types:

Mutational signatures were computationally simulated across the PCAWG cohort. Summary of the cosine similarities (y-axis) of WGS-derived mutational signatures and mutational signatures derived from subsets of mutations simulating different sequencing approaches (x-axis) for each of the of individual tumor types from the PCAWG cohort. Boxes show the 25% and 75% quartile with the median across the samples indicated by the bold line. Whiskers extend to 1.5 times the interquartile range and samples outside this range are indicated as points. Different enzyme combinations were simulated for RR-seq, each shown as a different box. RR-Seq—reduced representation sequencing, WES—whole exome sequencing, expanded WES—whole exome sequencing expanded to untranslated regions and miRNAs.

(data not shown—Title of each page contains abbreviated tumor name (explained in supplementary FIG. 1) and the number of samples used for the analysis as follows: cosine similarity data for 20 different cancer types for studies from n11 to n271 subjects including Biliary-AdenoCA n34, Bone-Epith n11, Bone-Osteosarc n41, Breast-AdenoCa n110, CNS-Medullo n141, CNS-PiloAstro n89, Eso-AdenoCa n97, Head-SCC n13, Kidney-RCC n74, Liver-HCC n271, Lymph BNHL n98, Lymph-CLL n90, Myeloid-AML n16, Myeloid-MPN n51, Ovary-AdenoCA n69, Panc-AdenoCA n234, Panc-Endocrinen81, Prost-AdenoCA n256, Skin-Melanoma n70, Stomach-AdenoCA n32, available if required).

In addition All mutREAD data generated herein can be obtained from European Genome-phenome Archive. WGS data for the matched patient samples can be obtained from the ICGC data portal (https://dcc.icgc.org/). All analysis code can be obtained from https://github.com/jperner/mutREAD.

Having established superiority of RR-seq over other methods in the simulation, we implemented our approach, which we called mutREAD (Mutational Signature Detection by Restriction Enzyme-Associated DNA Sequencing), by changing, adapting and improving on the reagents and principles of the quaddRAD protocol²¹. Key features of the protocol include incorporation of Unique Molecular Identifiers (UMI) and inline barcodes, which allow for computational identification of PCR duplicates and larger multiplexing capabilities, respectively (FIG. 1C). The protocol is further streamlined by simultaneous enzymatic digestion and adapter ligation and removal of unnecessary purification steps. Here, we optimized the protocol towards application to EAC, for which six mutational signatures have been previously identified from WGS on fresh-frozen samples¹³. In particular, we chose the optimal pair of enzymes based on the simulation described above. The enzyme combination PstI and ApoI showed one of the highest cosine similarities to WGS results in EAC (FIG. 1B), as well as broad genome coverage and even distribution of target loci throughout the genome (FIG. 4—Supplementary FIG. 2). Hence, we designed adapter sequences that terminated with PstI and ApoI restriction enzyme compatible sites and that are devoid of PstI or ApoI restriction enzyme sites to avoid digestion of the adapters (Supplementary Table 1).

We further optimized the protocol to suit either fresh-frozen or FFPE samples (Methods), the latter being the standard sample preservation strategy in clinical practice. Restriction enzyme double digestion, adapter ligation conditions and size selection were optimized for optimal digestion, adapter annealing and size selection using an EAC cell line (FLO-1). The protocol was further adjusted for FFPE derived DNA from the same EAC cell line (FIG. 5—Supplementary FIG. 3).

We then applied mutREAD to fresh-frozen tumor and matched blood samples from biopsies of three different EAC patients and evaluated the quality of the library under several criteria (Supplementary Table 2, FIG. 6—Supplementary FIG. 4). The mutational signatures, derived from 530-1471 mutations detected using GATK Mutect²³, showed cosine similarities of 0.95-0.96 when compared with the WGS-derived mutational signature profiles (FIG. 1D). We observed similar cosine similarity between mutREAD and WGS when mutations were derived using an alternative mutation caller, Strelka²⁴ (Supplementary Table 3). In summary, the mutREAD protocol results in reproducible, good quality, target-specific libraries from which mutational signatures can be successfully derived.

Next, we compared mutREAD with WES and 10× sWGS libraries of the same samples sequenced to similar depth. Quality measures for the resulting libraries of the different methods are summarized in Supplementary Table 4. WES resulted in 46-325 mutations per sample and 10× sWGS identified 21-83 mutations per sample. mutREAD consistently achieved high cosine similarity to the corresponding WGS-derived signatures. Conversely, WES and 10× sWGS had lower cosine similarities and much higher variability between patients (FIG. 1D).

Finally, we investigated if mutREAD can be used to study historical samples by sequencing FFPE specimens matching the previously analyzed frozen samples. Fresh frozen and FFPE-derived samples generated similar signature patterns (FIG. 2A), despite the lower sequencing depth and smaller fragment distribution of final FFPE-derived libraries (FIG. 6—Supplementary FIG. 4, Supplementary Table 2). Cosine similarities to WGS-derived mutational signatures were between 0.89-0.96 based on 47-383 detected mutations.

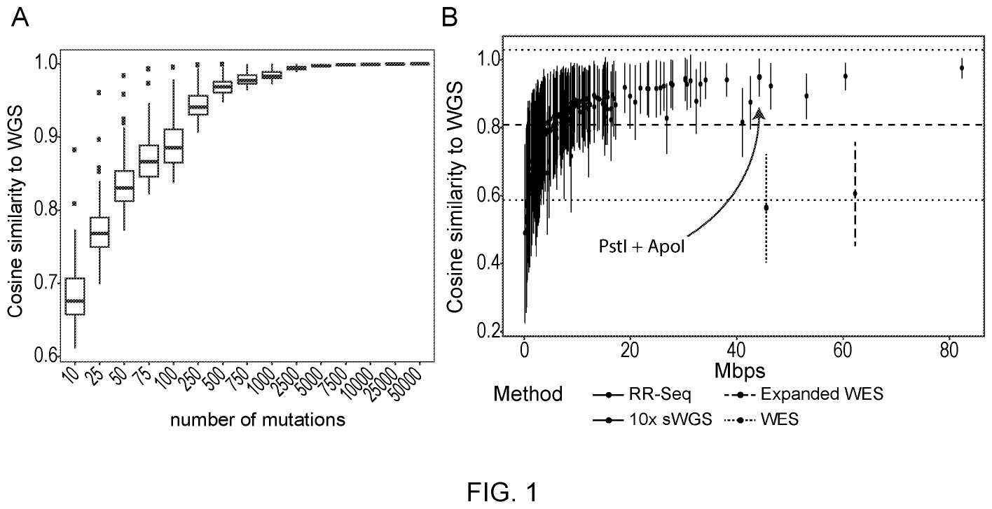

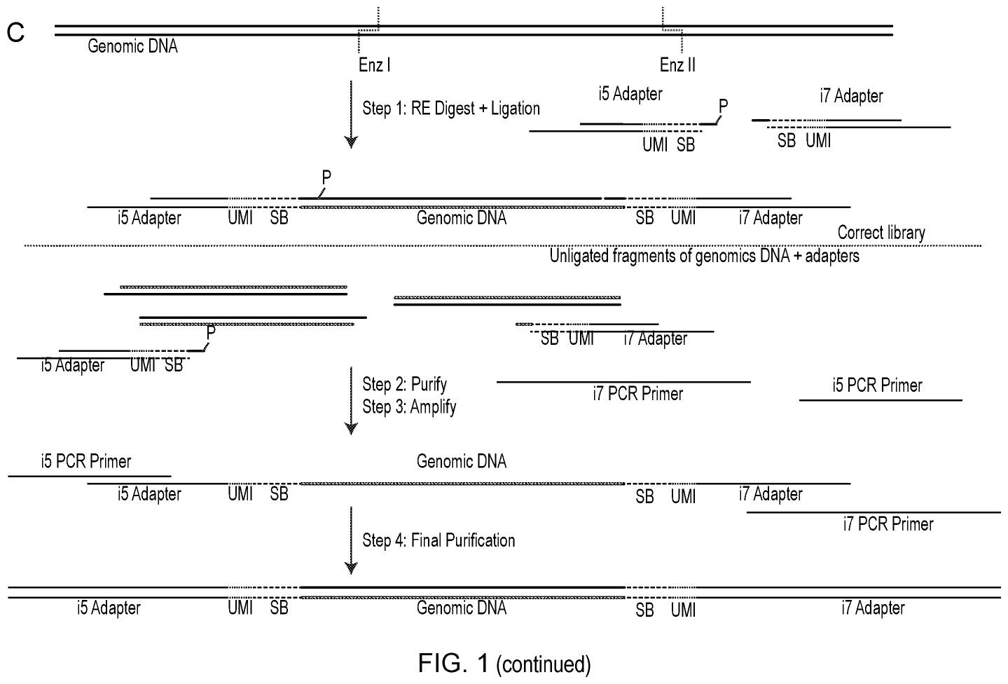

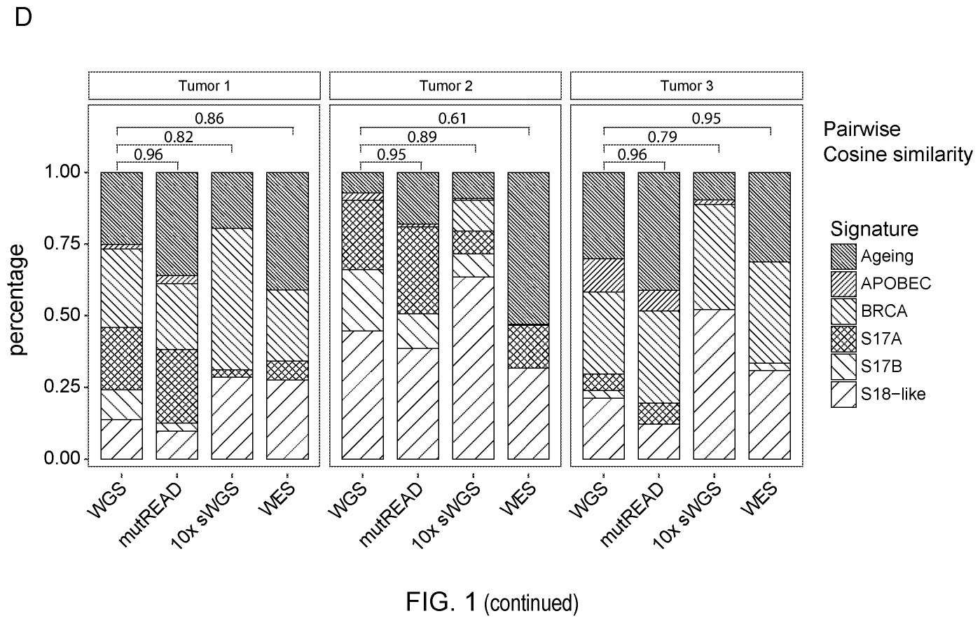

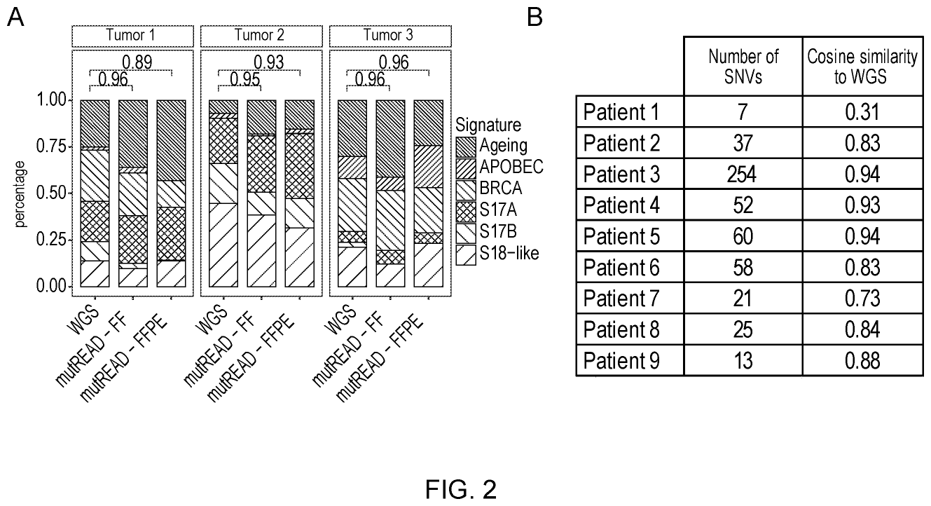

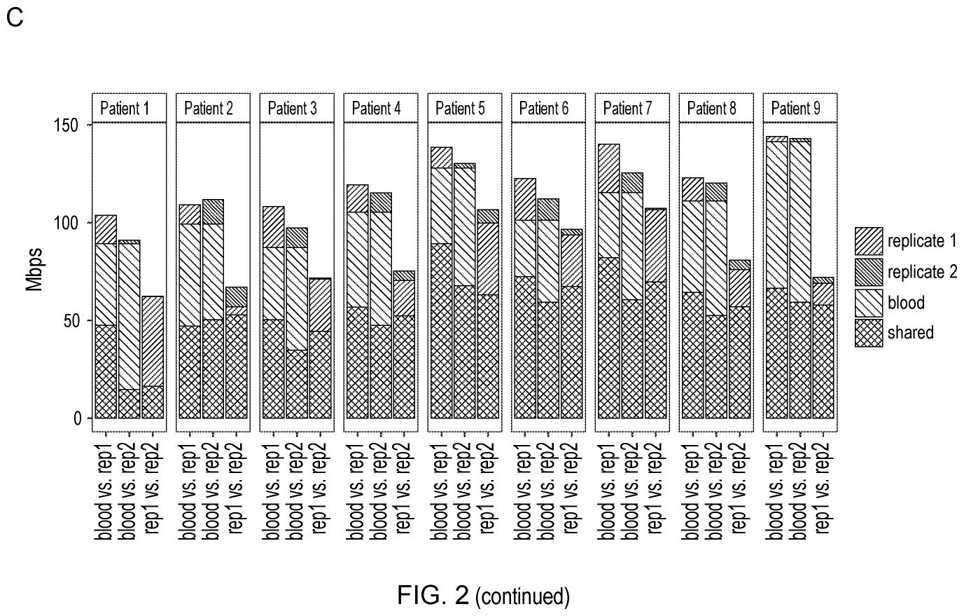

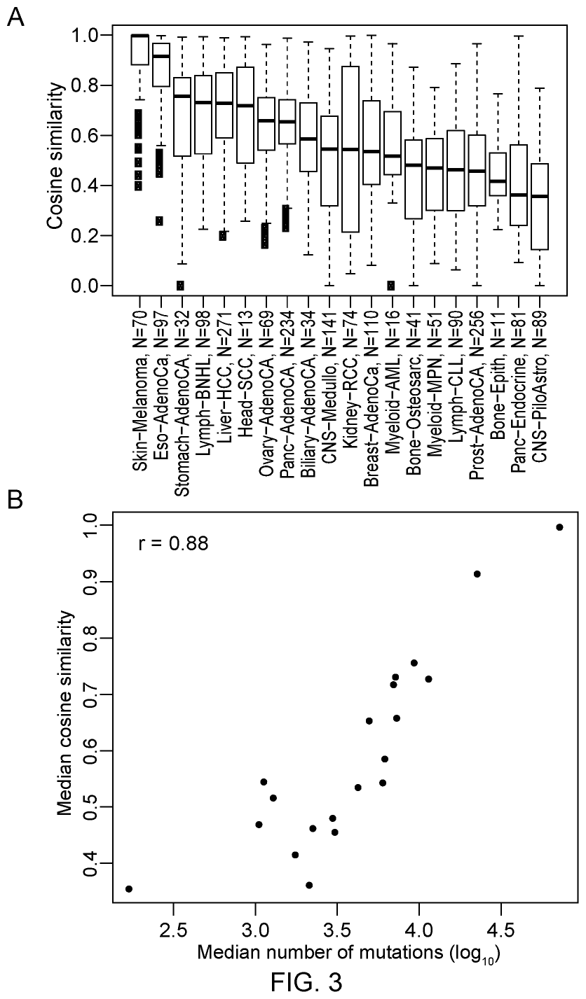

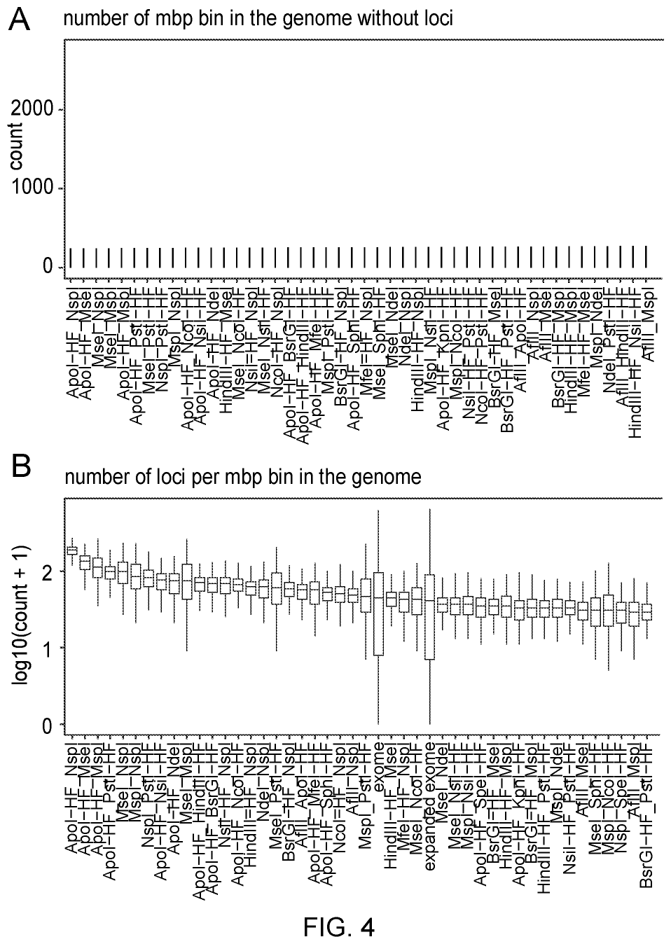

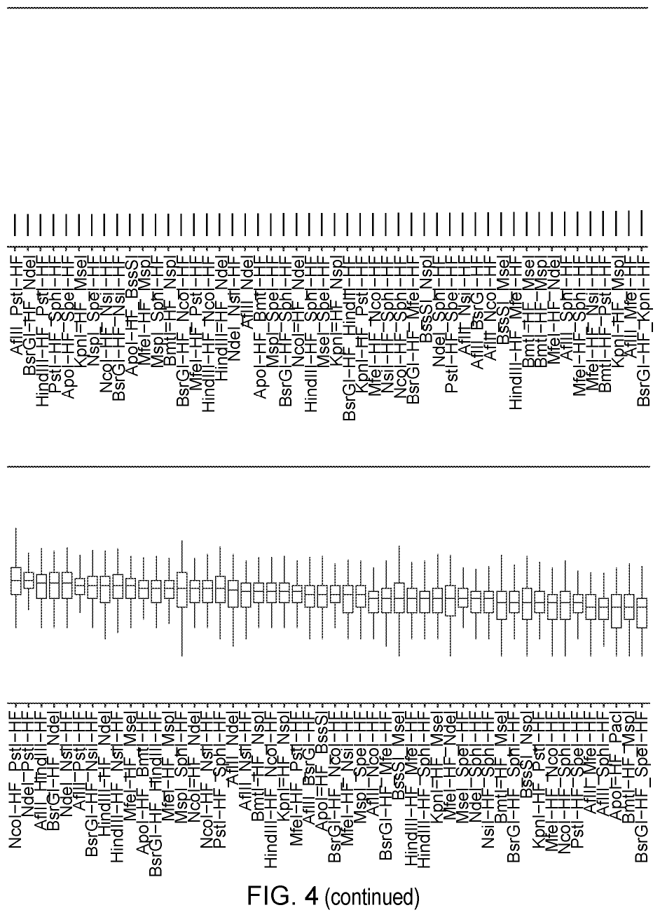

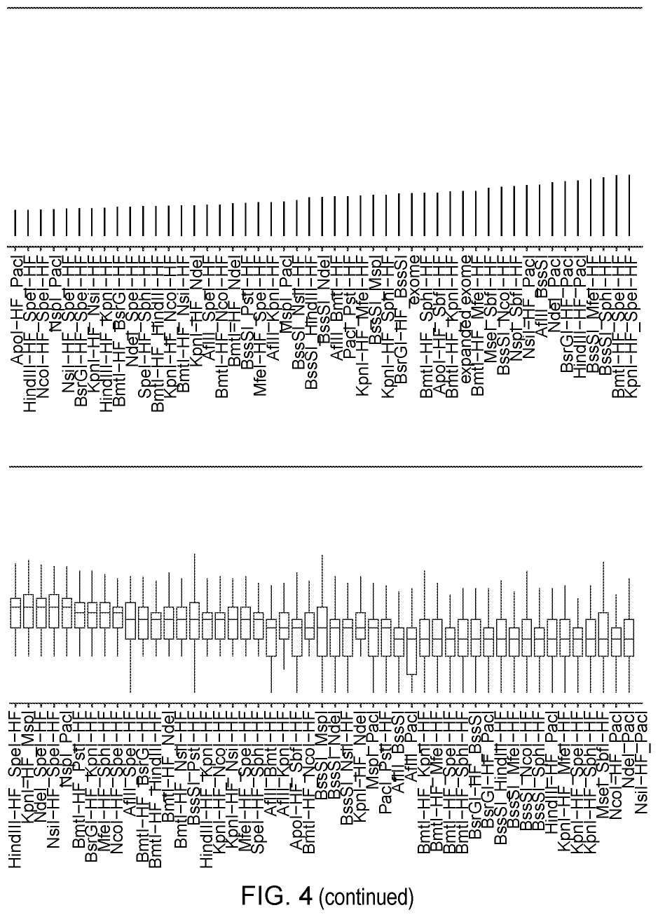

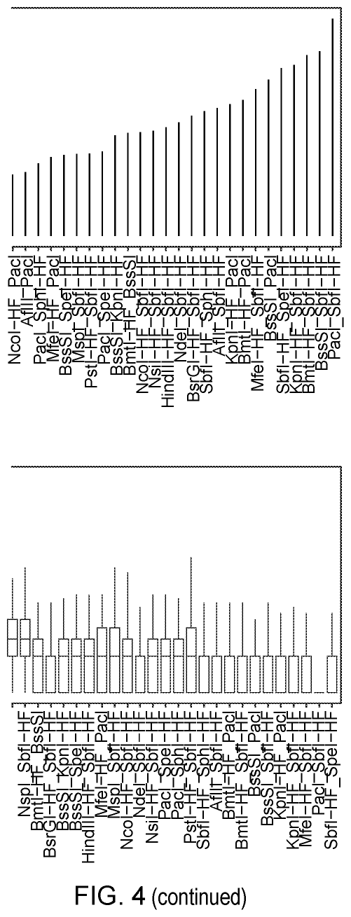

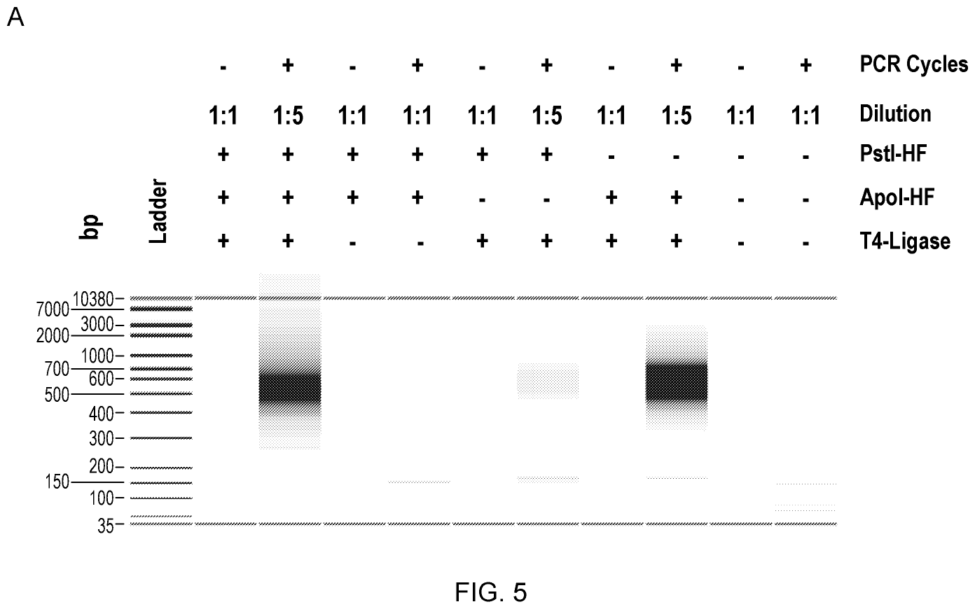

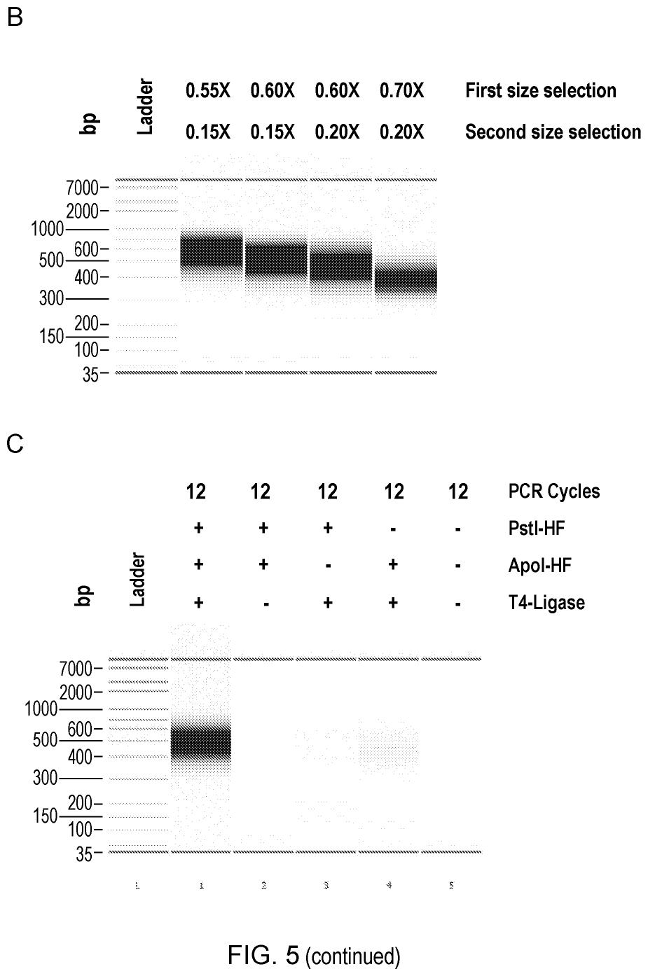

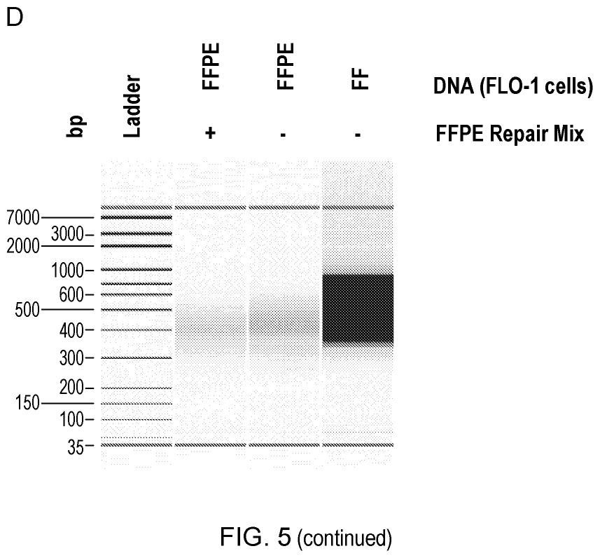

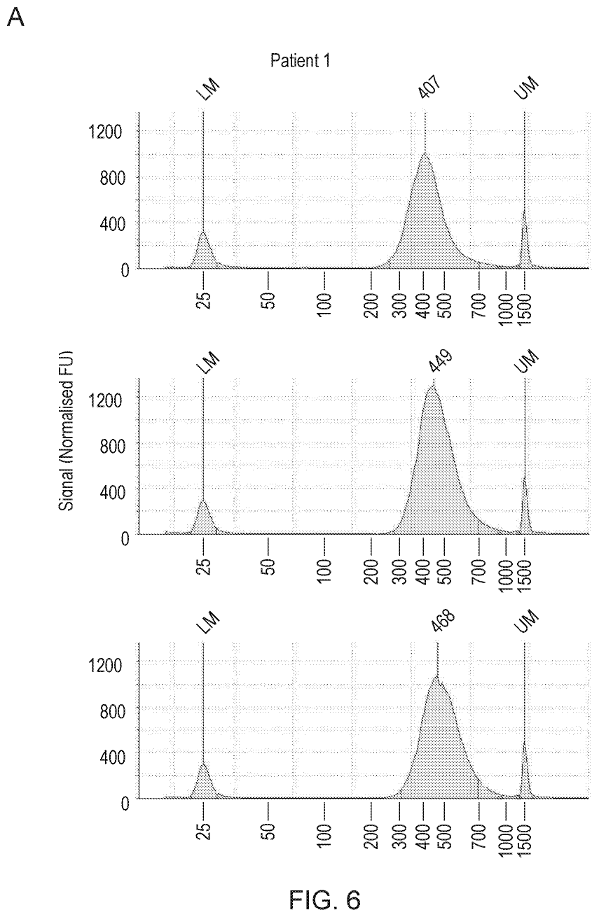

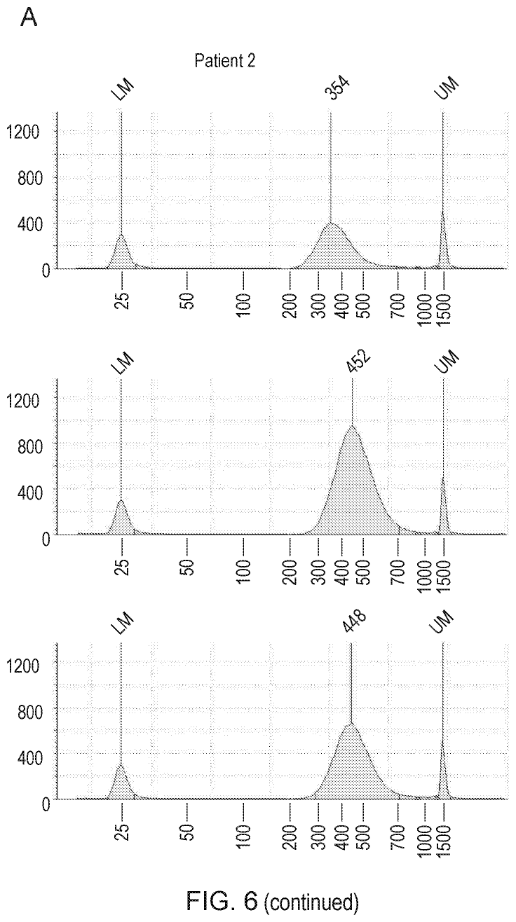

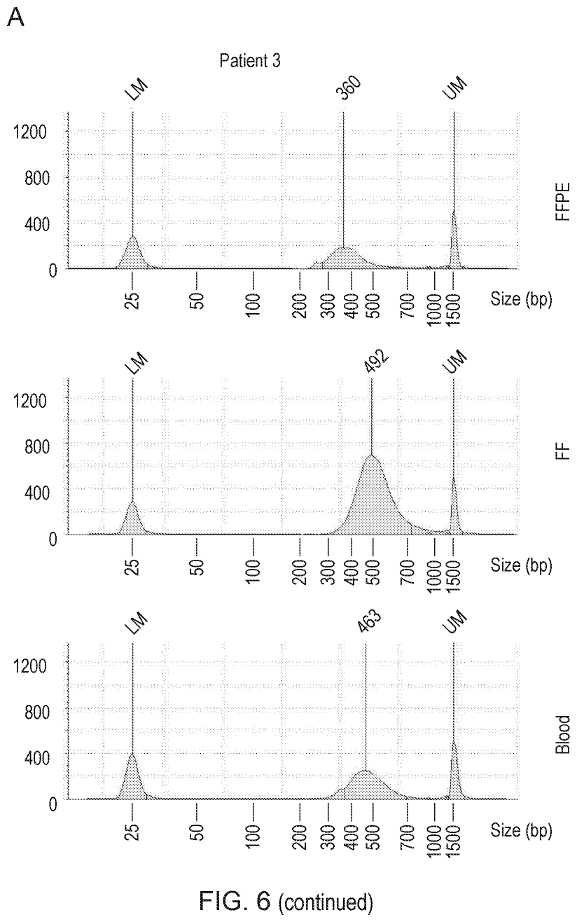

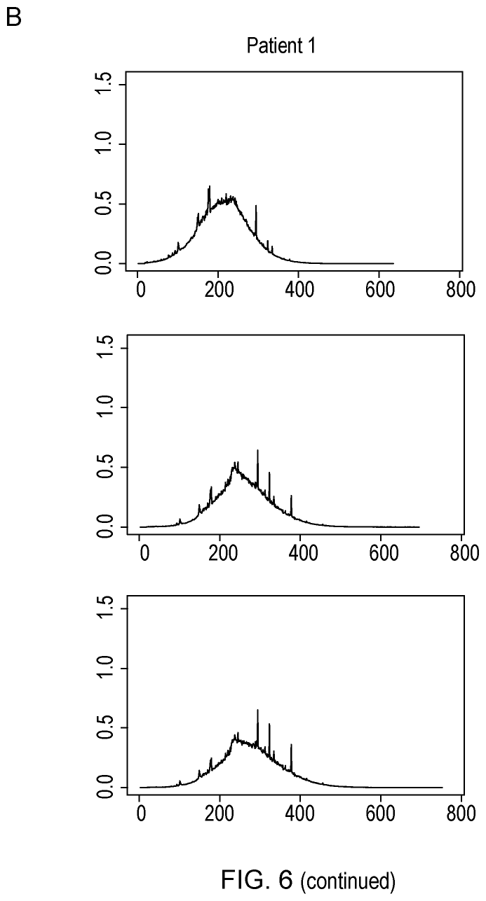

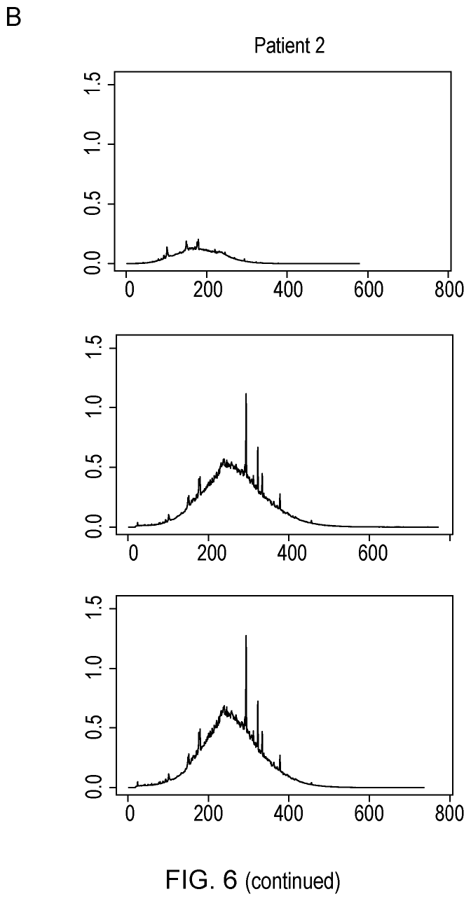

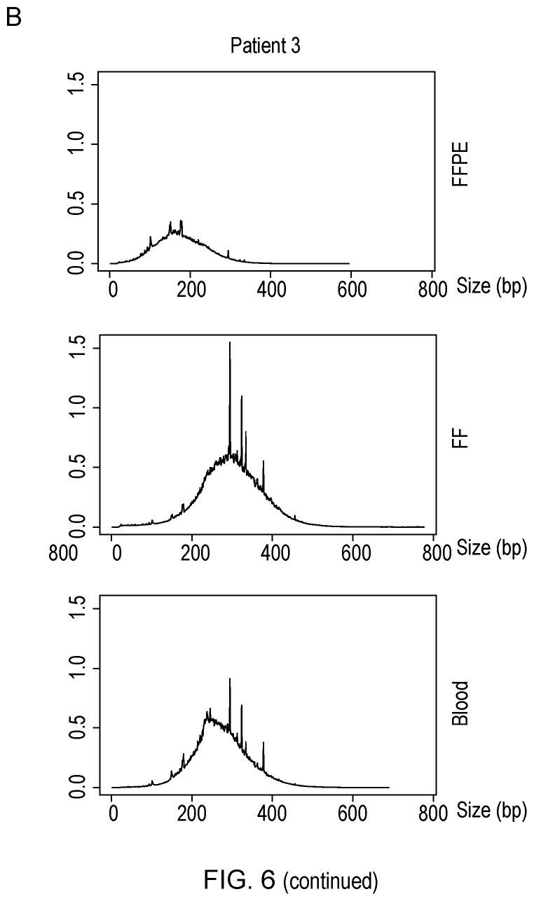

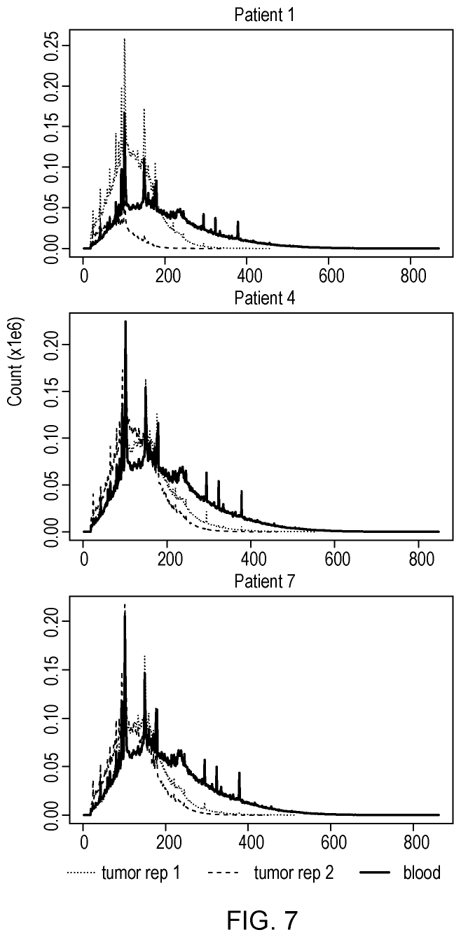

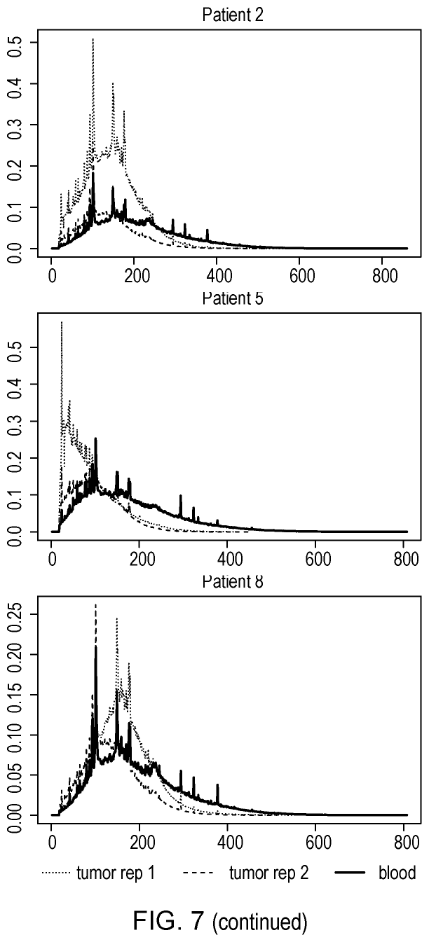

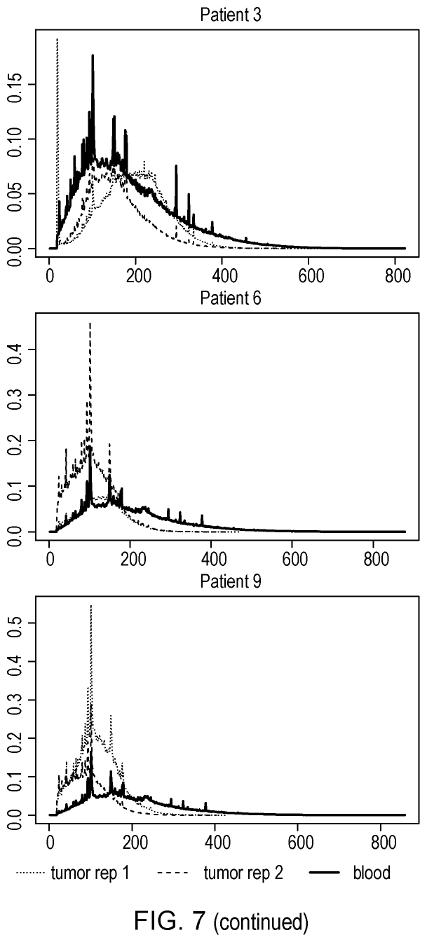

We replicated the good cosine similarity to WGS-derived mutational signatures in additional nine FFPE samples (FIG. 2B). Of note, samples were derived from tumor resections and pathology estimates for these samples show low tumor content (10-70%, Supplementary Table 5), explaining the lower number of mutations and higher variability across samples compared to the previously tested biopsy samples. Given the high degradation expected in FFPE samples which can result in variability, we also tested the reproducibility of FFPE-derived mutREAD libraries. Technical replicates of the nine FFPE samples showed high concordance in sequenced regions and fragment size distribution (FIG. 2C, FIG. 7—Supplementary FIG. 5). Hence, while it is expected that the performance on FFPE is lower compared to fresh-frozen samples, our results suggest that mutREAD can also be applied to FFPE-derived DNA samples with low tumor content and leads to reproducible results.

### Methods

### Enzyme Selection Criteria

The enzyme combination is an important parameter to optimize for the mutREAD method. We focused on high-fidelity restriction enzymes provided by New England BioLabs Inc. (Ipswich, Massachusetts USA) to allow for fast DNA digestion and maximum target specificity under a broad range of experimental conditions. Since cancer samples frequently exhibit DNA hyper- or hypo-methylation, which could affect restriction enzyme sites, we required insensitivity to CpG methylation status. To simplify the adapter design, only enzymes with a unique cut-site including only A, C, G and T were considered. Finally, cut sites were required to have a maximum length of six base pairs to increase the number of generated fragments. The tested list of enzymes is given in Supplementary Table 7.

### Simulations

We opted for a double-digest protocol to produce fragments that are reproducible between libraries. To simulate the performance of all possible enzyme combinations full-filling the above criteria, we use ddRADseqTools (v0.45)²⁸ to perform in silico digestion of the human hg19 reference genome and size selection for fragments of expected length between 350-450 bp. The expected fragment size range of 350-450 base pairs was chosen as the maximum fragment size such that the complete library fragments (insert, adapters and primers) could still be sequenced on a standard Illumina HiSeq system. WGS-based mutations were selected if they overlap the resulting expected fragments and mutational signatures were calculated based on this selection. Similarly, WES and expanded WES sequencing is simulated using the target regions provided by Nextera for the rapid capture exome/expanded exome kit (v1.2)²⁹, where the exome kit comprises 45 Mbps of coding regions and the expanded exome kit comprises 62 Mbps of coding regions, untranslated regions and miRNAs. Further, the 21 simulated 10× sWGS libraries from a previous study¹³ were used. In short, the 10× sWGS were simulated by down-sampling the WGS libraries and re-running the mutational calling.

### Cosine Similarity

We measure similarity between two mutational signature profiles P and Q using the cosine similarity. The cosine similarity between the non-zero vectors P and Q with n mutational signatures is defined a

$$cos sim ( P , Q ) = \frac{{\sum }_{i = 1}^{n} {P}_{i} {Q}_{i}}{\sqrt{{\sum }_{i = 1}^{n} {P}_{i}^{2}} \sqrt{{\sum }_{i = 1}^{n} {Q}_{i}^{2}}} .$$

Two mutational signature profiles that are independent have cosine similarity of 0. Conversely, identical mutational signature profiles obtain a cosine similarity of 1.

Computational simulations using Pan-Cancer Analysis of Whole Genomes data We also performed computational simulations on the WGS data from the PCAWG network. The collection was downloaded from https://dcc.icgc.org/releases/PCAWG/consensus_snv_indel. We have used the signature compendium from COSMIC (v3, downloaded from https://dcc.icgc.org/releases/PCAWG/mutational_signatures/Signatures/SP_Signatures/SigProffler_reference_signatures) to capture all mutational signatures relevant to the different cancer types. Only cancer types with at least 10 samples present in the collection were analyzed.

### Ethical Approval, Sample Collection

Esophageal adenocarcinoma samples were collected by the Oesophageal Cancer Classification and Molecular Stratification (OCCAMS) project, a multi-center UK-wide study. The study was approved by the Institutional ethics committee (REC 07/H0305/52 and 10/H0305/1) and included individual informed consent.

### Assay Optimization

All optimization experiments were performed using 500 ng of genomic DNA from an EAC cell line (FLO-1), commercially available from culture collection of Public Health England. In-house STR analysis was done in the lab to confirm a >90% match prior to assay optimization. Experiments were then repeated with frozen tumor, matched blood and FFPE tumor DNA from EAC patients.

### DNA Extraction and Quantification

DNA was extracted from FLO-1 cell line and frozen tumors using the Allprep DNA/RNA mini kit (Qiagen, Hilden Germany) and DNA from blood was isolated using QIAmp DNA blood maxi kit (Qiagen, Hilden Germany). AllPrep DNA/RNA FFPE Kit (Qiagen, Hilden Germany) was used to extract DNA from FFPE tumors. DNA quantification was done using Qubit dsDNA Broad Range (BR) assay kit on Qubit 3.0 fluorometer (Thermo Fisher Scientific, Waltham Massachusetts USA).

Restriction digestion optimization for ApoI HF-PstI HF double digest High-Fidelity (HF) ApoI and PstI restriction enzymes were obtained from New England BioLabs Inc. (Ipswich, Massachusetts USA). The optimization of restriction enzyme digestion (Supplementary FIG. 4) was performed on 500 ng of FLO1 cell line genomic DNA and included optimization of enzyme concentration, library purification procedure, PCR cycle optimization and removal of FFPE artefacts.

### Adapter Design and Primers

Adapters (i5 and i7, Supplementary Table 1) were designed to target DNA fragments with restriction overhangs for the selected restriction enzymes (PstI and ApoI) and achieve specific and uniform sampling of the genome by modifying Illumina adapter sequences³⁰ following the general principles of the quaddRAD protocol²¹. The random 4 bp degenerate barcode included in both, i5 and i7, was designed to avoid creating new restriction sites. The 6 bp unique inner barcode sequences were balanced for A/C and G/T content to increase the sequence diversity at each position across the inner barcodes. Additionally, PhiX control was spiked in to 20% to improve the overall sequencing quality. The upper strand of the first adapter was phosphorylated to abolish the ligation at the 3′ end and the lower strand of the first adapter was phosphorylated for its ligation with the DNA insert. To avoid non-specific amplification during the PCR stage the i7 adapters were designed in a Y-shape conformation to amplify only those DNA fragments with specific adapters ligated to them. Illumina universal PCR primers (i5nn and i7nn) were used for amplification (Supplementary Table 1). A phosphorothioate bond at the 3′ end of the outer barcodes/primers (i5nn/i7nn) was added to protect from nonspecific or proofreading nuclease degradation.

### Adapter Preparation

Lyophilized adapters obtained from Integrated DNA Technologies (IDT, Leuven Belgium) were reconstituted in Tris-EDTA (TE pH:8) buffer to get 100 μM stock. Complementary upper and lower single strands of i5 and i7 were annealed at 10 μM each using annealing buffer (500 mM NaCl, 100 mM Tris-HCl, pH 7.5-8) on a thermal cycler with the following conditions: Denature at 97.5° C. for 2.5 min and then bring down to 4° C. at a rate of 3° C./min. Hold at 4° C. Adapters were stored in −20° C. This 10 μM working dilution of adapters stock was used in ligation reaction.

### Library Preparation and Sequencing

Double Restriction digestion and ligation reaction: Both restriction digestion and ligation reaction were performed simultaneously. Soong of genomic DNA was digested with 50 U of PstI-HF and ApoI-HF in presence of 0.187 mM first and second oligonucleotide adapters of the invention (referred to here as mutREAD i5 and i7 adapters respectively), 400 U of T4 ligase and 1 mM ATP in 1× CutSmart buffer. The reaction was incubated on a thermal cycler at 30° C. for 3 hours. Ligation reaction was stopped by addition of 10 μl of 50 mM EDTA.

Size selection: Two step size selection for 400-500 bp inserts (DNA fragments, excluding adapters) was performed using Agencourt AMPure XP beads (BECKMAN COULTER, Brea California US). Unwanted larger fragments were removed with 0.6× ratio of AMPure beads to ligation product and the short fragments were removed by 0.15× size selection.

PCR Amplification of Library: The size selected DNA fragments ligated with adapters (20 l) were amplified using PCR primers (i5nn/i7nn) compatible with Illumina sequencing platform. The reaction was performed in total volume of 100 μl with 0.8 U of Phusion high-fidelity polymerase, in the presence of 0.2 mM dNTPs and 1×Phusion High Fidelity buffer. PCR was performed in the following conditions: 98° C./2 min denaturation, 12 cycles of amplification at 98° C./10 sec, 65° C./30 sec, 72° C./30 sec and final extension at 72° C. for 5 min. Libraries were purified using 0.8× AMPure beads (80 μl beads+100 μl library), this step was repeated one more time to remove all unwanted leftover reactants during PCR. Libraries were eluted in 20 μl TE buffer (Tris-EDTA buffer 10 mM TrisHCl and 0.1 mM EDTA, pH8) and stored at −20° C. Quality control was performed on Agilent 2100 Bioanalyzer using Agilent High Sensitivity DNA kit (Santa Clara, California, US) or High Sensitivity D1000 TapeStation kit (Agilent). Quantification of the libraries was performed using KAPA Library Quantification kit (KK4953-07960573001 for Illumina platforms, Kapa Biosysytems Roche Holding AG Basel Switzerland) on the Light cycler 480 (Roche Life Sciences, Basel Switzerland). Libraries with unique adapters were pooled and sequenced on the HiSeq4000 using paired end, 150 bps chemistry.

### De-Multiplexing and PCR Duplicate Identification

After sequencing, all libraries were de-multiplexed using the outer barcodes. Next, for libraries containing random/degenerated molecular barcodes, PCR duplicates were identified and removed using Stacks' clone_filter (version 1.46)³¹, allowing for random oligos of length 4 bp at both ends of the read pair. Another round of de-multiplexing using all possible combinations of inner barcodes, low quality read filtering and filtering of reads without the appropriate RAD-tag was performed with Stacks' process_radtags.

### Read Mapping and Quality Metrics

The final libraries were mapped to the hg19 human reference genome (GRCh37_g1k) using BWA MEM (0.7.15)³². Resulting same files were converted to bam, sorted and indexed using samtools (1.3.1)³³. Quality metrics were calculated using GATK callableLoci (v3.7-0) for identifying loci with at least 10× coverage, Picard (2.9.0)³⁴ CollectInsertSizeMetrics to calculate fragment size histograms from mapped read pairs, and samtools flagstat to obtain mapping statistics.

### Somatic Mutation Calling

Mutation calling was performed using GATK Mutect²³, taking into account for the SNV metrics only reads with minimum mapping quality of 1, minimum base quality of 10 and excluding supplementary alignments, as well as discarding both reads in an overlapping read pair if they have different base calls at the locus of interest, or using just the read with highest base quality if they have the same base.

Additionally, Strelka (v 2.0.15) with disabled read depth filter was run on a subset of samples, taking into account for the SNV metrics only reads with minimum mapping quality of 1, minimum base quality of 10 and allowing a minimum alternate allele count of 2 and a minimum alternate allele frequency of 0.05 for a position to be considered in detecting SNV clusters.

For Mutect2- and Strelka-derived mutations, low-quality and spurious mutation calls were filtered by applying the following criteria¹³: VariantAlleleCountControl>1, VariantMapQualMedian<40.0, MapQualDiffMedian<−5.0∥MapQualDiffMedian>5.0, LowMapQual>0.05, VariantBaseQualMedian<30.0, VariantAlleleCount>=7 && VariantStrandBias<0.05 && ReferenceStrandBias>=0.2. The parameter ReadCountControl was set to be <20 for the three fresh-frozen and FFPE paired samples and <10 for the additional FFPE samples.

Additionally, based on the cosine similarity of WGS-derived mutational signatures and the mutational signatures derived for the initial three samples, we optimized the minimum number of reads supporting a SNV (fresh-frozen samples mutREAD=5, WES=7, 10× sWGS=5, mutREAD FFPE=10) and the minimal variant allele frequency of a SNV (fresh-frozen samples mutREAD=0.03, WES=0.01, 10× sWGS=0.11, mutREAD FFPE=0.13). The cut-offs were optimized separately for Strelka-derived mutations (fresh-frozen samples=20 reads and 0.11 variant allele frequency, mutREAD FFPE=11 and 0.03 variant allele frequency).

### Mutational Signature Profile

The tri-nucleotide context for each SNV was determined using the SomaticSignatures R package³⁵. Mutational signature profiles were derived for each sample using EAC-specific mutational signatures¹³. Finally, non-negative least squares in R was used to derive the contributions of each mutational signature to the overall mutational spectrum. The estimated coefficients were scaled to sum up to one.

### Discussion of Example 1

We have described the development and application of a cost-effective and scalable method for the detection of mutational signatures in DNA samples. mutREAD produces reproducible and highly specific reduced representation libraries and the derived mutational signatures mirror the WGS-derived signatures with high cosine similarity. Importantly, this also holds true even when used with highly degraded DNA samples. Our method enables the study of mutational signatures in much larger cohorts and in clinical settings where FFPE-derived DNA samples are routinely collected. Applied to tumor samples from EAC patients, we show that the invention (‘mutREAD’) outperforms the known methods WES and 10× sWGS. EAC is characterized by abundant somatic mutations, which are most prevalent in intergenic and intronic regions^{13,25}. The choice of library preparation methods to study mutational signatures in other cancer types will depend on the overall mutation rate and the genomic distribution of the somatic mutations.

In terms of scalability and cost the invention (‘mutREAD’) outperforms known methods (Supplementary Table 6). In our hands, the cost associated with mutREAD libraries synthesis is 80% lower than for 10× sWGS and 96% lower than for WES libraries. Sequencing costs on the Illumina HiSeq 4000 are comparable for WES and mutREAD libraries, while sequencing 10×WGS libraries is at least three times more expensive. Further, due to its high multiplexing capabilities for sequencing and for library preparation mutREAD is highly scalable for studying larger cohorts.

Given its ease of use and low cost, the invention finds utility and industrial application in wide range of applications to study mutational signatures in basic research and translational settings. For example, clinical trials using mutational signature-based patient stratification to assign optimal therapies become feasible. The invention can further improve the mutational signature-based prediction of homologous recombination deficiency in clinical samples^{14,26}. Together with computational tools for coarse-grained copy alteration detection^{22,27}, the invention could provide a detailed view of the role of mutational processes in cancer progression and evolution from archived material. Finally, correlative analyses of mutational signatures with endogenous and environmental parameters to understand the source of so far unknown mutational signatures will shed light on the etiology of cancers.

SUPPLEMENTARY TABLE 1
A) mutREAD adapter sequences

| Adapter | Adapter name | Sequence |
|---|---|---|
| mutREAD- | mutREAD-i5-upper_1_ATGAGCGA | 5′-C GCT CTT CCG ATC T HNNNATGAGCGATGCA-phos-3′ SEQ ID NO: 13 |
| i5 | mutREAD-i5-lower_1_TCGCTCAT | 5′-phos-TCGCTCATNNNDA GAT CGG AAG AGC GTC GTG TAG GGA AAG AGT |
| | | GT-3′ SEQ ID NO: 14 |
| | mutREAD-i5-upper_2_GCCTAGCG | 5′-CGCTCTTCCGATCTHNNNGCCTAGCGTGCA-phos-3′ SEQ ID NO: 15 |
| | mutREAD-i5-lower_2_CGCTAGGC | 5′-phos-CGCTAGGCNNNDAGATCGGAAGAGCGTCGTGTAGGGAAAGAGTGT-3′ SEQ |
| | | ID NO: 16 |
| mutREAD- | mutREAD-i7-upper_1_CGTGTACC | 5′-GTGACTGGAGTTCAGACGTGTGCTCTTCCGATCTHNNNCGTGTACC-3′ SEQ ID NO: 17 |
| i7 | mutREAD-i7-lower_1_GGTACACG | 5′-AATTGGTACACGNNNDAGATCGGAAGAGCA-3′ SEQ ID NO: 18 |
| | mutREAD-i7-upper_2_GCACATGT | 5′-GTGACTGGAGTTCAGACGTGTGCTCTTCCGATCTHNNNGCACATGT-3′ SEQ ID NO: 19 |
| | mutREAD-i7-lower_2_ACATGTGC | 5′-AATTACATGTGCNNNDAGATCGGAAGAGCA-3′ SEQ ID NO: 20 |

Legend
NNNNN Unique Molecular Identifier
NNNNN Inner sample barcode
NNNNN Outer sample barcode
B) mutREAD primer sequences

| Primer | Primer Name | Sequence |
|---|---|---|
| mutREAD- | mutREAD-i501_TATAGCCT | 5′-AATGATACGGCGACCACCGAGATCTACACTATAGCCTACACTCTTTCCCTACACGAC*G-3′ |
| i5nn | | SEQ ID NO: 21 |
| | mutREAD-i502_ATAGAGGC | 5′-AATGATACGGCGACCACCGAGATCTACACATAGAGGCACACTCTTTCCCTACACGAC*G-3′ |
| | | SEQ ID NO: 22 |
| mutREAD- | mutREAD-i701_ATTACTCG | 5′-CAAGCAGAAGACGGCATACGAGATCGAGTAATGTGACTGGAGTTCAGACGTGTGC*T-3′ |
| i7nn | | SEQ ID NO: 23 |
| | mutREAD-i702_TCCGGAGA | 5′-CAAGCAGAAGACGGCATACGAGATTCTCCGGAGTGACTGGAGTTCAGACGTGTGC*T-3′ |
| | | SEQ ID NO: 24 |

mutREAD adapters and primer
A) Summary of the sequences of mutREAD adapters used for ligation to DNA fragments. Color of the
nucleotides indicates specific elements of the adapters (unique molecular identifiers, inner samples
barcodes). Ambiguous base codes H and D translate to bases A/C/T and A/G/T, respectively. Adapter
names include the arm of the adapter (in this example the first adapter (‘i5’) contains PstI
compatible end and the second adapter (‘i7’) - ApoI compatible end) and the sequence of the samples
barcode.
B) Summary of the Illumina compatible primers used for amplification of the ligated libraries. Color
indicates the sequences and location of outer sample barcode. Adapter names include the arm of the
primer (i5XX is compatible with first adapter (‘i5 adapter’) and i7XX with second adapter (‘i7
adapter’) and the sequence of the samples barcode.
Abbreviations: phos - phosphorylation of the indicated nucleotide, * - phosphorothioate bond between
the indicated bonds.

| | | | | | | Percent of | | | |
|---|---|---|---|---|---|---|---|---|---|
| | | | | | | reads lost | | | |
| | | Number of | | Percent lost | | due to | Percent | Estimated | Base pairs |
| | | reads after | Percent | due to | Percent | inner | lost due to | average | covered |
| Patient | Sample | outer barcode | retained | unidentifiable | lost due to | barcode | ambiguous | fragment | with at |
| ID | type | demultiplexing | after QC | barcode | low quality | mixup | RAD-tag | size (bp) | least 10x |
| 1 | FF | 158,178,068 | 94.54 | 0.09 | 0.37 | 3.83 | 1.17 | 260 | 175,049,803 |
| | FFPE | 184,526,290 | 93.41 | 0.33 | 0.36 | 4.46 | 1.43 | 215 | 170,810,606 |
| | Blood | 155,543,840 | 94.14 | 0.07 | 0.42 | 3.52 | 1.85 | 276 | 186,266,055 |
| 2 | FF | 206,790,834 | 92.68 | 0.23 | 0.62 | 5.55 | 0.91 | 263 | 195,958,931 |
| | FFPE | 43,949,748 | 94.92 | 0.23 | 0.47 | 3.41 | 0.98 | 183 | 95,105,098 |
| | Blood | 230,847,264 | 96.21 | 0.13 | 0.48 | 2.37 | 0.80 | 257 | 193,634,665 |
| | FF | 231,185,296 | 95.00 | 0.07 | 0.46 | 3.56 | 0.92 | 297 | 198,984,001 |
| 3 | FFPE | 86,259,612 | 94.26 | 0.67 | 0.59 | 3.33 | 1.15 | 178 | 131,586,722 |
| | Blood | 194,066,264 | 96.39 | 0.08 | 0.47 | 2.02 | 1.05 | 273 | 190,613,393 |

| | Percent of | Base pairs in | | | | |
|---|---|---|---|---|---|---|
| | retained | 10x loci shared | | | | |
| | reads | in | Base pairs | Base pairs | mutREAD- | WGS- |
| Patient | contributing | tumour/blood | covered with at | covered with | Number of | Number of |
| ID | to 10x loci | pair | least 50x | at least 100x | mutations | mutations |
| 1 | 96.54 | 166,936,824 | 98,935,164 | 60,297,417 | 1,050 | 28,732 |
| | 96.91 | 143,331,473 | 122,274,170 | 86,782,166 | 383 | |
| | 96.26 | — | 103,044,362 | 60,323,363 | — | — |
| 2 | 96.63 | 187,858,494 | 147,765,532 | 105,375,328 | 1,471 | 27,764 |
| | 93.98 | 88,953,041 | 32,115,195 | 10,201,299 | 47 | |
| | 96.61 | — | 147,906,131 | 111,689,924 | — | — |
| 3 | 96.57 | 170,614,310 | 146,079,968 | 106,880,654 | 530 | 11,068 |
| | 95.11 | 113,854,654 | 77,474,870 | 36,663,830 | 90 | |
| | 96.49 | — | 114,092,331 | 73,822,016 | — | — |

Supplementary Table 2-Quality metrics for mutREAD libraries derived from tumor, FFPE and blood samples of three patients.
The table summarizes quality metrics for each sample, including fresh-frozen (FF) and formalin-fixed paraffin-embedded (FFPE) tumor, as well as blood samples (column 2) from three patients (column 1).
Sample groups of three were sequenced on one lane, where each sample had a unique outer barcode.
Number of reads derived from the libraries de-multiplexed by outer barcode are listed in column 3.
Percentages (with respect to column 3) of reads that are retained for further analysis (column 4) or filtered due to an unidentifiable inner barcode (column 5), low read quality (column 6), wrong/unexpected inner barcode (column 7), missing restriction site overhang (column 8) are listed in the respective columns.
The average fragment size derived from read pair mates after mapping is given in column 9 (related to Supplementary FIG. 4).
The number of base pairs covered with at least 10x, 50x and 100x is listed in column 10, 13 and 14, respectively.
The percentage of retained reads (column 4) contributing to loci defined in column 10 is given in column 11.
Finally, the overlap between tumor and blood samples in loci defined in column 10 is shown in column 12.
The number of mutations used for deriving the mutational signatures is given in column 15 and 16.

A) Number mutations (fresh-frozen)

| | mutREAD | | | WGS | | |
|---|---|---|---|---|---|---|
| | Mutect2 | Strelka | Consensus | Mutect2 | Strelka | Overlap |
| Tumor1 | 1050 | 520 | 440 | 28732 | 27370 | 26048 |
| Tumor2 | 1471 | 839 | 714 | 27764 | 26540 | 25284 |
| Tumor3 | 530 | 339 | 217 | 11068 | 10398 | 9950 |

B) Cosine similarity with WGS (fresh-frozen)

| | Mutect2 | Strelka | Consensus |
|---|---|---|---|
| Tumor1 | 0.96 | 0.94 | 0.92 |
| Tumor2 | 0.95 | 1.00 | 0.99 |
| Tumor3 | 0.96 | 0.84 | 0.83 |

C) Number mutations (FFPE)

| | Mutect2 | Strelka | Consensus |
|---|---|---|---|
| Tumor1 | 383 | 811 | 104 |
| Tumor2 | 47 | 420 | 27 |
| Tumor3 | 90 | 838 | 45 |

D) Cosine similarity with WGS (FFPE)

| | Mutect2 | Strelka | Consensus |
|---|---|---|---|
| Tumor1 | 0.89 | 0.83 | 0.76 |
| Tumor2 | 0.93 | 0.81 | 0.89 |
| Tumor3 | 0.96 | 0.81 | 0.88 |

Supplementary Table 3-Comparison of Mutect2 and Strelka mutation calling pipelines
The tables A and C summarize the number of mutations detected by Mutect2 and Strelka, as well as the overlap/consensus between the two mutation callers, for the three fresh-frozen (mutREAD and WGS) and FFPE tumor samples, respectively.
The cosine similarity of mutREAD-derived and WGS-derived mutational signatures is summarized in tables B and D for the fresh-frozen and FFPE samples, respectively.
For each mutation caller and the consensus set, the mutational signatures were calculated from respective mutREAD-derived and WGS-derived mutation set and compared against each other using cosine similarity.

A) 10x
SWGS

| | | | | | Percent | Base pairs | |
|---|---|---|---|---|---|---|---|
| | | Number of | | Base pairs | of retained | in 10x loci | |
| | | reads after | Properly | covered | reads | shared in | Number |
| Patient | Sample | outer barcode | paired | with at | contributing | tumour/ | of |
| ID | type | demultiplexing | reads | least 10x | to 10x loci | blood pair | mutations |
| 1 | FF | 215,680,416 | 206,317,606 | 2,432,340,493 | 90.21 | 99,329,518 | 42 |
| | Blood | 180,158,855 | 175,855,924 | 2,248,030,642 | 88.49 | — | — |
| 2 | FF | 329,742,782 | 321,605,192 | 2,646,459,519 | 88.55 | 77,553,550 | 21 |
| | Blood | 217,304,771 | 210,403,566 | 2,444,354,485 | 88.51 | — | — |
| 3 | FF | 139,225,587 | 134,967,944 | 1,914,933,524 | 89.19 | 94,152,562 | 83 |
| | Blood | 233,793,837 | 228,289,614 | 2,478,664,018 | 88.27 | — | — |

B) WES

| | | Number of | | | | | |
|---|---|---|---|---|---|---|---|
| | | reads after | | | Percent | Base pairs | |
| | | outer barcode | | Base pairs | of retained | in 10x loci | |
| | | demultiplexing | Properly | covered | reads | shared in | Number |
| Patient | Sample | & PCR clone | paired | with at | contributing | tumour/ | of |
| ID | type | removal | reads | least 10x | to 10x loci | blood pair | mutations |
| 1 | FF | 149,007,546 | 146,883,752 | 228,030,295 | 92.41 | 396,961,129 | 325 |
| | Blood | 72,706,935 | 69,490,692 | 165,989,267 | 89.08 | — | — |
| 2 | FF | 44,956,340 | 44,224,012 | 119,724,582 | 94.68 | 902,341,754 | 142 |
| | Blood | 64,528,333 | 63,067,844 | 145,330,607 | 92.69 | — | — |
| 3 | FF | 74,142,650 | 72,864,976 | 156,263,110 | 82.16 | 251,229,586 | 46 |
| | Blood | 84,750,728 | 79,661,310 | 187,169,707 | 92.76 | — | — |

c) mutREAD

| | | Number of | | | | | |
|---|---|---|---|---|---|---|---|
| | | reads after | | | | | |
| | | outer barcode | | | | | |
| | | demultiplexing, | | | Percent | Base pairs | |
| | | PCR clone | | Base pairs | of retained | in 10x loci | |
| | | removal & | Properly | covered | reads | shared in | Number |
| Patient | Sample | mutREAD | paired | with at | contributing | tumour/ | of |
| ID | type | filtering | reads | least 10x | to 10x loci | blood pair | mutations |
| 1 | FF | 150,115,473 | 142,432,706 | 175,049,803 | 96.54 | 166,936,824 | 1,050 |
| | Blood | 145,505,593 | 137,261,472 | 186,266,055 | 96.26 | — | — |
| 2 | FF | 196,879,399 | 195,040,496 | 195,958,931 | 96.63 | 187,858,494 | 1,471 |
| | Blood | 219,749,023 | 217,691,576 | 193,634,665 | 96.61 | — | — |
| 3 | FF | 216,676,031 | 214,418,984 | 198,984,001 | 96.57 | 170,614,310 | 530 |
| | Blood | 185,303,214 | 175,401,460 | 190,613,393 | 96.49 | — | — |

Supplementary Table 4-Quality metrics for 10x sWGS, WES and mutREAD libraries derived from tumor and blood samples of three patients.
The table summarizes quality metrics of the libraries generated by (A) 10x sWGS, (B) WES, and (C) mutREAD for tumor and blood samples (column 2) of the same three patients (column 1).
Column 3 and 4 gives the number of reads and properly paired read pairs used for mutation calling, respectively.
The number of base pairs covered with at least 10x, the percentage of reads contributing to these loci and the overlap in these loci between tumor and normal samples is listed in column 5-7.

Supplementary TABLE 5
Tumor cellularity of FFPE samples estimated by pathology
A) Tumor biopsies

| Patient | Pathologist 1 | Pathologist 2 | Pathologist 3 |
|---|---|---|---|
| 1 | 70% | 60% | 45% |
| 2 | 50% | 55% | 30% |
| 3 | 30% | 35% | 15-20% |

B) Tumor resections

| Patient | Pathologist 1 | Pathologist 2 | Pathologist 3 |
|---|---|---|---|
| 1 | 60% | 60% | 15% |
| 2 | 60% | 70% | 20% |
| 3 | 30% | 60% | 15% |
| 4 | N/A | N/A | N/A |
| 5 | 30% | 20% | 10-15% |
| 6 | 70% | 40% | 20% |
| 7 | 20% | 45% | 20-25% |
| 8 | 25% | 45% | 50% |
| 9 | 50% | 50% | 20-25% |

A) Estimated percent of tumor content for the three biopsy samples estimated by pathologist review of diagnostic slides.
B) Estimated percent of tumor content for the nine tumor resection samples estimated by pathologist review of diagnostic slides.

Supplementary TABLE 6
Comparative cost evaluation for library preparation per sample

| A) | | | | | |
|---|---|---|---|---|---|
| mutREAD | | | | | |
| No | Reagents | Size | Cost (£) | Use per sample | Per sample (£) |
| 1 | Adapter and Primers | 400 ul | — | 9.5 ul | 2 |
| 2 | T4 ligase | 20,000 Units | 62.58 | 400 units | 1.25 |
| 3 | Apol-HF | 1000 Units | 49.6 | 50 units | 2.48 |
| 4 | Pstl-HF | 10,000 Units | 41.6 | 50 units | 0.2 |
| 5 | 10 mM ATP | 1000 ul | 24 | 4 ul | 0.1 |
| 6 | Ampure XP beads | 5000 ul | 195.26 | 37.5 ul | 1.5 |
| 7 | 10 mM dNTPs | 800 ul | 49.6 | 2 ul | 0.1 |
| 8 | Phusion High fidelity polymerase | 100 Units | 61.6 | 1 unit | 0.6 |
| | | | | Total: | £8.23 |
| B) WES | | | | | |
| SI no | Reagents | Size | Cost (£) | Per sample (£) | |
| 1 | DNA library preparation and enrichment kit | 16 | 3,589 | 199 | |
| | | | Total: | £199 | |
| C) 10× | | | | | |
| sWGS | | | | | |
| SI no | Reagents | Size | Cost (£) | Per sample (£) | |
| 1 | Thruplex library preparation and enrichment kit | 96 | 3,818.59 | 39.7 | |
| 2 | Sonication | 96 | 352 | 3.7 | |
| | | Total: | £43 | | |

A) Estimated cost of individual elements used for library preparation using the mutREAD protocol. The cost is estimated using reagents provided by New England Biolab, Ipswich, MA 01938 USA
B) Cost estimate of enrichment-based whole exome sequencing provided by Agilent, Santa Clara, CA 95051 USA. The cost does not include AMPure XP and Streptavidin beads required for the selection of target sequences.
C) Cost estimate of the whole genome library preparation method provided by Takara Bio, Kusatsu, Shiga 525-0058, Japan. The cost does not include AMPure XP and Streptavidin beads required for the selection of target sequences.
Cost of sequencing: Assuming 200× coverage for mutREAD, the per-sample cost is around £150. The costs for WES would be similar as both methods sample a comparable proportion of the genome. 10× sWGS would cost £300-£700 depending on the chosen sequencing platform.

SUPPLEMENTARY TABLE 7
List of restriction enzymes tested in the computa-
tional simulation and their restriction site
sequences
The table lists the enzymes selected as described
in the Methods section and their restriction sites
(5′→3′), with the cutting position indicated by *,
highlighting the different possible overhangs.
Ambiguous codes R and Y translate to A/G or C/T,
respectively, and indicate that either base at
this position is accepted by the enzyme.

| Restriction enzyme | Restriction site sequence |
|---|---|
| AflII | C*TTAAG |
| ApoI | R*AATTY |
| BmtI | GCTAG*C |
| BsrGI | T*GTACA |
| BssSI | C*ACGAG |
| HindIII | A*AGCTT |
| KpnI | GGTAC*C |
| MfeI | C*AATTG |
| MseI | T*TAA |
| MspI | C*CGG |
| NcoI | C*CATGG |
| NdeI | CA*TATG |
| NsiI | ATGCA*T |
| NspI | RCATG*Y |
| PacI | TTAAT*TAA |
| PstI | CTGCA*G |
| SbfI | CCTGCA*GG |
| SpeI | A*CTAGT |
| SphI | GCATG*C |

### EXAMPLE 2

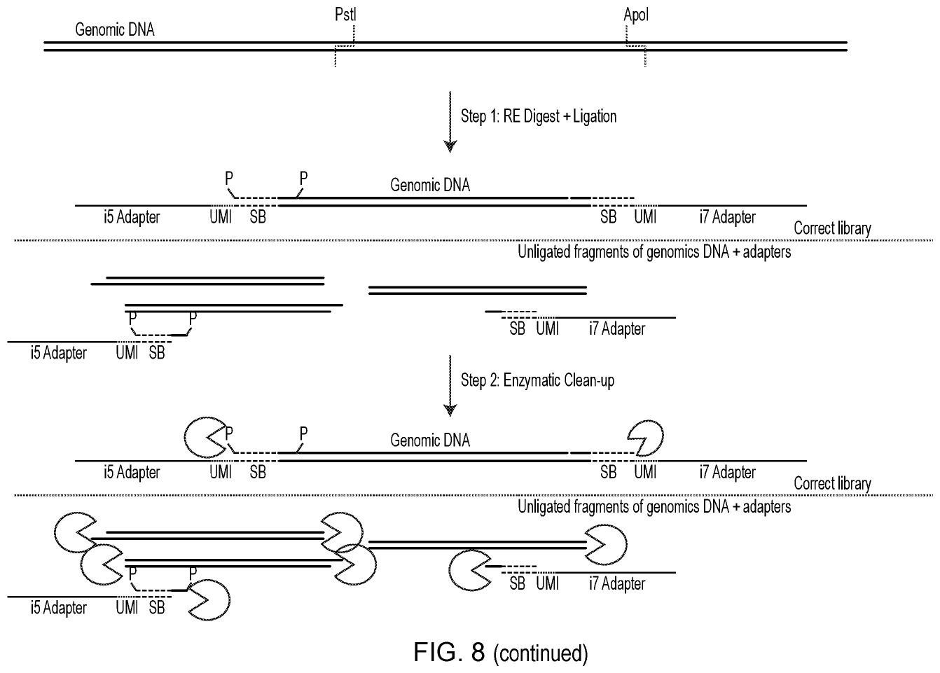

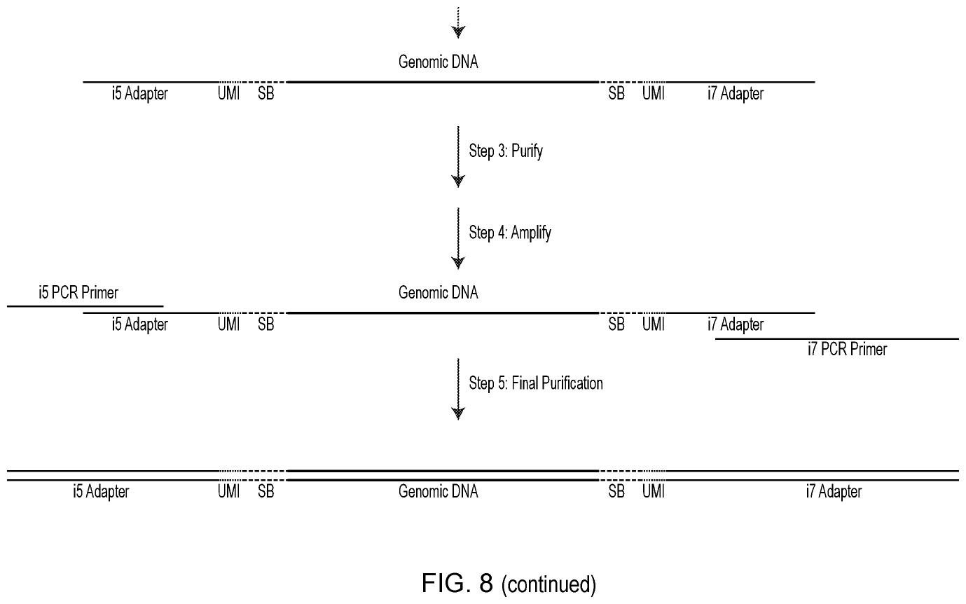

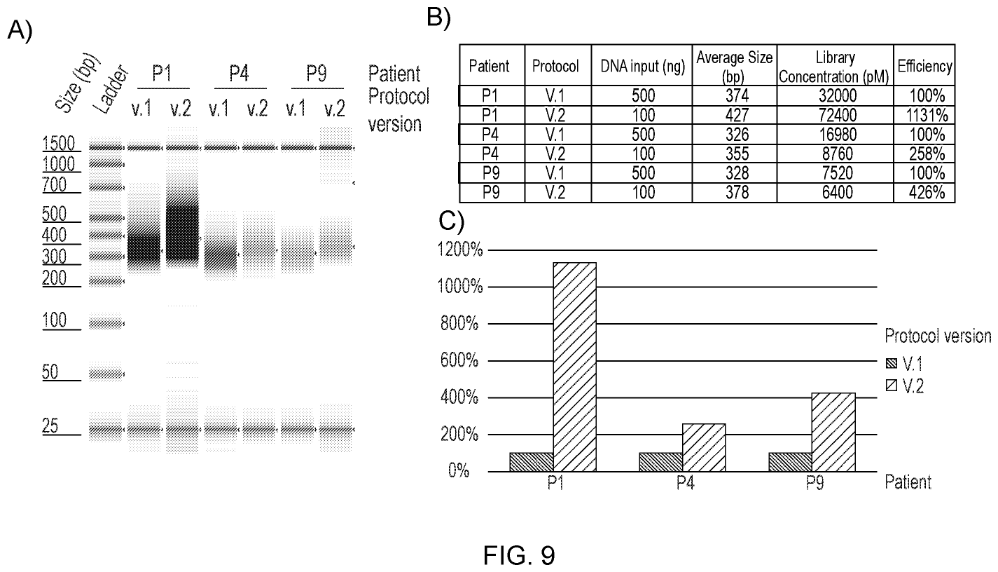

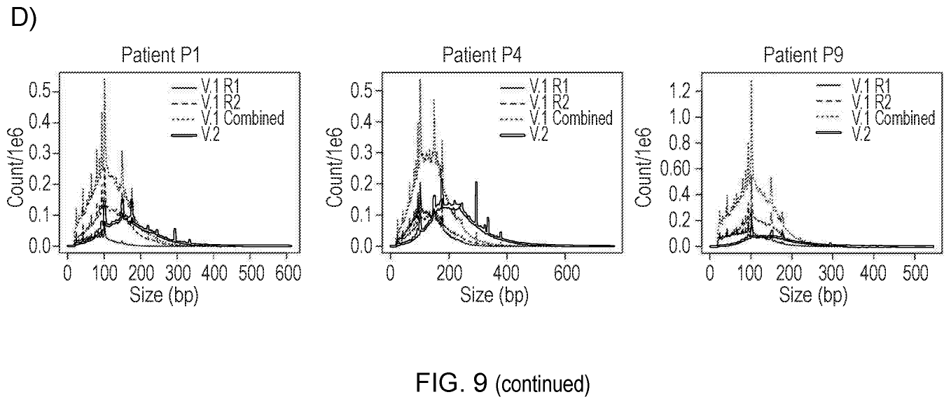

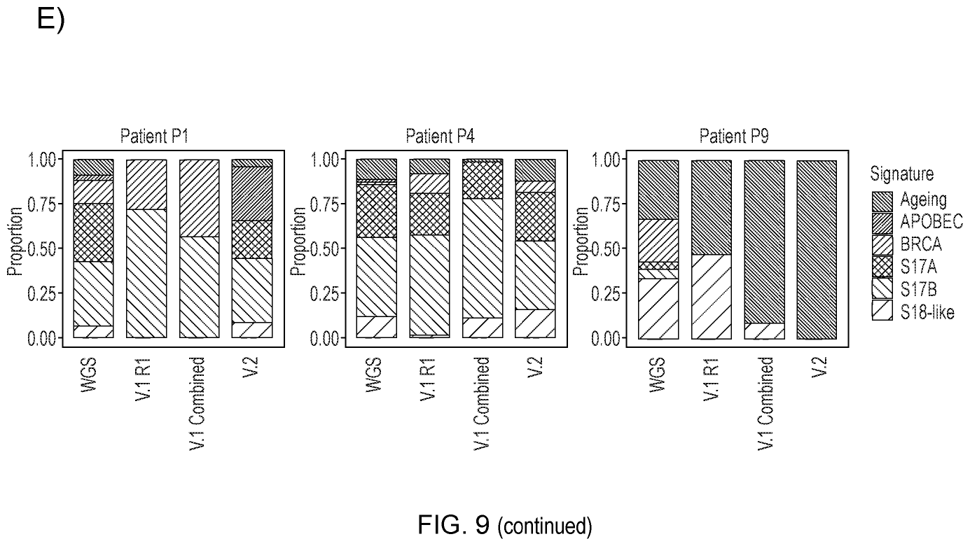

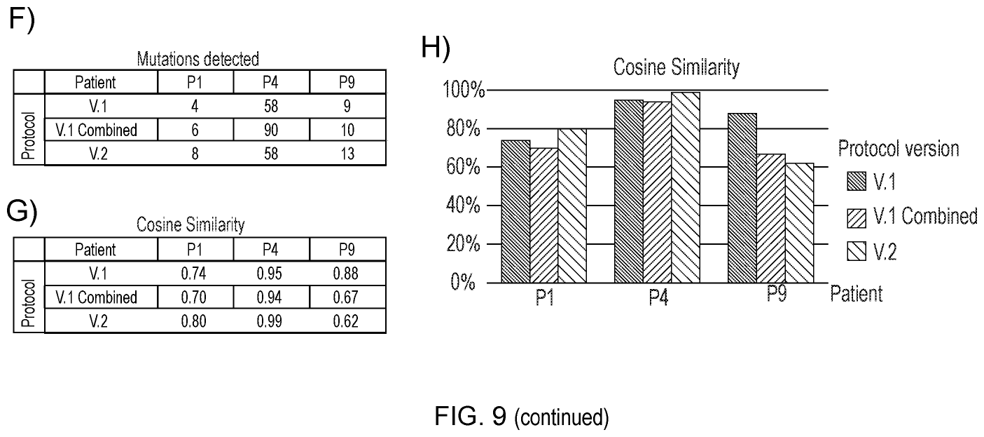

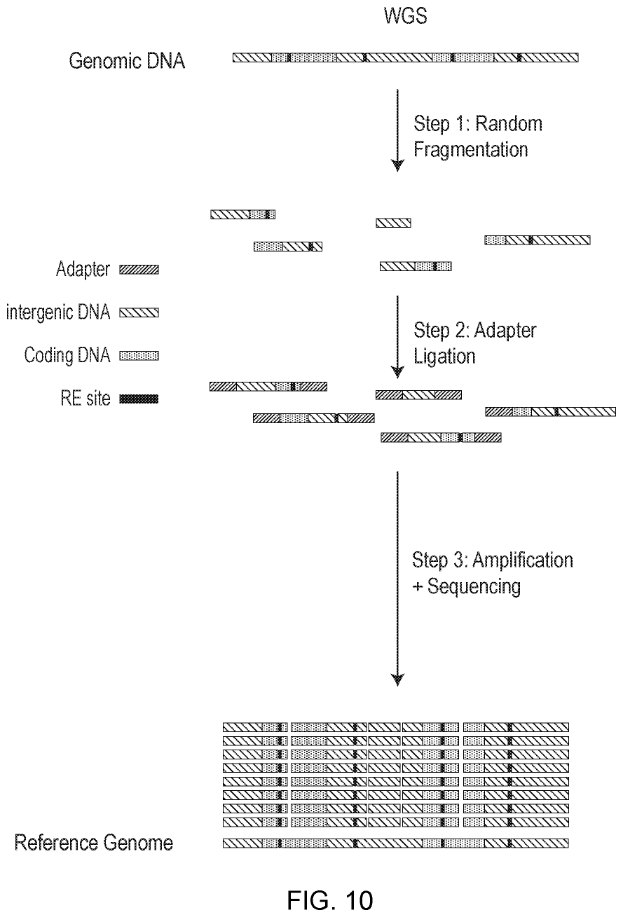

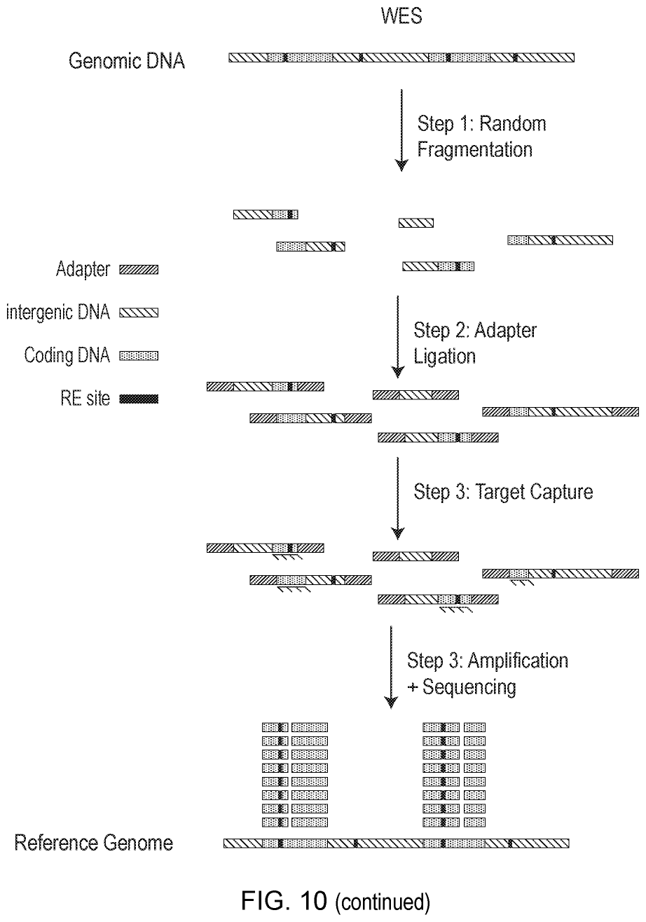

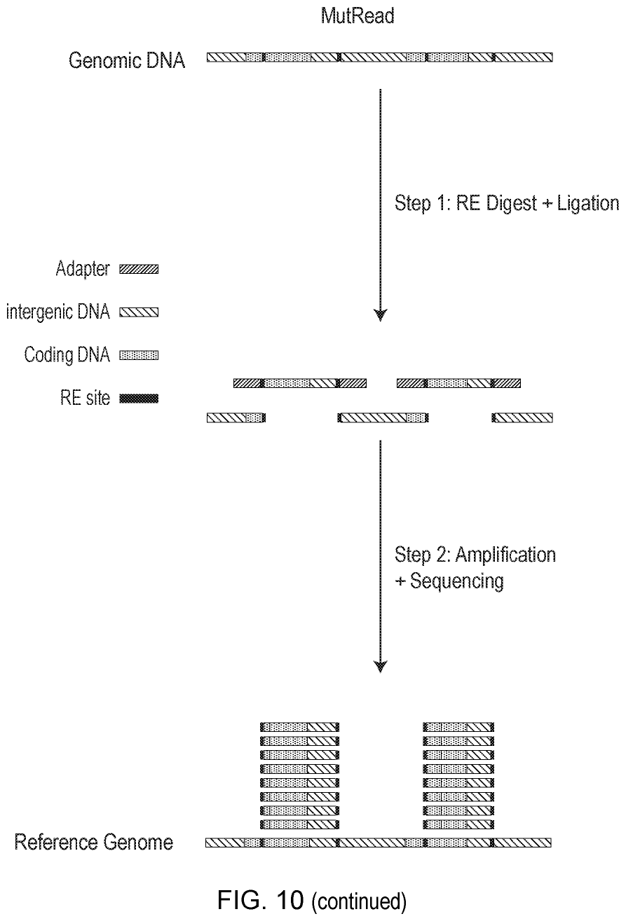

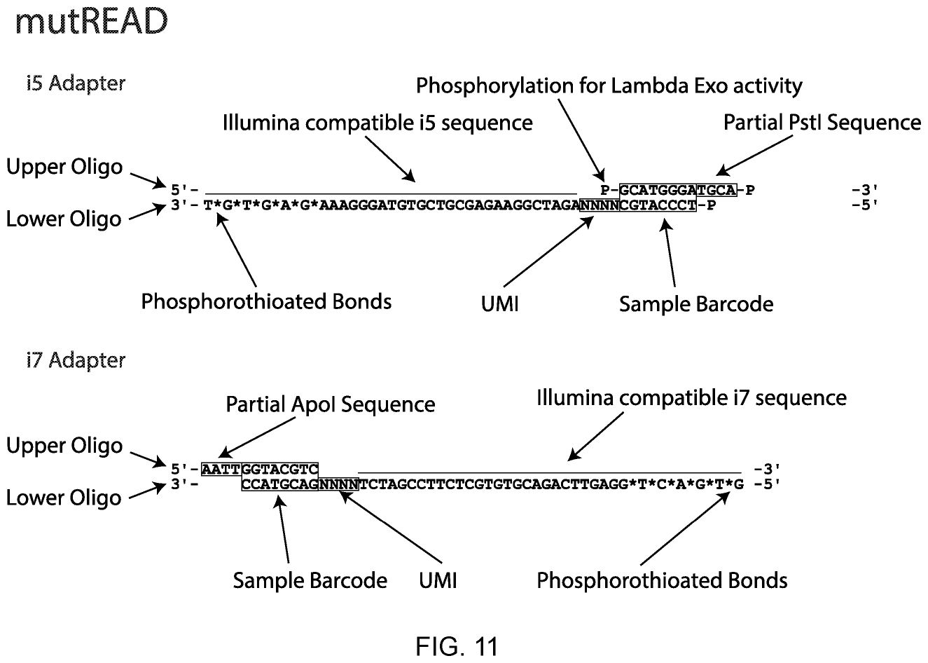

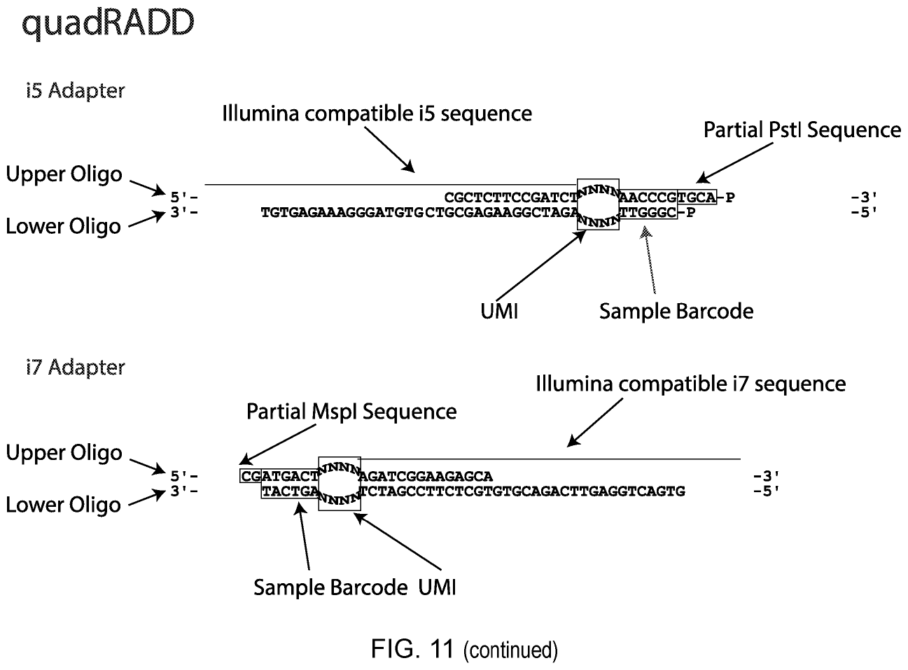

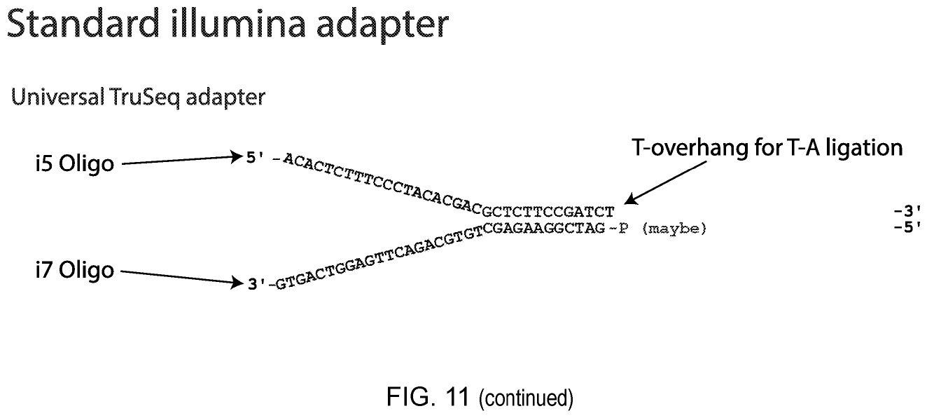

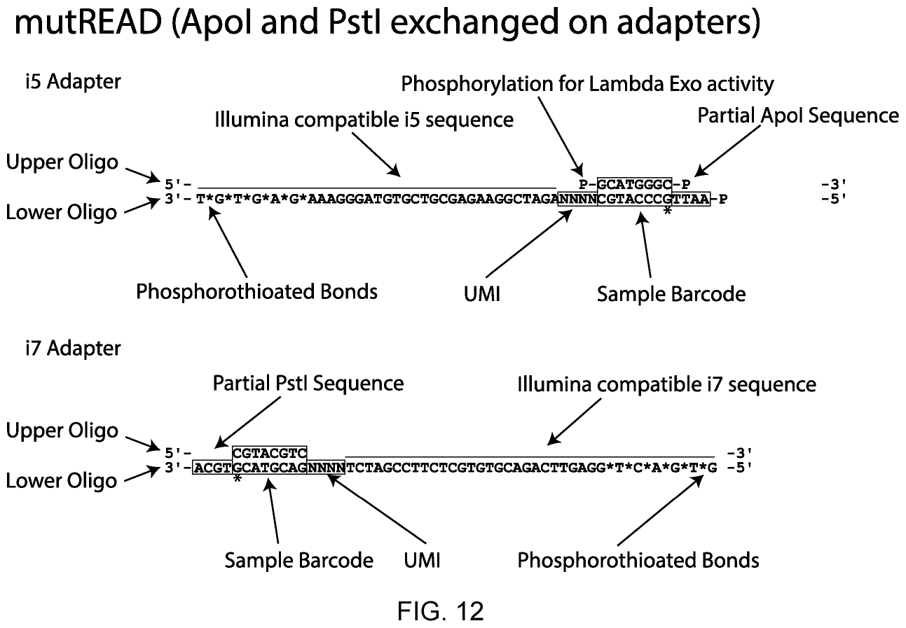

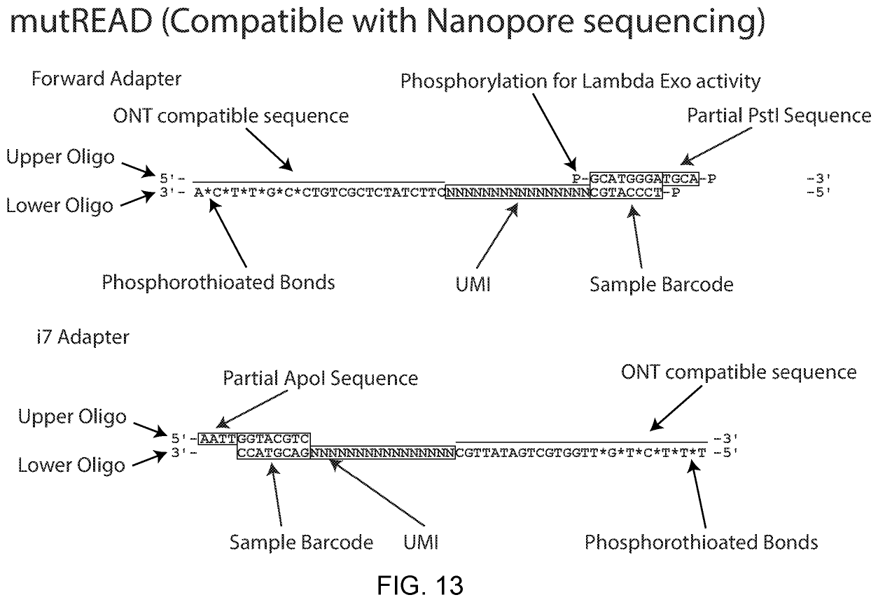

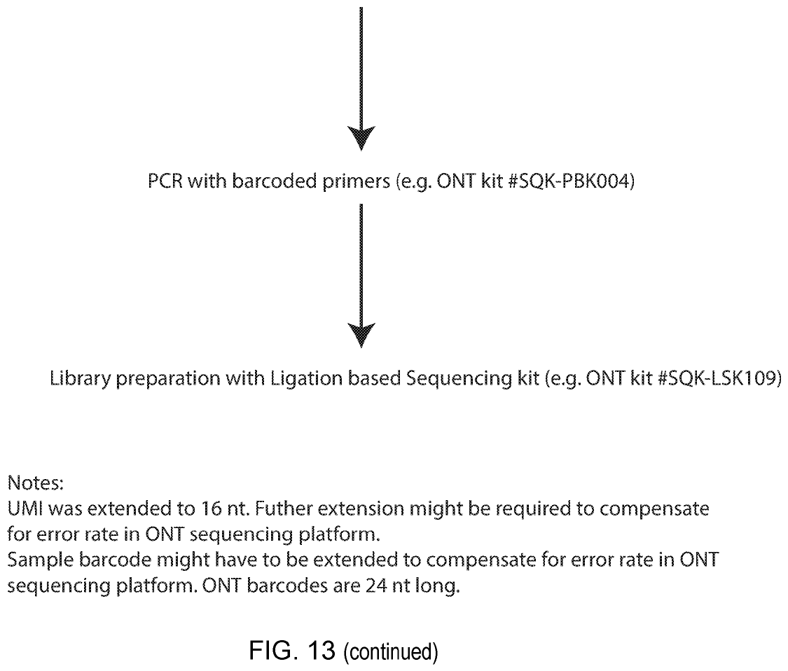

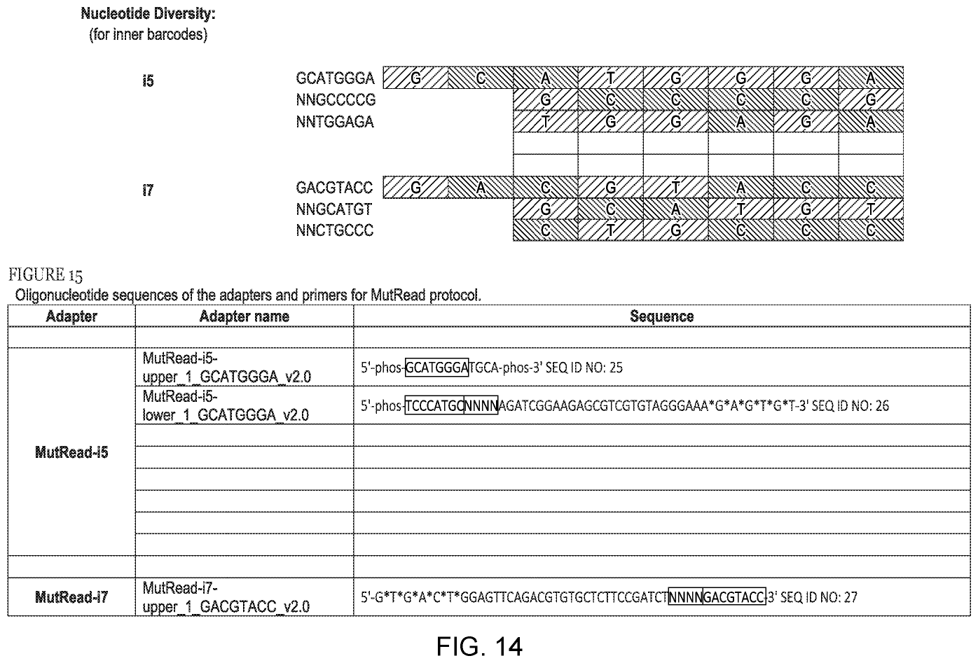

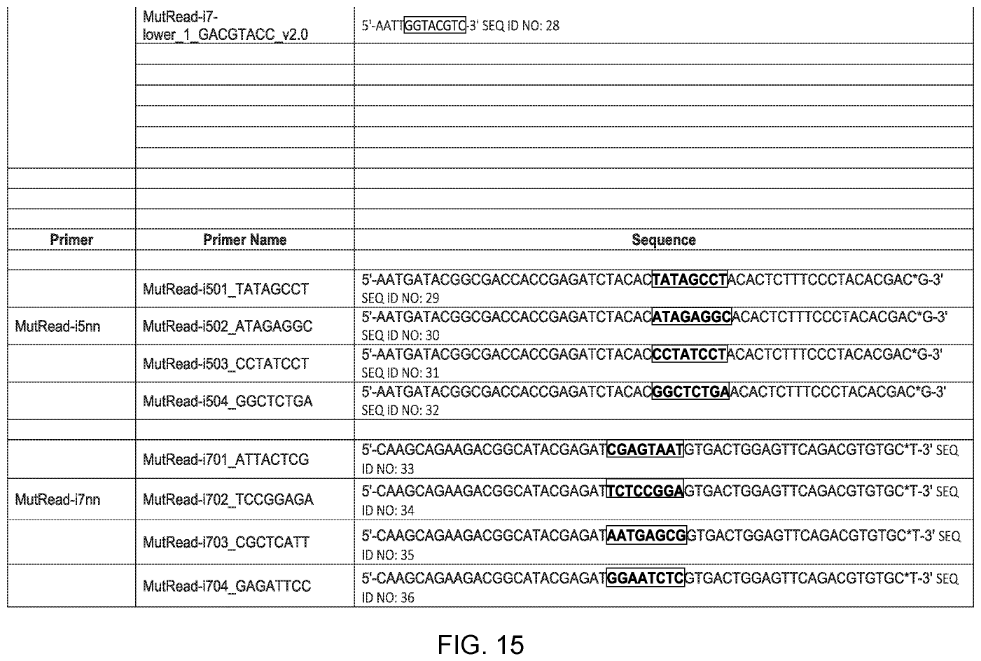

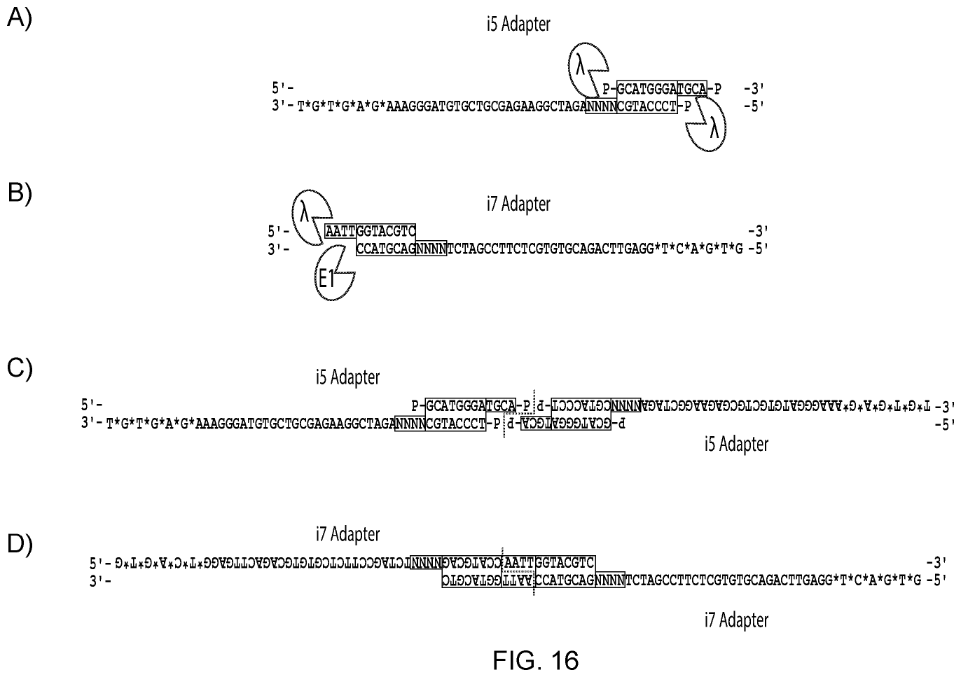

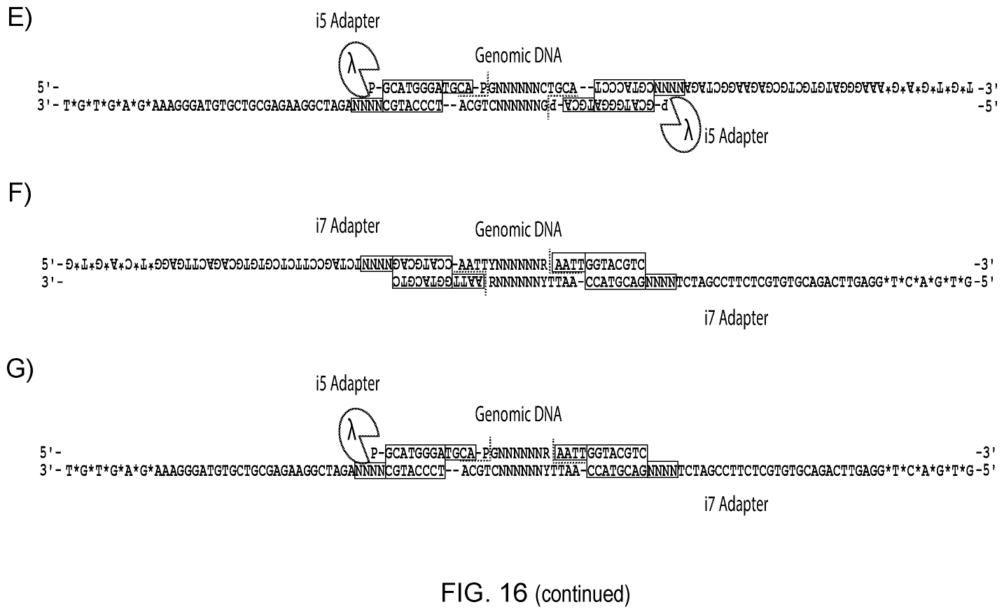

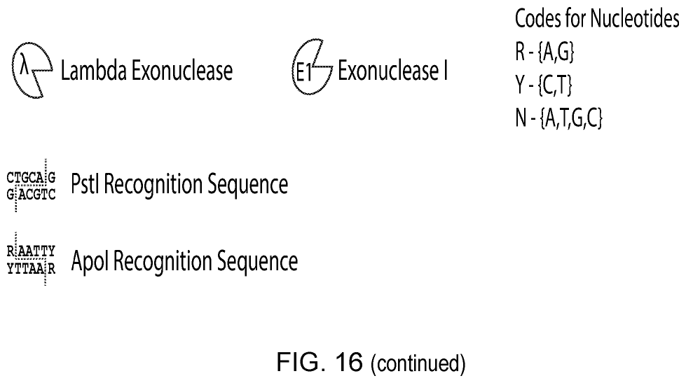

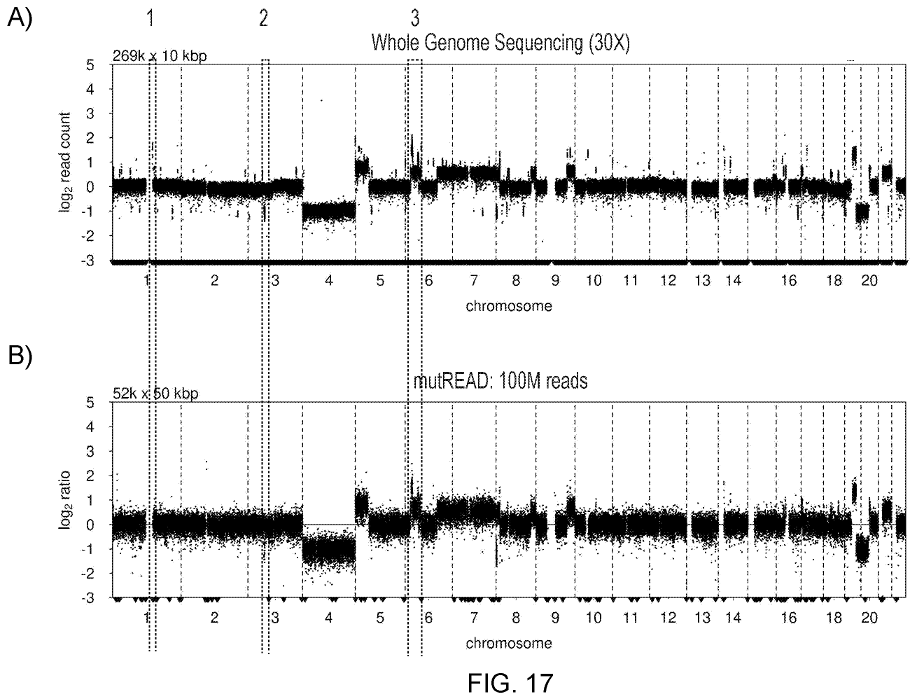

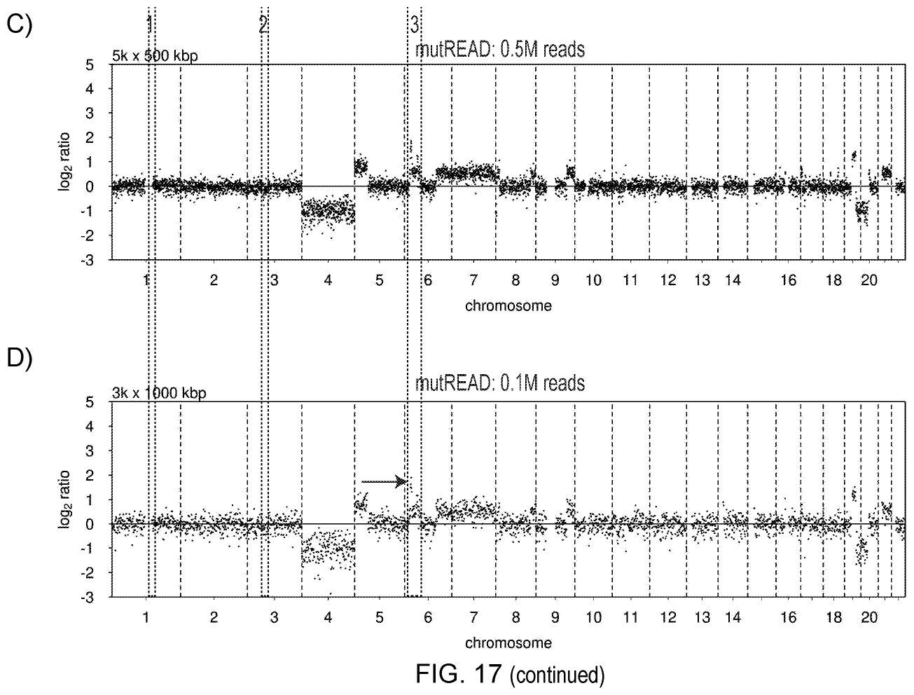

A sequencing library containing a subset of the genome is generated by digesting the samples with two restriction enzymes (FIG. 8). Our protocol allows for sequencing of fragments with a specific size range containing restriction enzyme sites. The computational analysis allows for the accurate quantification of the exposure for pre-defined mutational signatures (FIG. 9).

In contrast to known WGS, in the invention only parts of the genome are sequenced. The method relies on the fact that restriction enzyme target sites are randomly distributed, yet fixed within the genome. As a result, when the same combination of enzymes is applied to different samples, identical fragments are produced. Such selection of sequencing regions removed inherited bias introduced by other known methods proposed as alternatives to WGS (FIG. 10):
low coverage (<10×) WGS results in non-reproducible mutation calling and has a limited (3 to 5-fold reduction) financial advantage;
exome sequencing is inherently biased toward regions that undergo evolutionary selection/pressure and does not allow for efficient signature calling

In order to achieve the desired comparability of the invention with known WGS we:
computationally selected an optimal combination of restriction enzymes that capture a sufficient number of mutations and cover less than 10% of the human genome;
introduced steps that remove FFPE specific artefacts;
introduced steps that maximise DNA conversion into sequencing libraries;
removed the requirement for highly specialised equipment during the library preparation procedure (FIG. 10).

TABLE

| see FIG. 10 | WGS | WES | mutREAD |
|---|---|---|---|
| Genomic regions | Unbiased | Only Coding | Unbiased |
| covered | representation of | | representation of |
| | genome | | genome |
| Part of genome | ~90% | ~2% | 0.1 to 10% |
| sequenced (%) | | | |
| Cost in | 100% | 20-40% | ~10% |
| comparison to | | | |
| WGS | | | |

Thus the invention is capable of capturing mutational signature at a fraction of WGS cost (estimated i-fold reduction in cost) with comparable specificity and sensitivity. Sequencing adapters that align to the restriction enzyme-specific overhangs allow for the specific selection of a reproducible set of random sequencing fragments. This reduces off-target fragments and redirects the sequencing power to the fragments of interest. This aspect is especially useful for low-quality FFPE samples that, by their nature, are highly fragmented.

The invention allows for multiplexing of a large number of samples due to a double barcode system. With the development of new sequencing platforms, high multiplexing capabilities will make the efficient use of the increasing sequencing capabilities feasible. The costs of the method will continue to scale down with sequencing costs. Our method can be performed manually on batches of samples (up to 96 in standard setting) requiring little hands-on time compared to known WGS, known shallow WGS and known exome sequencing or can be automated using robotic library preparation systems.

We present data for WGS (prior art) and mutREAD (invention) comparison. In order to confirm performance improvement of the method of the invention, we constructed and sequenced mutREAD libraries from three tumour samples using a method virtually the same as the known method described by (Franchini et al ibid.) (“v.1”) and our improved method of the invention (“v.2”). We used 500 ng of input DNA for v.1 and 100 ng for v.2. The v.2 protocol (the invention) consistently produced higher amounts of libraries using similar amplification rates (FIG. 9A-C). We further observed improvement in the median size of libraries that were also visible in the distribution of sequenced fragments (FIG. 9A, B, D). V.2 of the protocol also outperformed Franchini et al “v.1” in its ability to call mutational signatures (FIG. 9 E) showing an excellent similarity with WGS data (FIG. 9 F-H).

### EXAMPLE 3: ENZYMATIC CLEAN-UP

This example shows the benefits of enzymatic clean-up step such as nuclease digestion. We refer to FIG. 16: Rationale behind enzymatic clean-up of the samples. Removal of partially ligated (only one adapter) on unligated genomic DNA is not shown. It will be removed by Lambda Exonuclease and Exonuclease I.
A) Removal of unligated first adapters (e.g. i5 adapters) by Lambda Exonuclease;
B) Removal of unligated second adapters (e.g. i7 adapters) by Lambda Exonuclease and Exonuclease I (ssDNA produced by Lambda Exonuclease);
C) Phosphate groups at 3′ ends of first adapters prevent adapter dimers and unligated first adapters can be removed as in (A);
D) Lack of phosphate groups at 5′ ends of second adapters prevent adapter dimers and unligated second adapters can be removed as in (B);
E) Ligation of first adapters to genomic DNA fragments having two PstI compatible ends results in covalent bond only between lower oligo of first adapter and genomic DNA (upper oligo binding is prevented by phosphate group at 3′-end). Double stranded DNA is removed by Lambda Exonuclease;
F) Ligation of second adapters to genomic DNA fragments having two ApoI compatible ends results in covalent bond only between lower oligo of adapter and genomic DNA (upper oligo binding is prevented by absence of phosphate group at 5′-end). Double stranded DNA is cannot be removed by neither Lambda Exonuclease nor Exonuclease I. However, the fragments will not be amplified during subsequent PCR due to absence of 3′ extensions complementary to PCR primers;
G) Correctly ligated genomics DNA fragments with first and second adapters at opposite ends are protected from degradation by phosphorothioated bonds. The upper oligos of first adapter will be removed by Lambda Exonuclease. Only one strand of DNA can be amplified in subsequence PCR.

### EXAMPLE 4: APPLICATION TO COPY NUMBER ALTERATION

In addition to use of the invention (‘MutREAD’) to accurately estimate mutational signatures, we also tested whether copy number alteration (CNA) can be identified using mutREAD data. The analysis was performed using Flo-1 cancer cell line that was subjected to either Whole Genome Sequencing (WGS) at 30× coverage or mutREAD at 100M reads coverage. We also developed custom computational pipeline that considers data structure produced by mutREAD and corrects artefacts that may be introduced by the method. In the direct comparison of WGS and mutREAD at high depth (100M) we observed excellent concordance between the method with mutREAD effectively capturing chromosome arm changes and focal CNA (<5 Mbp) (FIGS. 17 A and B, regions 2 and 3). In a small number of instances mutREAD data did not capture focal CNA (FIGS. 17 A and B, region 1). We further tested whether CNA can be identified in the mutREAD data when down-sampled to lower coverage:
0.5M reads—equivalent of 0.05× genome-wide and 0.5-1× in the mutREAD target regions
0.1M reads—equivalent of 0.01× genome-wide and 0.1-0.2× in the mutREAD target regions

In general, the pattern of large (chromosome arm) changes was recapitulated by low coverage mutREAD (FIGS. 17C and D). Due to the sparsity of data most of the focal CNA could not be identified in the data, although some highly amplified focal CNA were visible (region 3, arrow, FIGS. 17C and D).

Taken together, this data shows that computational analysis of mutREAD data allows for accurate identification of CNA in mutREAD data that is highly concordant with the gold-standard, WGS results.

### REFERENCES

1. Alexandrov, L. B. et al. Signatures of mutational processes in human cancer. Nature 500, 415-21 (2013).
2. Alexandrov, L. B. et al. The repertoire of mutational signatures in human cancer. Nature 578, 94-101 (2020).
3. Nik-Zainal, S. et al. Mutational processes molding the genomes of 21 breast cancers. Cell 149, 979-993 (2012).
4. Nik-Zainal, S. et al. Landscape of somatic mutations in 560 breast cancer whole-genome sequences. Nature 534, 1-20 (2016).
5. Northcott, P. A. et al. The whole-genome landscape of medulloblastoma subtypes. Nature 547, 311-317 (2017).
6. Kucab, J. E. et al. A Compendium of Mutational Signatures of Environmental Agents. Cell 177, 1-16 (2019).
7. Pich, O. et al. Somatic and Germline Mutation Periodicity Follow the Orientation of the DNA Minor Groove around Nucleosomes. Cell 175, 1074-1087.e18 (2018).
8. Petljak, M. et al. Characterizing Mutational Signatures in Human Cancer Cell Lines Reveals Episodic APOBEC Mutagenesis. Cell 176, 1282-1294.e20 (2019).
9. Alexandrov, L. B. et al. Mutational signatures associated with tobacco smoking in human cancer. Science (80-.). 354, 618-622 (2016).
10. Lee-Six, H. et al. The landscape of somatic mutation in normal colorectal epithelial cells. Nature 574, 532-537 (2019).
11. Martincorena, I. et al. Somatic mutant clones colonize the human esophagus with age. Science (80-.). 362, 911-917 (2018).
12. Brunner, S. F. et al. Somatic mutations and clonal dynamics in healthy and cirrhotic human liver. Nature 574, 538-542 (2019).
13. Secrier, M. et al. Mutational signatures in esophageal adenocarcinoma define etiologically distinct subgroups with therapeutic relevance. Nat. Genet. 48, 1131-1141 (2016).
14. Davies, H. et al. HRDetect is a predictor of BRCA1 and BRCA2 deficiency based on mutational signatures. Nat. Med. 23, 517-525 (2017).
15. Staaf, J. et al. Whole-genome sequencing of triple-negative breast cancers in a population-based clinical study. Nat. Med. 25, 1526-1533 (2019).
16. Momen, S. et al. Dramatic response of metastatic cutaneous angiosarcoma to an immune checkpoint inhibitor in a patient with xeroderma pigmentosum: whole-genome sequencing aids treatment decision in end-stage disease. Cold Spring Harb. Mol. case Stud. 5, 1-11 (2019).
17. Polak, P. et al. A mutational signature reveals alterations underlying deficient homologous recombination repair in breast cancer. Nat. Genet. 49, 1476-1486 (2017).
18. Connor, A. A. et al. Association of Distinct Mutational Signatures With Correlates of Increased Immune Activity in Pancreatic Ductal Adenocarcinoma. JAMA Oncol. 3, 774 (2017).
19. Zehir, A. et al. Mutational landscape of metastatic cancer revealed from prospective clinical sequencing of 10,000 patients. Nat. Med. (2017). doi:10.1038/nm.4333
20. Angus, L. et al. The genomic landscape of metastatic breast cancer highlights changes in mutation and signature frequencies. Nat. Genet. 51, 1450-1458 (2019).
21. Franchini, P., Monné Parera, D., Kautt, A. F. & Meyer, A. quaddRAD: a new high-multiplexing and PCR duplicate removal ddRAD protocol produces novel evolutionary insights in a nonradiating cichlid lineage. Mol. Ecol. 26, 2783-2795 (2017).
22. Perry, E. B. et al. Tumor diversity and evolution revealed through RADseq. Oncotarget 8, 41792-41805 (2017).
23. Cibulskis, K. et al. Sensitive detection of somatic point mutations in impure and heterogeneous cancer samples. Nat. Biotechnol. 31, 213-219 (2013).
24. Saunders, C. T. et al. Strelka: accurate somatic small-variant calling from sequenced tumor-normal sample pairs. Bioinformatics 28, 1811-1817 (2012).
25. Dulak, A. M. et al. Exome and whole-genome sequencing of esophageal adenocarcinoma identifies recurrent driver events and mutational complexity. Nat. Genet. 45, 478-86 (2013).
26. Gulhan, D. C., Lee, J. J.-K., Melloni, G. E. M., Cortes-Ciriano, I. & Park, P. J. Detecting the mutational signature of homologous recombination deficiency in clinical samples. Nat. Genet. 51, 912-919 (2019).
27. Zheng, C. et al. Determination of genomic copy number alteration emphasizing a restriction site-based strategy of genome re-sequencing. Bioinformatics 29, 2813-2821 (2013).
28. Mora-Mirquez, F., Garcia-Olivares, V., Emerson, B. C. & López de Heredia, U. ddradseqtools: a software package for in silico simulation and testing of double-digest RADseq experiments. Mol. Ecol. Resour. 17, 230-246 (2017).
29. Inc, I. Nextera Rapid Capture Enrichment Reference Guide. Illumina Propr. (2015).
30. Illumina. Illumina Adapter Sequences Introduction 3 Sequences for Nextera Kits 3 Sequences for AmpliSeq for Illumina Panels 16 Sequences for TruSight Kits 18 Sequences for TruSeq Kits 24 Process Controls for TruSeq Kits 36 Legacy Kits 42 Revision History 48 Technic. (2019).
31. Rochette, N. C. & Catchen, J. M. Deriving genotypes from RAD-seq short-read data using Stacks. Nat. Protoc. 12, 2640-2659 (2017).
32. Li, H. & Durbin, R. Making the Leap: Maq to BWA. Mass Genomics 25, 1754-1760 (2009).
33. Li, H. et al. The Sequence Alignment/Map format and SAMtools. Bioinformatics 25, 2078-2079 (2009).
34. Http://broadinstitute.github.io/picard/. “Picard Toolkit.” 2019. Broad Institute, GitHub Repository.
35. Gehring, J. S., Fischer, B., Lawrence, M. & Huber, W. SomaticSignatures: Inferring mutational signatures from single-nucleotide variants. Bioinformatics 31, 3673-3675 (2015).

| IUPAC nucleotide code | Base |
|---|---|
| A | Adenine |
| C | Cytosine |
| G | Guanine |
| T (or U) | Thymine (or Uracil) |
| R | A or G |
| Y | C or T |
| S | G or C |
| W | A or T |
| K | G or T |
| M | A or C |
| B | C or G or T |
| D | A or G or T |
| H | A or C or T |
| V | A or C or G |
| N | any base |

TABLE OF SEQUENCES
SEQ ID NO: 1 (Illumina i7 flow cell binding site for
immobilisation)
ATCTCGTATGCCGTCTTCTGCTTG
SEQ ID NO: 2 (Illumina i5 flow cell binding site for
immobilisation)
AATGATACGGCGACCACCGAGATCTACAC
SEQ ID NO: 3 (binding site for at least one oligonucleotide
primer - Illumina i7 adapter/primer) (33 nt)
GATCGGAAGAGCACACGTCTGAACTCCAGTCAC
SEQ ID NO: 4 (binding site for at least one oligonucleotide
primer - Illumina i5 adapter/primer) (33 nt)
ACACTCTTTCCCTACACGACGCTCTTCCGATCT
SEQ ID NO: 5 (binding site for at least one oligonucleotide
primer - Illumina i7 adapter/primer) (34 nt including additional
5′ T)
AGATCGGAAGAGCACACGTCTGAACTCCAGTCAC
SEQ ID NO: 6 (binding site for at least one oligonucleotide
primer - Illumina i7 adapter/primer) (33 nt) (reverse complement
of SEQ ID NO: 3)
5′-GTGACTGGAGTTCAGACGTGTGCTCTTCCGATC-3′
SEQ ID NO: 7 (binding site for at least one oligonucleotide
primer - Illumina i5 adapter/primer) (33 nt) (reverse complement
of SEQ ID NO: 4)
5′-AGATCGGAAGAGCGTCGTGTAGGGAAAGAGTGT-3′
SEQ ID NO: 8 (binding site for at least one oligonucleotide
primer - Illumina i7 adapter/primer) (34 nt including additional
5′ T) (reverse complement of SEQ ID NO: 5)
5′-GTGACTGGAGTTCAGACGTGTGCTCTTCCGATCT-3′
SEQ ID NO: 9 (ONT forward/left end (i5) adapter/primer i.e.
binding site for at least one oligonucleotide primer - first
adapter)
5′-CTTCTATCTCGCTGTCCGTTCA-3′
SEQ ID NO: 10 (ONT reverse/right end (i5) adapter/primer i.e.
binding site for at least one oligonucleotide primer - second
adapter)
5′-TTTCTGTTGGTGCTGATATTGC-3′

1. A pair of oligonucleotide adapters,
wherein said pair comprises a first oligonucleotide adapter comprising
(a) a top strand comprising 5′—N₈₋₂₄ barcode sequence—N₁₋₅ sequence corresponding to a sticky end left by digestion by a first restriction enzyme—phosphate—3′
wherein at least one of the nucleotide(s) of the N₈₋₂₄ barcode sequence immediately adjacent to the N₁₋₅ sequence corresponding to the sticky end left by digestion by said first restriction enzyme is different to the corresponding nucleotide(s) of the recognition sequence of said first restriction enzyme; and
a bottom strand comprising 5′—phosphate—N₈₋₂₄ barcode sequence complementary to the N₈₋₂₄ barcode sequence of the top strand—N₄₋₂₄ unique molecular identifier (UMI) sequence—binding site for at least one oligonucleotide primer—3′
wherein at least the 6 bases at the 3′ terminal end of the bottom strand are each phosphorothioated;
and a second oligonucleotide adapter comprising
(b) a top strand comprising 5′—N₁₋₅ sequence corresponding to sticky end left by a second restriction enzyme—N₈₋₂₄ barcode sequence—3′
wherein at least one of the nucleotide(s) of the N₈₋₂₄ barcode sequence immediately adjacent to the N₁₋₅ sequence corresponding to the sticky end left by said second restriction enzyme is different to the corresponding nucleotide(s) of the recognition sequence of said second restriction enzyme; and
a bottom strand comprising 5′—binding site for at least one oligonucleotide primer—N₄₋₂₄ unique molecular identifier (UMI) sequence—N₈₋₂₄ barcode sequence complementary to the N₈₋₂₄ barcode sequence of the top strand—3′
wherein at least the 6 bases at the 5′ terminal end of the bottom strand are each phosphorothioated;
or
wherein said pair comprises a first oligonucleotide adapter comprising
(c) a top strand comprising 5′—N₈₋₂₄ barcode sequence—phosphate—3′; and
a bottom strand comprising 5′—phosphate—N₁₋₅ sequence corresponding to sticky end left by digestion by a first restriction enzyme—N₈₋₂₄ barcode sequence complementary to the N₈₋₂₄ barcode sequence of the top strand—N₄₋₂₄ unique molecular identifier (UMI) sequence—binding site for at least one oligonucleotide primer—3′
wherein at least one of the nucleotide(s) of the N₈₋₂₄ barcode sequence immediately adjacent to the N₁₋₅ sequence corresponding to the sticky end left by digestion by said first restriction enzyme is different to the corresponding nucleotide(s) of the recognition sequence of said first restriction enzyme,
wherein at least the 6 bases at the 3′ terminal end of the bottom strand are each phosphorothioated;
and a second oligonucleotide adapter comprising
(d) a top strand comprising 5′—N₈₋₂₄ barcode sequence—3′; and
a bottom strand comprising 5′—binding site for at least one oligonucleotide primer—N₄₋₂₄ unique molecular identifier (UMI) sequence—N₈₋₂₄ barcode sequence complementary to the N₈₋₂₄ barcode sequence of the top strand—N₁₋₅ sequence corresponding to sticky end left by a second restriction enzyme—3′
wherein at least one of the nucleotide(s) of the N₈₋₂₄ barcode sequence immediately adjacent to the N₁₋₅ sequence corresponding to the sticky end left by said second restriction enzyme is different to the corresponding nucleotide(s) of the recognition sequence of said second restriction enzyme,
wherein at least the 6 bases at the 5′ terminal end of the bottom strand are each phosphorothioated.

2. A pair of oligonucleotide adapters according to claim 1 wherein said oligonucleotide top strand of (a) and/or (c) further comprises a phosphate group at its 5′ terminal end.

3. A pair of oligonucleotide adapters according to claim 1, wherein said N₈₋₂₄ barcode sequence is a N₈₋₁₂ barcode sequence.

4. A pair of oligonucleotide adapters according to claim 1, wherein said N₄₋₂₄ unique molecular identifier (UMI) sequence is a N₄₋₁₆ unique molecular identifier (UMI) sequence.

5. A pair of oligonucleotide adapters according to claim 1
wherein said N₈₋₂₄ barcode sequence is a N₈ barcode sequence, and
wherein said N₄₋₂₄ unique molecular identifier (UMI) sequence is a N₄ unique molecular identifier (UMI) sequence.

6. A pair of oligonucleotide adapters according to claim 5 wherein the binding site for at least one oligonucleotide primer of the bottom strand of strand of (a) and/or (c) comprises, or consists of, SEQ ID NO: 7, and wherein the binding site for at least one oligonucleotide primer of the bottom strand of strand of (b) and/or (d) comprises, or consists of, SEQ ID NO: 6 or SEQ ID NO: 8.

7. A pair of oligonucleotide adapters according to claim 1
wherein said N₈₋₂₄ barcode sequence is a N₂₄ barcode sequence, and
wherein said N₄₋₂₄ unique molecular identifier (UMI) sequence is a N₁₆ unique molecular identifier (UMI) sequence.

8. A pair of oligonucleotide adapters according to claim 7 wherein the binding site for at least one oligonucleotide primer of the bottom strand of strand of (a) and/or (c) comprises, or consists of, SEQ ID NO: 9, and wherein the binding site for at least one oligonucleotide primer of the bottom strand of strand of (b) and/or (d) comprises, or consists of, SEQ ID NO: 10.

9. A pair of oligonucleotide adapters according to claim 1, wherein the top strand N₈₋₂₄ barcode sequence and the bottom strand N₈₋₂₄ barcode sequence complementary to the N₈₋₂₄ barcode sequence of the top strand are present as double stranded nucleic acid within the adapter.

10. A pair of oligonucleotide adapters according to claim 1, wherein said first and second restriction enzymes comprise
(i) an enzyme having the recognition site 5′CTGCA/G3′; and
(ii) an enzyme having the recognition site 5′R/AATTY3′

11. A pair of oligonucleotide adapters according to claim 1, wherein said first and second restriction enzymes comprise
(i) PstI; and
(ii) ApoI.

12. A pair of oligonucleotide adapters according to claim 1, wherein the N₁₋₅ sequence of (a) and (d) comprises TGCA and wherein the N₁₋₅ sequence of (b) and (c) comprises AATT.

13. A method of preparing a nucleic acid library from a sample comprising high molecular weight DNA (HMW DNA) comprising the steps
(i) contacting said DNA with a first restriction enzyme and a second restriction enzyme;
(ii) contacting said DNA with a pair of oligonucleotide adapters according to claim 1;
(iii) contacting said DNA with at least one DNA ligase; and
(iv) incubating to allow digestion of the DNA by said first restriction enzyme and second restriction enzyme, annealing of said oligonucleotide adapters to the digested DNA, and ligation of the annealed oligonucleotide adapters to the digested DNA by said at least one DNA ligase.

14. A method according to claim 13 wherein said sample comprises formalin fixed paraffin embedded (FFPE) tissue.

15. A method according to claim 13, further comprising:
(iiia) contacting said DNA with NEBNext FFPE Repair mix.

16. A method according to claim 13, further comprising:
(v) contacting said DNA with at least one dsDNA specific nuclease and at least one ssDNA specific nuclease and incubating to allow digestion.

17. A method according to claim 16 wherein said dsDNA specific nuclease comprises Lambda exo and said ssDNA specific nuclease comprises ExoI.

18. A method according to claim 13, further comprising:
(vi) purification of nucleic acid;
(vii) amplification of nucleic acid;
(viii) selecting nucleic acids in the range 300 to 450 bp;
(ix) determining the nucleotide sequence of one or more individual nucleic acid molecule(s);
(x) determining a mutational signature from the nucleotide sequence of step (ix); or
(xi) determining a homologous recombination deficiency signature from the nucleotide sequence of step (ix).

19-23. (canceled)

24. A kit comprising a pair of oligonucleotide adapters according to claim 1, a DNA ligase and at least two restriction enzymes, each restriction enzyme leaving a different sticky end upon nucleic acid cleavage, and optionally one or more of: buffer, one or more FFPE repair enzyme(s), one or more exonucleases.

25. (canceled)

26. A method for generation of a DNA library, comprising the step of ligation of one or more adapter(s) according to claim 1 to one or more double stranded DNA fragment(s) comprising a single stranded overhang at each end of said fragment(s).
